(19) United States
(12) Patent Application Publication
(10) Pub. No.: US 2023/0058774 A1
(43) Pub. Date: Feb. 23, 2023

(54) NOVEL DOMINANT NEGATIVE FAS POLYPEPTIDES, CELLS COMPRISING THEREOF AND USES THEREOF

(71) Applicants: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(72) Inventors: Christopher A. Klebanoff, New York, NY (US); Fei Yi, New York, NY (US)

(73) Assignees: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(21) Appl. No.: 17/858,320

(22) Filed: Jul. 6, 2022

Related U.S. Application Data
(63) Continuation of application No. PCT/US2021/012306, filed on Jan. 6, 2021.
(60) Provisional application No. 62/957,608, filed on Jan. 6, 2020.

(51) Int. Cl. C07K 14/705; C07K 14/725; A61P 37/04; A61P 35/00; A61K 35/17

(52) U.S. Cl. CPC C07K 14/70578; C07K 14/7051; A61P 37/04; A61P 35/00; A61K 35/17; C07K 2319/03; C07K 2319/02; C07K 2319/33; A61K 2039/5156

(57) ABSTRACT

The present disclosure provides novel dominant negative Fas polypeptides comprising a first modification in the cytoplasmic domain and a second modification in the N-terminal region of human Fas. The present disclosure also provides cells comprising such novel dominant negative Fas polypeptides and an antigen-recognizing receptor (e.g., a chimeric antigen receptor (CAR) or a T cell receptor (TCR)). Also provided are uses of the cells for treatment, e.g., for treating tumors and pathogen infections.

### CROSS-REFERENCE TO RELATED APPLICATIONS

The present application is a continuation application of International Patent Application No. PCT/US2021/012306, filed on Jan. 6, 2021, which claims priority to U.S. Provisional Patent Application No. 62/957,608, filed on Jan. 6, 2020, the contents of each of which are incorporated by reference in their entireties, and to each of which priority is claimed.

### SEQUENCE LISTING

The specification further incorporates by reference the Sequence Listing submitted herewith via EFS on Jul. 6, 2022. Pursuant to 37 C.F.R. § 1.52(e)(5), the Sequence Listing file, identified as 072734_1376.xml, is 109,677 bytes and was created on Jul. 6, 2022. The Sequence Listing, electronically filed herewith, does not extend beyond the scope of the specification and thus does not contain new matter.

### INTRODUCTION

The present disclosure provides novel dominant negative Fas polypeptides comprising a first modification in the cytoplasmic domain and a second modification in the N-terminal region of human Fas. The present disclosure also provides cells comprising such novel dominant negative Fas polypeptides and an antigen-recognizing receptor (e.g., a chimeric antigen receptor (CAR) or a T cell receptor (TCR)). Also provided are uses of the cells for treatment, e.g., for treating tumors and pathogen infections.

### BACKGROUND OF THE INVENTION

Adoptive cell immunotherapy with genetically engineered autologous or allogeneic T cells and NK cells has shown evidence of therapeutic efficacy in a range of human cancers, including but not limited to melanoma and various B-cell malignancies. T cells may be modified to target tumor-associated antigens through the introduction of genes encoding a receptor, e.g., a chimeric antigen receptor (CAR) or a T cell receptors (TCR), conveying specificity to antigens expressed by cancers or virally infected cells. Such engineered immune cells are a type of targeted immunotherapy that has the potential to provide for the treatment of cancer or infectious disease.

Adoptive cell transfer (ACT) using genetically engineered T cells has entered the standard of care for patients with refractory B cell malignancies, including pediatric acute lymphoblastic leukemia (1) and adult aggressive B cell lymphomas (2). The exceptional efficacy of ACT in hematologic lymphoid malignancies has been consistently observed across clinical trials, regardless of institution, gene vector, or cell composition (3-8). By contrast, responses to adoptive immunotherapy in patients with solid malignancies, collectively the leading cause of adult cancer-related deaths (9), have been comparatively modest (10-13). Thus, there is still a need for new strategies that enhance the potency of transferred T cells.

### SUMMARY OF THE INVENTION

The presently disclosed subject matter provides novel dominant negative Fas polypeptides comprising a first modification in the cytoplasmic domain and a second modification in the N-terminal region of human Fas. The presently disclosed subject matter also provides cells comprising such novel dominant negative Fas polypeptides and an antigen-recognizing receptor (e.g., a chimeric antigen receptor (CAR) or a T cell receptor (TCR)). Also provided are uses of the cells for treatment, e.g., for treating tumors and pathogen infections.

The presently disclosed subject matter provides dominant negative Fas polypeptides comprising a first modification in the cytoplasmic death domain and a second modification in the N-terminal region of human Fas. In certain embodiments, the first and second modifications each are independently selected from the group consisting of substitutions, deletions, and insertions. In certain embodiments, the substitution is a point mutation.

In certain embodiments, the first modification comprises or consists of a deletion of amino acids 230-314 of human Fas. In certain embodiments, the first modification consists of a deletion of amino acids 230-314 of human Fas.

In certain embodiments, the first modification comprises or consists of a point mutation at position 260 of human Fas. In certain embodiments, the first modification consists of a point mutation at position 260 of human Fas. In certain embodiments, the point mutation is D260V.

In certain embodiments, the second modification is located between the peptide signal region and cysteine rich domain 1 of human Fas. In certain embodiments, the peptide signal region is encoded by amino acids 1 to 25 of human Fas. In certain embodiments, the cysteine rich domain 1 of is encoded by amino acids 48 to 82 of human Fas.

In certain embodiments, the second modification comprises or consists of a modification at position 32 of human Fas. In certain embodiments, the second modification comprises or consists of a deletion of amino acid 32 of human Fas. In certain embodiments, the second modification consists of a deletion of amino acid 32 of human Fas.

In certain embodiments, the dominant negative Fas polypeptide comprises an amino acid sequence that is at least about 80%, at least about 85%, at least about 90%, at least about 95%, at least about 96%, at least about 97%, at least about 98%, at least about 99%, or at least about 100% identical to the amino acid sequence set forth in SEQ ID NO: 16. In certain embodiments, the dominant negative Fas polypeptide comprises or consists of the amino acid sequence set forth in SEQ ID NO: 16.

In certain embodiments, the second modification further comprises a second modification at position 31. In certain embodiments, the second modification comprises or consists of a deletion of amino acids 31 and 32 of human Fas. In certain embodiments, the first modification consists of a deletion of amino acids 230-314 of human Fas. In certain embodiments, the dominant negative Fas polypeptide comprises an amino acid sequence that is at least about 80%, at least about 85%, at least about 90%, at least about 95%, at least about 96%, at least about 97%, at least about 98%, at least about 99%, or at least about 100% identical to the amino acid sequence set forth in SEQ ID NO: 18. In certain embodiments, the dominant negative Fas polypeptide comprises or consists of the amino acid sequence set forth in SEQ ID NO: 18.

In certain embodiments, the second modification further comprises a second modification at position 33. In certain embodiments, the second modification comprises or consists of a deletion of the amino acids at positions 32 and 33 of human Fas. In certain embodiments, the first modification consists of a deletion of amino acids 230-314 of human Fas. In certain embodiments, the dominant negative Fas polypeptide comprises an amino acid sequence that is at least about 80%, at least about 85%, at least about 90%, at least about 95%, at least about 96%, at least about 97%, at least about 98%, at least about 99%, or at least about 100% identical to the amino acid sequence set forth in SEQ ID NO: 20. In certain embodiments, the dominant negative Fas polypeptide comprises or consists of the amino acid sequence set forth in SEQ ID NO: 20.

In certain embodiments, the second modification comprises or consists of a modification at position 33 of human Fas. In certain embodiments, the second modification further comprises a second modification at position 34. In certain embodiments, the second modification comprises or consists of a deletion of amino acids 33 and 34 of human Fas. In certain embodiments, the first modification consists of a deletion of amino acids 230-314 of human Fas. In certain embodiments, the dominant negative Fas polypeptide comprises an amino acid sequence that is at least about 80%, at least about 85%, at least about 90%, at least about 95%, at least about 96%, at least about 97%, at least about 98%, at least about 99%, or at least about 100% identical to the amino acid sequence set forth in SEQ ID NO: 22. In certain embodiments, the dominant negative Fas polypeptide comprises or consists of the amino acid sequence set forth in SEQ ID NO: 22.

In certain embodiments, the second modification comprises or consists of a point mutation at position 32 of human Fas. In certain embodiments, the second modification comprises or consists of a point mutation S32A of human Fas. In certain embodiments, the first modification consists of a deletion of amino acids 230-314 of human Fas. In certain embodiments, the dominant negative Fas polypeptide comprises an amino acid sequence that is at least about 80%, at least about 85%, at least about 90%, at least about 95%, at least about 96%, at least about 97%, at least about 98%, at least about 99%, or at least about 100% identical to the amino acid sequence set forth in SEQ ID NO: 24. In certain embodiments, the dominant negative Fas polypeptide comprises or consists of the amino acid sequence set forth in SEQ ID NO: 24.

In certain embodiments, the human Fas comprises or consists of the amino acid sequence set forth in SEQ ID NO: 10.

In certain embodiments, the first modification prevents the binding between the dominant negative Fas polypeptide and a FADD polypeptide. In certain embodiments, the second modification increases (a) the surface expression of the dominant negative Fas polypeptide by a cell, and/or the transduction efficiency of the dominant negative Fas polypeptide into a cell, and/or (c) the protection of the dominant negative Fas polypeptide from FasL-induced apoptosis.

The presently disclosed subject matter provides cells comprising a) an antigen-recognizing receptor that binds to an antigen, and b) a dominant negative Fas polypeptide disclosed herein. In certain embodiments, the dominant negative Fas polypeptide enhances cell persistence. In certain embodiments, the dominant negative Fas polypeptide reduces apoptosis or anergy of the cell. In certain embodiments, the antigen-recognizing receptor is exogenous or endogenous. In certain embodiments, the antigen-recognizing receptor is expressed from a vector. In certain embodiments, the dominant negative Fas polypeptide is expressed from a vector.

In certain embodiments, the cell is an immunoresponsive cell. In certain embodiments, the cell is a cell of the lymphoid lineage or a cell of the myeloid lineage. In certain embodiments, the cell is selected from the group consisting of a T cell, a Natural Killer (NK) cell, a B cell, a monocyte and a macrophage. In certain embodiments, the cell is a T cell. In certain embodiments, the T cell is a cytotoxic T lymphocyte (CTL), a regulatory T cell (T_{reg}), or a Natural Killer T (NKT) cell. In certain embodiments, the cell is a NK cell. In certain embodiments, cell is autologous or allogeneic to the intended recipient.

In certain embodiments, the antigen is a tumor antigen or a pathogen antigen. In certain embodiments, the antigen is a tumor antigen. In certain embodiments, the antigen is a tumor-specific antigen. In certain embodiments, the antigen is a tumor-associated antigen. In certain embodiments, the tumor antigen is selected from the group consisting of CD19, MUC16, MUC1, CAIX, CEA, CD8, CD7, CD10, CD20, CD22, CD30, CLL1, CD33, CD34, CD38, CD41, CD44, CD49f, CD56, CD74, CD133, CD138, EGP-2, EGP-40, EpCAM, Erb-B2, Erb-B3, Erb-B4, FBP, Fetal acetylcholine receptor, folate receptor-α, GD2, GD3, HER-2, hTERT, IL-13R-α2, κ-light chain, KDR, mutant KRAS, mutant HRAS, mutant PIK3CA, mutant IDH, mutant p53, mutant NRAS, LeY, L1 cell adhesion molecule, MAGE-A1, Mesothelin, MAGEA3, CT83 (also known as KK-LC-1), p53, MART1, GP100, Proteinase3 (PR1), Tyrosinase, Survivin, hTERT, EphA2, NKG2D ligands, NY-ESO-1, oncofetal antigen (h5T4), PSCA, PSMA, ROR1, TAG-72, VEGF-R2, WT-1, BCMA, CD123, CD44V6, NKCS1, EGF1R, EGFR-VIII, CD99, CD70, ADGRE2, CCR1, LILRB2, PRAME, HPV E6 oncoprotein, HPV E7 oncoprotein, and ERBB. In certain embodiments, the antigen is CD19.

In certain embodiments, the antigen is a pathogen-associated antigen. In certain embodiments, the pathogen-associated antigen is a viral antigen present in Cytomegalovirus (CMV), a viral antigen present in Epstein Barr Virus (EBV), a viral antigen present in Human Immunodeficiency Virus (HIV), or a viral antigen present in influenza virus.

In certain embodiments, the antigen-recognizing receptor is a T cell receptor (TCR) or a chimeric antigen receptor (CAR). In certain embodiments, the antigen-recognizing receptor is a TCR that recognizes a pathogen-associated antigen, and the cell is a pathogen-specific T cell. In certain embodiments, the antigen-recognizing receptor is a TCR that recognizes a tumor antigen, and the cell is a tumor-specific T cell. In certain embodiments, the TCR is an endogenous TCR or a recombinant TCR.

In certain embodiments, the antigen-recognizing receptor is a CAR. In certain embodiments, the CAR comprises an extracellular antigen-binding domain, a transmembrane domain, and an intracellular signaling domain. In certain embodiments, the intracellular signaling domain comprises a native CD3ζ polypeptide. In certain embodiments, the intracellular signaling domain comprises a modified CD3ζ polypeptide. In certain embodiments, the modified CD3ζ polypeptide comprises a native ITAM1, an ITAM2 variant consisting of two loss-of-function mutations, and an ITAM3 consisting of two loss-of-function mutations. In certain embodiments, the intracellular signaling domain further comprises at least one co-stimulatory signaling region. In certain embodiments, the at least one co-stimulatory signaling region comprises a CD28 polypeptide, a 4-1BB polypeptide, an OX40 polypeptide, an ICOS polypeptide, a DAP-10 polypeptide, or a combination thereof. In certain embodiments, the at least one co-stimulatory signaling region comprises a CD28 polypeptide.

In certain embodiments, the cell further comprises a suicide gene. In certain embodiments, the suicide gene is a Herpes simplex virus thymidine kinase (hsv-tk), inducible Caspase 9 Suicide gene (iCasp-9) or a truncated human epidermal growth factor receptor (EGFRt) polypeptide.

The presently disclosed subject matter further provides nucleic acid compositions comprising (a) a first nucleic acid sequence encoding an antigen-recognizing receptor that binds to an antigen, and (b) a second nucleic acid sequence encoding a dominant negative Fas polypeptide disclosed herein. In certain embodiments, one or both of the first and second nucleic acid sequences are operably linked to a promoter element. In certain embodiments, one or both of the first and second nucleic acid sequences are present on a vector. In certain embodiments, the vector is a retroviral vector. In certain embodiments, the vector is a lentiviral vector. The presently disclosed subject matter further provides cells comprising any of the nucleic acid compositions disclosed herein.

The presently disclosed subject matter further provides vectors comprising any of the nucleic acid compositions disclosed herein and cells comprising any of the vectors disclosed herein.

Furthermore, the presently disclosed subject matter provides pharmaceutical compositions comprising an effective amount of any of the cells disclosed herein and a pharmaceutically acceptable excipient.

In certain embodiments, the pharmaceutical composition is for treating and/or preventing a neoplasm or a pathogen infection.

Furthermore, the presently disclosed subject matter provides methods of inducing and/or enhancing an immune response to a target antigen. In certain embodiments, the method comprises administering to the subject an effective amount of any of the cells or pharmaceutical compositions disclosed herein.

The presently disclosed subject matter provides methods of reducing tumor burden in a subject. In certain embodiments, the method comprises administering to the subject an effective amount of the cells disclosed herein or a pharmaceutical composition disclosed herein. In certain embodiments, the method reduces the number of tumor cells, reduces tumor size, and/or eradicates the tumor in the subject.

Furthermore, the presently disclosed subject matter provides methods of treating and/or preventing a neoplasm. In certain embodiments, the method comprises administering to the subject an effective amount of any of the cells or pharmaceutical compositions disclosed herein.

Also provided are method of lengthening survival of a subject having a neoplasm. In certain embodiments, the method comprises administering to the subject an effective amount of the cells disclosed herein or a pharmaceutical composition disclosed herein. In certain embodiments, the neoplasm is a malignant neoplasm.

In certain embodiments, the tumor or neoplasm is selected from the group consisting of B cell leukemia, multiple myeloma, acute lymphoblastic leukemia (ALL), chronic lymphocytic leukemia (CLL), non-Hodgkin's lymphoma, myeloid leukemias, and myelodysplastic syndrome (MDS). In certain embodiments, the tumor or neoplasm is a solid tumor. In certain embodiments, the solid tumor is a tumor originating from the brain, breast, lung, gastro-intestinal tract (including esophagus, stomach, small intestine, large intestine, and rectum), pancreas, prostate, soft tissue/bone, uterus, cervix, ovary, kidney, skin, thymus, testis, head and neck, or liver.

Furthermore, the presently disclosed subject matter provides methods of preventing and/or treating a pathogen infection in a subject. In certain embodiments, the method comprises administering to the subject an effective amount of any of the cells or pharmaceutical compositions disclosed herein. In certain embodiments, the pathogen is selected from the group consisting of a virus, a bacterium, a fungus, a parasite and a protozoan capable of causing disease.

The presently disclosed subject matter also provides methods for producing an antigen-specific cell. In certain embodiments, the method comprises introducing into a cell (a) a first nucleic acid sequence encoding an antigen-recognizing receptor that binds to an antigen; and (b) a second nucleic sequence encoding a dominant negative Fas polypeptide disclosed herein. In certain embodiments, one or both of the first and second nucleic acid sequences are operably linked to a promoter element. In certain embodiments, one or both of the first and second nucleic acid sequences are present on a vector. In certain embodiments, the vector is a retroviral vector.

The presently disclosed subject matter also provides kits comprising a cell disclosed herein, a nucleic acid composition disclosed herein, or a vector disclosed herein. In certain embodiments, the kit further comprises written instructions for treating and/or preventing a neoplasm or a pathogen infection.

### BRIEF DESCRIPTION OF THE FIGURES

The following Detailed Description, given by way of example, but not intended to limit the presently disclosed subject matter to specific embodiments described, may be understood in conjunction with the accompanying drawings.

FIGS. 1A-1E show generation of cells comprising a chimeric antigen receptor (CAR) and a dominant negative Fas polypeptide and their activities. FIG. 1A shows two versions of a human FasDNR construct design. EGFRt represents truncated EGFR. P2A represents porcine teschovirus self-cleavage peptide sequence. FasDNR represents Fas dominant negative receptor. Ψ represents retroviral packaging signal. 192ζ1XXCAR represents an anti-CD19 chimeric antigen receptor comprising an intracellular domain that comprises a modified CD3ζ and a co-stimulatory signaling region that comprises a CD28 polypeptide. FIG. 1B is schematic representation of edited T cells. 192ζ1XXCAR targeted CD19⁺ malignant cells. FasDNR protected T cells from FasL induced apoptosis. When administered with Cetuximab, EGFRt can be targeted and induced antibody-dependent cell-mediated cytotoxicity (ADCC) or complement-dependent cytotoxicity.

FIG. 1C shows that human-derived Jurkat cells retrovirally transduced with EGFRt alone or EGFRt/FasDNR. Cells were stained on day 2 post-transduction. FIG. 1D shows intracellular staining of TNFα on primary human CD8⁺ T cells transduced with a TCR targeting NY-ESO antigen. Cells with or without FasDNR were exposed to antigen for 6 hour before intracellular cytokine staining. FIG. 1E shows primary human CD8⁺ T cells exposed to 100 ng/ml of FasL leucine zipper (FasL-1z) at designated time points. Activated Caspase3/7 and Annexin V were used as early apoptosis markers.

FIGS. 2A-2C depict expression of Fas of single clones selected from CRISPR edited Jurkat cells. FIG. 2A shows Jurkat cells electroporated with recombinant Cas9 protein loaded with synthetic guide (sg)RNA targeting the human Fas gene in exon 2. Fas surface expressions were measured on edited T cells following single cell cloning approx. 3 weeks after electroporation. WT represents wildtype gene sequence for Fas. FIG. 2B shows data that are presented as the comparison of WI of Fas expression levels, p value was determined by paired Student's t-tests comparing clone #15 and #17 (*** p<0.001). FIG. 2C shows the summary of Fas gene sequences from each Jurkat clone. Underlines indicate guide RNA target sequence. Sequences in bold indicate deletions. FIG. 2C discloses SEQ ID NOS: 69-74, respectively, in order of appearance.

FIGS. 3A and 3B depict response of clone 17 Jurkat cells to FasL stimulation. FIG. 3A shows apoptosis assay of single clone Jurkat cells post CRISPR editing. Cells were treated with 100 ng/ml FasL-LZ at designated time points. Clone #15, wild type Fas exon 2 sequence. Clone #17, mono-allelic 6 bp (2 codon) in-frame deletion. Clone #19, bi-allelic 19 bp (frameshift) deletion. FIG. 3B shows triplicated data of the same apoptosis assay. Vertical bars indicate comparison between clone #15 and #17 at each time point, p value was determined by paired Student's t-tests of each matched sample (*** p<0.001, **** p<0.0001).

FIGS. 4A and 4B depict protection of Fas knock-out clone 19 and FasDNR+ Jurkat cells from FasL induced apoptosis. FIG. 4A shows Jurkat cells exposed to 100 ng/ml of FasL-LZ at designated time points. Clone #15 WT, wild type Fas exon 2 sequence. Clone #19 indel-19, Fas exon 2 with 19 bp deletion calculated by ICE sequencing software. EGFRt⁺ and EGFRt⁺ FasDNR⁺ represent Jurkat cells gated on EGFRt positive (control group) or EGFRt and FasDNR double positive cells respectively. FIG. 4B shows triplicated data of the same apoptosis assay represented by early apoptosis markers described before.

FIG. 5 shows prediction of expression enhancing mutants in human Fas N-terminal region. A summarized map of Fas protein (335 amino acid) functional regions. PLAD, pre-ligand assembly domains. CRD, cysteine-rich domains. TM, transmembrane domain. DD, death domain. DNR del 222-306 indicates the FasDNR truncated region. S32 (serine at amino acid 32) is the predicted mutation site that contributed to clone #17's phenotype. Del 32, deletion of S32. Del31-32, deletion of N31 and S32. Del32-33, deletion of S32 and K33. S32A, substitution of amino acid 32 from S to A.

FIGS. 6A and 6B depict enhanced Fas transduction efficiency of Fas N-terminal mutants. Clone 19 Fas knock-out Jurkat cells transduced with Fas or FasDNR with S32 mutations retroviral vectors. Cells were stained for Fas expression on day 3 post viral transduction. Solid gray, control group cells transduced with Fas WT (FIG. 6A) or FasDNR (FIG. 6B). Blank dotted, cells transduced with Fas S32 mutants.

FIGS. 7A and 7B depict results for an apoptosis assay in Jurkat cells. Induction of apoptosis was conducted using a recombinant Fas ligand (CD178) oligomerized through a leucine zipper domain (lz-FASL) to mimic the naturally occurring active form of the ligand. FASL is known to trigger apoptosis by binding with FAS receptor on a responding cell. Cells were transduced with FASDNR with or without S32 mutations and incubated with recombinant Fas ligand. FIG. 7A shows FACS analysis of active Caspase 3 and Caspase 7 (x-axis) and of Annexin-V staining (Annex V bound to plasma membrane of cells undergoing apoptosis; y-axis) at different time-points. Caspase 3 and Caspase 7 are downstream signaling molecules that are involved in FAS signaling. Activation of Caspase 3 and 7 are required for apoptosis initiation. FIG. 7B shows the quantitative analysis of the apoptosis measurements during time.

FIG. 8 depicts the Fas expression in human natural killer cells (NK cells) upon activation by human IL-2 and irradiated K562 clone9 cells.

FIG. 9 depicts the expression of Fas in human NK cells transduced with EGFRt/1928z, EGFRt/1928z/FasDNR or EGFRt/1928z/FasDNR del31-32.

FIG. 10 depicts that expressing N-terminal mutant Fas DNR in NK cells resulted in significantly increased cell numbers as compared to Fas DNR modified and unmodified NK cells following the exposure to Fas ligand.

### DETAILED DESCRIPTION OF THE INVENTION

The presently disclosed subject matter provides novel dominant negative Fas polypeptides and cells comprising such polypeptides. In certain embodiments, the cell further comprises an antigen-recognizing receptor (e.g., a TCR or a CAR). The presently disclosed subject matter also provides methods of using such cells for treating and/or preventing neoplasms and pathogen infections. The presently disclosed subject matter is based, at least in part, on the discovery that the presence of a modification in the N-terminal region of a dominant negative Fas polypeptide increases the surface expression of the dominant negative Fas polypeptide by a cell, and/or increases the transduction efficiency of the dominant negative Fas polypeptide into a cell, and/or increases the protection of the dominant negative Fas polypeptide from FasL-induced apoptosis.

### 1. Definitions

Unless defined herein, all technical and scientific terms used in this detailed description have the meaning commonly understood by a person skilled in the art of immune oncology as reflected, for example, in general definitions of many of the terms used in the presently disclosed subject matter included in one or more of the following: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991).

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold or within 2-fold, of a value.

By “immunoresponsive cell” is meant a cell that functions in an immune response or a progenitor, or progeny thereof, including cells that initiate, activate, and/or regulate (increase or decrease) an immune response.

By “activates an immunoresponsive cell” is meant induction of signal transduction or changes in protein expression in the cell resulting in initiation of an immune response. For example, when CD3 chains cluster in response to ligand binding and immunoreceptor tyrosine-based inhibition motifs (ITAMs), a signal transduction cascade is produced. In certain embodiments, binding of a TCR or a CAR to an antigen leads to a formation of an immunological synapse that includes clustering of many molecules near the bound receptor (e.g. CD4 or CD8, CD3γ/δ/ε/ζ, etc.). This clustering of membrane bound signaling molecules allows ITAM motifs contained within the CD3 chains to become phosphorylated. This phosphorylation in turn initiates a T cell activation pathway ultimately activating transcription factors, such as NF-κB and AP-1. These transcription factors induce global gene expression of the T cell to increase IL-2 production for proliferation and expression of master regulator T cell proteins in order to initiate a T cell mediated immune response.

By “stimulates an immunoresponsive cell” is meant a signal that results in a robust and sustained immune response. In various embodiments, this occurs after immune cell (e.g., T-cell) activation or is concomitantly mediated through receptors including, but not limited to, CD28, CD137 (4-1BB), OX40, CD40 and ICOS. Receiving multiple stimulatory signals can be important to mount a robust and long-term T-cell mediated immune response, but T cells receiving multiple stimulatory signals can quickly become inhibited and unresponsive to antigen, a state commonly referred to as “exhaustion”. While the effects of these co-stimulatory signals may vary, they generally result in increased gene expression in order to generate long lived, proliferative, and anti-apoptotic T cells that robustly respond to antigen for complete and sustained eradication.

The term “antigen-recognizing receptor” as used herein refers to a receptor that is capable of activating an immunoresponsive cell (e.g., a T-cell) in response to its binding to an antigen. Non-limiting examples of antigen-recognizing receptors include native or endogenous T cell receptors (“TCRs”), and chimeric antigen receptors (“CARs”).

As used herein, the term “antibody” means not only intact antibody molecules, but also fragments of antibody molecules that retain antigen-binding ability. Such fragments are also well known in the art and are regularly employed both in vitro and in vivo. Accordingly, as used herein, the term “antibody” means not only intact immunoglobulin molecules but also the well-known active fragments F(ab)₂, and Fab. F(ab′)₂, and Fab fragments that lack the Fe fragment of intact antibody, clear more rapidly from the circulation, and may have less non-specific tissue binding than an intact antibody (Wahl et al., J. Nucl. Med. 24:316-325 (1983). As used herein, the term “antibodies” encompasses whole native antibodies, bispecific antibodies; chimeric antibodies; Fab, Fab′, single chain V region fragments (scFv), and fusion polypeptides.

As used herein, the term “complementarity determining region(s)” or “CDRs” refers to the hypervariable regions of the immunoglobulin heavy and light chain amino acid sequences. See, e.g., Kabat et al., Sequences of Proteins of Immunological Interest, 4th U. S. Department of Health and Human Services, National Institutes of Health (1987). Generally, antibodies comprise three heavy chain and three light chain CDRs or CDR regions in the variable region. CDRs provide the majority of contact residues for the binding of the antibody to its cognate antigen or epitope. In certain embodiments, the CDRs regions are numbered using the Kabat system (Kabat, E. A., et al. (1991) Sequences of Proteins of Immunological Interest, Fifth Edition, U.S. Department of Health and Human Services, NIH Publication No. 91-3242).

As used herein, the term “single-chain variable fragment” or “scFv” is a fusion protein of the variable regions of the heavy (V_{H}) and light chains (V_{L}) of an immunoglobulin covalently linked to form a V_{H}::V_{L} heterodimer. The V_{H} and V_{L} are either joined directly or joined by a peptide-encoding linker (e.g., 10, 15, 20, 25 amino acids), which connects the N-terminus of the V_{H} with the C-terminus of the V_{L}, or the C-terminus of the V_{H} with the N-terminus of the V_{L}. The linker is usually rich i glycine for flexibility, as well as serine or threonine for solubility. Despite removal of the constant regions and the introduction of a linker, scFv proteins retain the specificity of the original immunoglobulin. Single chain Fv polypeptide antibodies can be expressed from a nucleic acid including V_{H}- and V_{L}-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008 27(6):455-51; Peter et al., J Cachexia Sarcopenia Muscle 2012 Aug. 12; Shieh et al., J Imunol 2009 183(4):2277-85; Giomarelli et al., Thromb Haemost 2007 97(6):955-63; Fife eta., J Clin Invst 2006 116(8):2252-61; Brocks et al., Immunotechnology 1997 3(3):173-84; Moosmayer et al., The Immunol 1995 2(10:31-40). Agonistic scFvs having stimulatory activity have been described (see, e.g., Peter et al., J Biol Chem 2003 25278(38):36740-7; Xie et al., Nat Biotech 1997 15(8):768-71; Ledbetter et al., Crit Rev Immunol 1997 17(5-6):427-55; Ho et al., Bio Chim Biophys Acta 2003 1638(3):257-66).

As used herein, the term “affinity” is meant a measure of binding strength. Affinity can depend on the closeness of stereochemical fit between antibody combining sites and antigen determinants, on the size of the area of contact between them, and/or on the distribution of charged and hydrophobic groups. Methods for calculating the affinity of an antibody for an antigen are known in the art, including, but not limited to, various antigen-binding experiments, e.g., functional assays (e.g., flow cytometry assay).

The term “chimeric antigen receptor” or “CAR” as used herein refers to a molecule (e.g., a synthetic receptor) comprising an extracellular antigen-binding domain fused to an intracellular signaling domain that is capable of activating or stimulating an immunoresponsive cell. In certain embodiments, the CAR further comprises a transmembrane domain. In certain embodiments, the extracellular antigen-binding domain of a CAR comprises an scFv. The scFv can be derived from fusing the variable heavy and light regions of an antibody. In certain embodiments, the scFv may be derived from Fab's (instead of from an antibody, e.g., obtained from Fab libraries). In certain embodiments, the scFv is fused to the transmembrane domain and then to the intracellular signaling domain. In certain embodiments, the CAR is selected to have high binding affinity or avidity for the antigen.

As used herein, the term “nucleic acid molecules” includes any nucleic acid molecule that encodes a polypeptide of interest (e.g., a dominant negative Fas polypeptide or an antigen-recognizing receptor) or a fragment thereof. Such nucleic acid molecules need not be 100% homologous or identical with an endogenous nucleic acid sequence, but may exhibit substantial identity. Polynucleotides having “substantial identity” or “substantial homology” to an endogenous sequence are typically capable of hybridizing with at least one strand of a double-stranded nucleic acid molecule.

As used herein, the term “a conservative sequence modification” refers to an amino acid modification that in a protein in which an amino acid having a particular physicochemical characteristic is substituted for another amino acid having the same physicochemical characteristic (e.g., substituting one basic amino acid for another). In certain embodiments, such substitutions are less likely to have a significant impact on the activity of the protein (e.g., a conservative substitution in an antibody CDR would be less likely to significantly affect or alter the binding characteristics of the protein). Modifications can be introduced into the human scFv of the presently disclosed CAR by standard techniques known in the art, such as site-directed mutagenesis and PCR-mediated mutagenesis. Amino acids can be classified into groups according to their physicochemical properties such as charge and polarity. In certain embodiments, the conservative modification is a conservative amino acid substitution. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. In certain embodiments, conservative substitutions include substitutions within the following groups: glycine, alanine; valine, isoleucine, leucine; aspartic acid, glutamic acid, asparagine, glutamine; serine, threonine; lysine, arginine; and phenylalanine, tyrosine. In certain embodiments, one or more amino acid residues within or outside a CDR region can be replaced with other amino acid residues from the same group and the altered antibody can be tested for retained function (i.e., the functions set forth in (c) through (l) above) using the functional assays described herein. In certain embodiments, no more than one, no more than two, no more than three, no more than four, no more than five residues within a specified sequence outside a CDR region or a CDR region are altered.

In certain embodiments, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm.

The percent homology or identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

In certain embodiments, the amino acids sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

Furthermore, sequence identity can be measured by using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications.

By “substantially identical” or “substantially homologous” is meant a polypeptide or nucleic acid molecule exhibiting at least about 50% homologous or identical to a reference amino acid sequence (for example, any one of the amino acid sequences described herein) or nucleic acid sequence (for example, any one of the nucleic acid sequences described herein). In certain embodiments, such a sequence is at least about 60%, at least about 65%, at least about 70%, at least about 75%, at least about 80%, at least about 85%, at least about 90%, at least about 95%, at least about 99%, or at least about 100% homologous or identical to the sequence of the amino acid or nucleic acid used for comparison.

In an exemplary approach to determining the degree of identity, a BLAST program may be used, with a probability score between e-3 and e-100 indicating a closely related sequence.

By “analog” is meant a structurally related polypeptide or nucleic acid molecule having the function of a reference polypeptide or nucleic acid molecule.

The term “ligand” as used herein refers to a molecule that binds to a receptor. In certain embodiments, the ligand binds to a receptor on another cell, allowing for cell-to-cell recognition and/or interaction.

The term “constitutive expression” or “constitutively expressed” as used herein refers to expression or expressed under all physiological conditions.

By “disease” is meant any condition, disease or disorder that damages or interferes with the normal function of a cell, tissue, or organ, e.g., neoplasm, and pathogen infection of cell.

An “effective amount” (or, “therapeutically effective amount”) is an amount sufficient to affect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the immunoresponsive cells administered.

By “enforcing tolerance” is meant preventing the activity of self-reactive cells or immunoresponsive cells that target transplanted organs or tissues.

By “endogenous” is meant a nucleic acid molecule or polypeptide that is normally expressed in a cell or tissue.

By “exogenous” is meant a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

By a “heterologous nucleic acid molecule or polypeptide” is meant a nucleic acid molecule (e.g., a cDNA, DNA or RNA molecule) or polypeptide that is not normally present in a cell or sample obtained from a cell. This nucleic acid may be from another organism, or it may be, for example, an mRNA molecule that is not normally expressed in a cell or sample.

By “modulate” is meant positively or negatively alter. Exemplary modulations include a about 1%, about 2%, about 5%, about 10%, about 25%, about 50%, about 75%, or about 100% change.

By “increase” is meant to alter positively by at least about 5%. An alteration may be by about 5%, about 10%, about 25%, about 30%, about 50%, about 75%, about 100% or more.

By “reduce” is meant to alter negatively by at least about 5%. An alteration may be by about 5%, about 10%, about 25%, about 30%, about 50%, about 75%, or even by about 100%.

By “isolated cell” is meant a cell that is separated from the molecular and/or cellular components that naturally accompany the cell.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

The term “antigen-binding domain” as used herein refers to a domain capable of specifically binding a particular antigenic determinant or set of antigenic determinants present on a cell.

“Linker”, as used herein, shall mean a functional group (e.g., chemical or polypeptide) that covalently attaches two or more polypeptides or nucleic acids so that they are connected to one another. As used herein, a “peptide linker” refers to one or more amino acids used to couple two proteins together (e.g., to couple V_{H} and V_{L} domains). In certain embodiments, the linker comprises a sequence set forth in GGGGSGGGGSGGGGS [SEQ ID NO: 1].

By “neoplasm” is meant a disease characterized by the pathological proliferation of a cell or tissue and its subsequent migration to or invasion of other tissues or organs. Neoplastic growth is typically uncontrolled and progressive, and occurs under conditions that would not elicit, or would cause cessation of, multiplication of normal cells. Neoplasm can affect a variety of cell types, tissues, or organs, including but not limited to an organ selected from the group consisting of bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasms include cancers, such as sarcomas, carcinomas, or plasmacytomas (malignant tumor of the plasma cells).

By “receptor” is meant a polypeptide, or portion thereof, present on a cell membrane that selectively binds one or more ligands.

By “recognize” is meant selectively binds to a target. A T cell that recognizes a tumor can expresses a receptor (e.g., a TCR or CAR) that binds to a tumor antigen.

By “reference” or “control” is meant a standard of comparison. For example, the level of scFv-antigen binding by a cell expressing a CAR and an scFv may be compared to the level of scFv-antigen binding in a corresponding cell expressing CAR alone.

By “secreted” is meant a polypeptide that is released from a cell via the secretory pathway through the endoplasmic reticulum, Golgi apparatus, and as a vesicle that transiently fuses at the cell plasma membrane, releasing the proteins outside of the cell.

By “signal sequence” or “leader sequence” is meant a peptide sequence (e.g., 5, 10, 15, 20, 25 or 30 amino acids) present at the N-terminus of newly synthesized proteins that directs their entry to the secretory pathway. Exemplary leader sequences include, but is not limited to, the IL-2 signal sequence: MYRMQLLSCIALSLALVTNS [SEQ ID NO: 2] (human), MYSMQLASCVTLTLVLLVNS [SEQ ID NO: 3] (mouse); the kappa leader sequence: METPAQLLFLLLLWLPDTTG [SEQ ID NO: 4] (human), METDTLLLWVLLLWVPGSTG [SEQ ID NO: 5] (mouse); the CD8 leader sequence: MALPVTALLLPLALLLHAARP [SEQ ID NO: 6] (human); the truncated human CD8 signal peptide: MALPVTALLLPLALLLHA [SEQ ID NO: 7] (human); the albumin signal sequence: MKWVTFISLLFSSAYS [SEQ ID NO: 8] (human); and the prolactin signal sequence: MDSKGSSQKGSRLLLLLVVSNLLLCQGVVS [SEQ ID NO: 9] (human). By “soluble” is meant a polypeptide that is freely diffusible in an aqueous environment (e.g., not membrane bound).

By “specifically binds” is meant a polypeptide or fragment thereof that recognizes and binds to a biological molecule of interest (e.g., a polypeptide), but which does not substantially recognize and bind other molecules in a sample, for example, a biological sample, which naturally includes a presently disclosed polypeptide.

The term “tumor antigen” as used herein refers to an antigenic substance produced in tumor cells. Tumor antigens can trigger an immune response in the host. As used herein, the term “tumor antigen” includes tumor-specific antigens (TSAs) and tumor-associated antigens (TAAs). TSAs refer to antigens that are uniquely or differentially expressed on a tumor cell as compared to a normal cell, e.g., present only on tumor cells and not on normal cells. In certain embodiments, a tumor antigen includes any polypeptide expressed by a tumor that is capable of activating or inducing an immune response via an antigen-recognizing receptor (e.g., CD19, MUC-16) or capable of suppressing an immune response via receptor-ligand binding (e.g., CD47, PD-L1/L2, B7.1/2). TAAs are antigens that are present on some tumor cells and also some normal cells.

The terms “comprises”, “comprising”, and are intended to have the broad meaning ascribed to them in U.S. Patent Law and can mean “includes”, “including” and the like.

As used herein, “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys. The term “immunocompromised” as used herein refers to a subject who has an immunodeficiency. The subject is very vulnerable to opportunistic infections, infections caused by organisms that usually do not cause disease in a person with a healthy immune system, but can affect people with a poorly functioning or suppressed immune system.

Other aspects of the presently disclosed subject matter are described in the following disclosure and are within the ambit of the presently disclosed subject matter.

### 2. Dominant Negative Fas Polypeptide

Fas cell surface death receptor (Fas) is also known as APT1; CD95; FAS1; APO-1; FASTM; ALPS1A; TNFRSF6. GenBank ID: 355 (human), 14102 (mouse), 246097 (rat), 282488 (cattle), 486469 (dog). The protein product of Fas includes, but is not limited to, NCBI Reference Sequences NP_000034.1, NP_001307548.1, NP_690610.1 and NP_690611.1.

Fas is a member of the TNF-receptor superfamily and contains a death domain. In human Fas, the death domain is encoded by amino acids 226-319. Fas is involved in the regulation of programmed cell death, and has been implicated in the pathogenesis of various malignancies and diseases of the immune system. The interaction of Fas with its ligand allows the formation of a cell death-inducing signaling complex with other components, e.g., Fas-associated protein with death domain (FADD), which can induce programmed cell death, also known as apoptosis.

In certain embodiments, the term “dominant negative Fas polypeptide” refers to the dominant negative form of a Fas polypeptide, which is a gene product of a dominant negative mutation of a Fas gene. In certain embodiments, a Fas polypeptide comprising a dominant negative mutation (also called “antimorphic mutations”) is an altered gene product that acts antagonistically to the wild-type Fas polypeptide. In certain embodiments, a dominant negative Fas polypeptide adversely affects the normal, wild-type Fas polypeptide within the same cell. In certain embodiments, the dominant negative Fas polypeptide interacts with a wild-type Fas polypeptide, but blocks its signal transduction to downstream molecules, e.g., FADD.

In certain embodiments, the dominant negative Fas polypeptide comprises a first modification in the intracellular domain and a second modification in the N-terminal region of human Fas. In certain embodiments, the first modification is within the cytoplasmic death domain. In certain embodiments, the first modification prevents the binding of Fas to a FADD polypeptide. In certain embodiments, the second modification is located between the peptide signal region and the cysteine rich domain 1 of Fas (e.g., human Fas). In certain embodiments, the peptide signal region of human Fas is encoded by amino acids 1 to 25 of human Fas. In certain embodiments, the cysteine rich domain 1 of human Fas is encoded by amino acids 48 to 82 of human Fas.

There were no known functions described for the region between the peptide signal region and the cysteine rich domain 1 of Fas (e.g., human Fas). Using a CRISPR/Cas9 screen, the inventors of this application discovered that the modification in this region (e.g., truncation of serine 32) enhances Fas cell surface expression. This function is independent from the first modification and can be combined with dominant negative Fas polypeptides to further improve their surface expressions and dominant negative functions.

In certain embodiments, the human Fas comprises or consists of the amino acid sequence of NCBI Reference No.: NP_000034.1 (SEQ ID NO: 10), which is provided below. In certain embodiments, human Fas polypeptide comprises or consists of an amino acid sequence that is at least about 80%, at least about 85%, at least about 90%, at least about 95%, at least about 96%, at least about 97%, at least about 98%, at least about 99%, or at least about 100% homologous or identical to the amino acid sequence set forth in SEQ ID NO: 10.

| (SEQ ID NO: 10) | |
|---|---|
| 1 | MLGIWTLLPL VLTSVARLSS KSVNAQVTDI NSKGLELRKT VTTVETQNLE GLHHDGQFCH |
| 61 | KPCPPGERKA RDCTVNGDEP DCVPCQEGKE YTDKAHFSSK CRRCRLCDEG HGLEVEINCT |
| 121 | RTQNTKCRCK PNFFCNSTVC EHCDPCTKCE HGIIKECTLT SNTKCKEEGS RSNLGWLCLL |
| 181 | LLPIPLIVWV KRKEVQKTCR KHRKENQGSH ESPTLNPETV AINLSDVDLS KYITTIAGVM |
| 241 | TLSQVKGFVR KNGVNEAKID EIKNDNVQDT AEQKVQLLRN WHQLHGKKEA YDTLIKDLKK |
| 301 | ANLCTLAEKI QTIILKDITS DSENSNFRNE IQSLV |

An exemplary nucleotide sequence encoding the amino acid sequence of SEQ ID NO: 10 is set forth in SEQ ID NO: 11, which is provided below.

(SEQ ID NO: 11)
ATGCTGGGCATCTGGACCCTCCTACCTCTGGTTCTTACGTCTGTTGCTAG
ATTATCGTCCAAAAGTGTTAATGCCCAAGTGACTGACATCAACTCCAAGG
GATTGGAATTGAGGAAGACTGTTACTACAGTTGAGACTCAGAACTTGGAA
GGCCTGCATCATGATGGCCAATTCTGCCATAAGCCCTGTCCTCCAGGTGA
AAGGAAAGCTAGGGACTGCACAGTCAATGGGGATGAACCAGACTGCGTGC
CCTGCCAAGAAGGGAAGGAGTACACAGACAAAGCCCATTTTTCTTCCAAA
TGCAGAAGATGTAGATTGTGTGATGAAGGACATGGCTTAGAAGTGGAAAT
AAACTGCACCCGGACCCAGAATACCAAGTGCAGATGTAAACCAAACTTTT
TTTGTAACTCTACTGTATGTGAACACTGTGACCCTTGCACCAAATGTGAA
CATGGAATCATCAAGGAATGCACACTCACCAGCAACACCAAGTGCAAAGA
GGAAGGATCCAGATCTAACTTGGGGTGGCTTTGTCTTCTTCTTTTGCCAA
TTCCACTAATTGTTTGGGTGAAGAGAAAGGAAGTACAGAAAACATGCAGA
AAGCACAGAAAGGAAAACCAAGGTTCTCATGAATCTCCAACCTTAAATCC
TGAAACAGTGGCAATAAATTTATCTGATGTTGACTTGAGTAAATATATCA
CCACTATTGCTGGAGTCATGACACTAAGTCAAGTTAAAGGCTTTGTTCGA
AAGAATGGTGTCAATGAAGCCAAAATAGATGAGATCAAGAATGACAATGT
CCAAGACACAGCAGAACAGAAAGTTCAACTGCTTCGTAATTGGCATCAAC
TTCATGGAAAGAAAGAAGCGTATGACACATTGATTAAAGATCTCAAAAAA
GCCAATCTTTGTACTCTTGCAGAGAAAATTCAGACTATCATCCTCAAGGA
CATTACTAGTGACTCAGAAAATTCAAACTTCAGAAATGAAATCCAAAGCT
TGGTC

2.1. First Modification

In certain embodiments, the first modification is within the cytoplasmic death domain of Fas. In certain embodiments, the first modification is within amino acids about 200 to about 320 of human Fas (e.g., one comprising or consisting of the amino acid sequence set forth in SEQ ID NO: 10). In certain embodiments, the first modification is within amino acids about 200 to about 319 of human Fas (e.g., one comprising or consisting of the amino acid sequence set forth in SEQ ID NO: 10). In certain embodiments, the first modification is within amino acids about 202 to about 319 of human Fas (e.g., one comprising or consisting of the amino acid sequence set forth in SEQ ID NO: 10). In certain embodiments, the first modification is within amino acids about 226 to about 319 of human Fas (e.g., one comprising or consisting of the amino acid sequence set forth in SEQ ID NO: 10). Death domains of Fas protein are disclosed in Tartaglia L A et al. Cell. (1993); 74(5):845-53; Itoh and Nagata. J Biol Chem. (1993); 268(15):10932; Boldin M P et al. J Biol Chem. (1995); 270(14):7795-8; and Huang B et al. Nature (1996); 384(6610):638-41, all of which are incorporated by reference herein.

In certain embodiments, the first modification is selected from the group consisting of substitutions, deletions, and insertions. In certain embodiments, the substitution is a point mutation.

In certain embodiments, the first modification is a deletion. In certain embodiments, the first modification comprises a partial or complete deletion of the death domain. In certain embodiments, the first modification comprises or consists of a deletion of amino acid residues 230-314 of a human wild-type Fas polypeptide (e.g., one comprising or consisting of the amino acid sequence set forth in SEQ ID NO: 10). In certain embodiments, the first modification consists of a deletion of amino acid residues 230-314 of a human wild-type Fas polypeptide (e.g., one comprising or consisting of the amino acid sequence set forth in SEQ ID NO: 10). In certain embodiments, the dominant negative polypeptide consists of a first modification that consists of a deletion of amino acid residues 230-314 of a human wild-type Fas consisting of the amino acid sequence set forth in SEQ ID NO: 10. In certain embodiments, the dominant negative polypeptide is designated as “hFas^{ΔDD}.” In certain embodiments, hFas^{ΔDD} comprises or consists of the amino acid sequence set forth in SEQ ID NO: 12. SEQ ID NO: 12 is provided below.

(SEQ ID NO: 12)
MLGIWTLLPLVLTSVARLSSKSVNAQVTDINSKGLELRKTVTTVETQNLE
GLHHDGQFCHKPCPPGERKARDCTVNGDEPDCVPCQEGKEYTDKAHFSSK
CRRCRLCDEGHGLEVEINCTRTQNTKCRCKPNFFCNSTVCEHCDPCTKCE
HGIIKECTLTSNTKCKEEGSRSNLGWLCLLLLPIPLIVWVKRKEVQKTCR
KHRKENQGSHESPTLNPETVAINLSDVDLLKDITSDSENSNFRNEIQSLV

An exemplary nucleotide sequence encoding the amino acid sequence of SEQ ID NO: 12 is set forth in SEQ ID NO: 13, which is provided below.

(SEQ ID NO: 13)
ATGCTGGGCATCTGGACCCTCCTACCTCTGGTTCTTACGTCTGTTGCTAG
ATTATCGTCCAAAAGTGTTAATGCCCAAGTGACTGACATCAACTCCAAGG
GATTGGAATTGAGGAAGACTGTTACTACAGTTGAGACTCAGAACTTGGAA
GGCCTGCATCATGATGGCCAATTCTGCCATAAGCCCTGTCCTCCAGGTGA
AAGGAAAGCTAGGGACTGCACAGTCAATGGGGATGAACCAGACTGCGTGC
CCTGCCAAGAAGGGAAGGAGTACACAGACAAAGCCCATTTTTCTTCCAAA
TGCAGAAGATGTAGATTGTGTGATGAAGGACATGGCTTAGAAGTGGAAAT
AAACTGCACCCGGACCCAGAATACCAAGTGCAGATGTAAACCAAACTTTT
TTTGTAACTCTACTGTATGTGAACACTGTGACCCTTGCACCAAATGTGAA
CATGGAATCATCAAGGAATGCACACTCACCAGCAACACCAAGTGCAAAGA
GGAAGGTTCCAGATCTAACTTGGGGTGGCTTTGTCTTCTTCTTTTGCCAA
TTCCACTAATTGTTTGGGTGAAGAGAAAGGAAGTACAGAAAACATGCAGA
AAGCACAGAAAGGAAAACCAAGGTTCTCATGAATCTCCAACCTTAAATCC
TGAAACAGTGGCAATAAATTTATCTGATGTTGACTTGCTCAAGGACATTA
CTAGTGACTCAGAAAATTCAAACTTCAGAAATGAAATCCAAAGCTTGGTC

In certain embodiments, the first modification consists of a point mutation. In certain embodiments, the first modification comprises or consists of a point mutation at position 260 of a human Fas polypeptide (e.g., one comprising or consisting of the amino acid sequence set forth in SEQ ID NO: 10). In certain embodiments, the point mutation consists of D260V. In certain embodiments, the first modification consists of a point mutation D260V of a human wild-type Fas polypeptide. In certain embodiments, the dominant negative polypeptide consists of a first modification that consists of a point mutation D260V of a human wild-type Fas polypeptide consisting of the amino acid sequence set forth in SEQ ID NO: 10. In certain embodiments, the dominant negative polypeptide is designated as “hFas^{D260V}”. In certain embodiments, hFas^{D260V} comprises or consists of the amino acid sequence set forth in SEQ ID NO: 14. SEQ ID NO: 14 is provided below.

(SEQ ID NO: 14)
MLGIWTLLPLVLTSVARLSSKSVNAQVTDINSKGLELRKTVTTVETQNLE
GLHHDGQFCHKPCPPGERKARDCTVNGDEPDCVPCQEGKEYTDKAHFSSK
CRRCRLCDEGHGLEVEINCTRTQNTKCRCKPNFFCNSTVCEHCDPCTKCE
HGIIKECTLTSNTKCKEEGSRSNLGWLCLLLLPIPLIVWVKRKEVQKTCR
KHRKENQGSHESPTLNPETVAINLSDVDLSKYITTIAGVMTLSQVKGFVR
KNGVNEAKIVEIKNDNVQDTAEQKVQLLRNWHQLHGKKEAYDTLIKDLKK
ANLCTLAEKIQTIILKDITSDSENSNFRNEIQSLV

An exemplary nucleotide sequence encoding the amino acid sequence of SEQ ID NO: 14 is set forth in SEQ ID NO: 15, which is provided below.

(SEQ ID NO: 15)
ATGCTGGGCATCTGGACCCTCCTACCTCTGGTTCTTACGTCTGTTGCTAG
ATTATCGTCCAAAAGTGTTAATGCCCAAGTGACTGACATCAACTCCAAGG
GATTGGAATTGAGGAAGACTGTTACTACAGTTGAGACTCAGAACTTGGAA
GGCCTGCATCATGATGGCCAATTCTGCCATAAGCCCTGTCCTCCAGGTGA
AAGGAAAGCTAGGGACTGCACAGTCAATGGGGATGAACCAGACTGCGTGC
CCTGCCAAGAAGGGAAGGAGTACACAGACAAAGCCCATTTTTCTTCCAAA
TGCAGAAGATGTAGATTGTGTGATGAAGGACATGGCTTAGAAGTGGAAAT
AAACTGCACCCGGACCCAGAATACCAAGTGCAGATGTAAACCAAACTTTT
TTTGTAACTCTACTGTATGTGAACACTGTGACCCTTGCACCAAATGTGAA
CATGGAATCATCAAGGAATGCACACTCACCAGCAACACCAAGTGCAAAGA
GGAAGGATCCAGATCTAACTTGGGGTGGCTTTGTCTTCTTCTTTTGCCAA
TTCCACTAATTGTTTGGGTGAAGAGAAAGGAAGTACAGAAAACATGCAGA
AAGCACAGAAAGGAAAACCAAGGTTCTCATGAATCTCCAACCTTAAATCC
TGAAACAGTGGCAATAAATTTATCTGATGTTGACTTGAGTAAATATATCA
CCACTATTGCTGGAGTCATGACACTAAGTCAAGTTAAAGGCTTTGTTCGA
AAGAATGGTGTCAATGAAGCCAAAATAGTTGAGATCAAGAATGACAATGT
CCAAGACACAGCAGAACAGAAAGTTCAACTGCTTCGTAATTGGCATCAAC
TTCATGGAAAGAAAGAAGCGTATGACACATTGATTAAAGATCTCAAAAAA
GCCAATCTTTGTACTCTTGCAGAGAAAATTCAGACTATCATCCTCAAGGA
CATTACTAGTGACTCAGAAAATTCAAACTTCAGAAATGAAATCCAAAGCT
TGGTC

Second Modification

The second modification is in the N-terminal region of a human Fas polypeptide.

In certain embodiments, the second modification is located between the peptide signal region and the cysteine rich domain 1 of Fas (e.g., human Fas). In certain embodiments, the peptide signal region of human Fas is encoded by amino acids 1 to 25 of human Fas. In certain embodiments, the peptide signal region of human Fas is encoded by amino acids 1 to 25 of SEQ ID NO: 10. In certain embodiments, the cysteine rich domain 1 of human Fas is encoded by amino acids 48 to 82 of human Fas. In certain embodiments, the cysteine rich domain 1 of human Fas is encoded by amino acids 48 to 82 of SEQ ID NO: 10. In certain embodiments, the second modification is within amino acids 26 to 47 of human Fas. In certain embodiments, the second modification is within amino acids 26 to 47 of SEQ ID NO: 10.

In certain embodiments, the second modification is selected from the group consisting of substitutions, deletions, and insertions. In certain embodiments, the substitution is a point mutation. In certain embodiments, the second modification is within amino acids 26 to 36 of SEQ ID NO: 10. In certain embodiments, the second modification is within amino acids 26 to 35 of SEQ ID NO: 10. In certain embodiments, the second modification comprises one or two point mutations or a deletion of a single amino acid within amino acids 26 to 36 of SEQ ID NO: 10. In certain embodiments, the second modification comprises one or two point mutations or a deletion of a single amino acid within amino acids 26 to 35 of SEQ ID NO: 10. In certain embodiments, the second modification increases the surface expression of the dominant negative Fas polypeptide by a cell, and/or increases the transduction efficiency of the dominant negative Fas polypeptide into a cell. In certain embodiments, the second modification increases the protection of the dominant negative Fas polypeptide from FasL-induced apoptosis. In certain embodiments, the protection conferred by the dominant negative Fas polypeptide is measured by the survival rate of cells expressing the dominant negative Fas polypeptide post FasL stimulation.

In certain embodiments, the second modification comprises or consists of a modification at position 32 of a human dominant negative Fas polypeptide (e.g., one comprising or consisting of the amino acid sequence set forth in SEQ ID NO: 12 or SEQ ID NO: 14). In certain embodiments, the second modification comprises or consists of a modification at position 33 of a human dominant negative Fas polypeptide (e.g., one comprising or consisting of the amino acid sequence set forth in SEQ ID NO: 12 or SEQ ID NO: 14).

In certain embodiments, the second modification comprises or consists of a deletion. In certain embodiments, the second modification comprises or consists of a deletion of up to one, up to two, up to three, up to four, or up to five amino acids. In certain embodiments, the second modification comprises or consists of a deletion of one amino acid. In certain embodiments, the second modification comprises or consists of a deletion of the amino acid at position 32. In certain embodiments, the second modification comprises or consists of a deletion of the amino acid at position 33.

In certain embodiments, the second modification consists of a deletion of the amino acid at position 32. In certain embodiments, the deletion consists of a deletion of amino acid 32 of a human dominant negative Fas polypeptide (e.g., a human dominant negative Fas polypeptide comprising or consisting of the amino acid sequence set forth in SEQ ID NO: 12 or SEQ ID NO: 14).

In certain embodiments, the first modification consists of point mutation D260V of human Fas and the second modification consists of a deletion of the amino acid 32 of human Fas.

In certain embodiments, the first modification consists of a deletion of amino acids 230-314 of human Fas and the second modification consists of a deletion of amino acid 32 of human Fas. In certain embodiments, the dominant negative Fas polypeptide comprises or consists of a first mortification consisting of a deletion of amino acid at positions 32 and a second mortification consisting of a deletion of amino acids 230-314 of a human wild-type Fas polypeptide consisting of the amino acid sequence set forth in SEQ ID NO: 10. In certain embodiments, the dominant negative Fas polypeptide is designated as “Fas del S32 DNR”. In certain embodiments, the dominant negative Fas polypeptide comprises or consists of the amino acid sequence set forth in SEQ ID NO: 16, which is provided below.

(SEQ ID NO: 16)
MLGIWTLLPLVLTSVARLSSKSVNAQVTDINKGLELRKTVTTVETQNLEG
LHHDGQFCHKPCPPGERKARDCTVNGDEPDCVPCQEGKEYTDKAHFSSKC
RRCRLCDEGHGLEVEINCTRTQNTKCRCKPNFFCNSTVCEHCDPCTKCEH
GIIKECTLTSNTKCKEEGSRSNLGWLCLLLLPIPLIVWVKRKEVQKTCRK
HRKENQGSHESPTLNPETVAINLSDVDLLKDITSDSENSNFRNEIQSLV

An exemplary nucleotide sequence encoding the amino acid sequence of SEQ ID NO: 16 is set forth in SEQ ID NO: 17, which is provided below.

(SEQ ID NO: 17)
ATGCTGGGCATCTGGACCCTCCTACCTCTGGTTCTTACGTCTGTTGCTAG
ATTATCGTCCAAAAGTGTTAATGCCCAAGTGACTGACATCAACAAGGGAT
TGGAATTGAGGAAGACTGTTACTACAGTTGAGACTCAGAACTTGGAAGGC
CTGCATCATGATGGCCAATTCTGCCATAAGCCCTGTCCTCCAGGTGAAAG
GAAAGCTAGGGACTGCACAGTCAATGGGGATGAACCAGACTGCGTGCCCT
GCCAAGAAGGGAAGGAGTACACAGACAAAGCCCATTTTTCTTCCAAATGC
AGAAGATGTAGATTGTGTGATGAAGGACATGGCTTAGAAGTGGAAATAAA
CTGCACCCGGACCCAGAATACCAAGTGCAGATGTAAACCAAACTTTTTTT
GTAACTCTACTGTATGTGAACACTGTGACCCTTGCACCAAATGTGAACAT
GGAATCATCAAGGAATGCACACTCACCAGCAACACCAAGTGCAAAGAGGA
AGGTTCCAGATCTAACTTGGGGTGGCTTTGTCTTCTTCTTTTGCCAATTC
CACTAATTGTTTGGGTGAAGAGAAAGGAAGTACAGAAAACATGCAGAAAG
CACAGAAAGGAAAACCAAGGTTCTCATGAATCTCCAACCTTAAATCCTGA
AACAGTGGCAATAAATTTATCTGATGTTGACTTGCTCAAGGACATTACTA
GTGACTCAGAAAATTCAAACTTCAGAAATGAAATCCAAAGCTTGGTC

In certain embodiments, the dominant negative Fas polypeptide comprises or consists of an amino acid sequence that is at least about 80%, at least about 85%, at least about 90%, at least about 95%, at least about 96%, at least about 97%, at least about 98%, at least about 99%, or at least about 100% homologous or identical to the amino acid sequence set forth in SEQ ID NO: 16. In certain embodiments, the dominant negative Fas polypeptide comprising or consisting of an amino acid sequence that is at least about 80%, at least about 85%, at least about 90%, at least about 95%, at least about 96%, at least about 97%, at least about 98%, at least about 99%, or at least about 100% homologous or identical to the amino acid sequence set forth in SEQ ID NO: 16 comprises or consists of a first modification consisting of a deletion of amino acids 230-314 and a second modification consisting of a deletion of amino acid 32 of a human wild-type Fas polypeptide (e.g., one comprising or consisting of the amino acid sequence set forth in SEQ ID NO: 10).

In certain embodiments, the second modification comprises or consists of a deletion of two amino acids. In certain embodiments, the second modification comprises or consists of a deletion of the amino acid at position 32. In certain embodiments, the second modification comprises or consists of a deletion of the two amino acids at positions 31 and 32.

In certain embodiments, the second modification consists of a deletion of the amino acids at positions 31 and 32. In certain embodiments, the deletion is a deletion of amino acids 31 and 32 of a human dominant negative Fas polypeptide (e.g., a human dominant negative Fas polypeptide comprising or consisting of the amino acid sequence set forth in SEQ ID NO: 12 or SEQ ID NO: 14).

In certain embodiments, the first modification consists of a point mutation D260V in human Fas and the second modification consists of a deletion of amino acids 31 and 32 of human Fas.

In certain embodiments, the first modification consists of a deletion of amino acids 230-314 of human Fas and the second modification consists of a deletion of amino acids 31 and 32 of human Fas. In certain embodiments, the dominant negative Fas polypeptide comprises or consists of a first modification consisting of a deletion of amino acids 230-314 and a second modification consisting of a deletion of amino acids 31 and 32 of a human wild-type Fas polypeptide consisting of the amino acid sequence set forth in SEQ ID NO: 10. In certain embodiments, the dominant negative Fas polypeptide is designated as “Fas del N31S32 DNR”, “FasDNR del31-32” or “Fas del31-32 DNR”. In certain embodiments, the dominant negative Fas polypeptide comprises or consists of the amino acid sequence set forth in SEQ ID NO: 18, which is provided below.

(SEQ ID NO: 18)
MLGIWTLLPLVLTSVARLSSKSVNAQVTDIKGLELRKTVTTVETQNLEGL
HHDGQFCHKPCPPGERKARDCTVNGDEPDCVPCQEGKEYTDKAHFSSKCR
RCRLCDEGHGLEVEINCTRTQNTKCRCKPNFFCNSTVCEHCDPCTKCEHG
IIKECTLTSNTKCKEEGSRSNLGWLCLLLLPIPLIVWVKRKEVQKTCRKH
RKENQGSHESPTLNPETVAINLSDVDLLKDITSDSENSNFRNEIQSLV

An exemplary nucleotide sequence encoding the amino acid sequence of SEQ ID NO: 18 is set forth in SEQ ID NO: 19, which is provided below.

(SEQ ID NO: 19)
ATGCTGGGCATCTGGACCCTCCTACCTCTGGTTCTTACGTCTGTTGCTAG
ATTATCGTCCAAAAGTGTTAATGCCCAAGTGACTGACATCAAGGGATTGG
AATTGAGGAAGACTGTTACTACAGTTGAGACTCAGAACTTGGAAGGCCTG
CATCATGATGGCCAATTCTGCCATAAGCCCTGTCCTCCAGGTGAAAGGAA
AGCTAGGGACTGCACAGTCAATGGGGATGAACCAGACTGCGTGCCCTGCC
AAGAAGGGAAGGAGTACACAGACAAAGCCCATTTTTCTTCCAAATGCAGA
AGATGTAGATTGTGTGATGAAGGACATGGCTTAGAAGTGGAAATAAACTG
CACCCGGACCCAGAATACCAAGTGCAGATGTAAACCAAACTTTTTTTGTA
ACTCTACTGTATGTGAACACTGTGACCCTTGCACCAAATGTGAACATGGA
ATCATCAAGGAATGCACACTCACCAGCAACACCAAGTGCAAAGAGGAAGG
TTCCAGATCTAACTTGGGGTGGCTTTGTCTTCTTCTTTTGCCAATTCCAC
TAATTGTTTGGGTGAAGAGAAAGGAAGTACAGAAAACATGCAGAAAGCAC
AGAAAGGAAAACCAAGGTTCTCATGAATCTCCAACCTTAAATCCTGAAAC
AGTGGCAATAAATTTATCTGATGTTGACTTGCTCAAGGACATTACTAGTG
ACTCAGAAAATTCAAACTTCAGAAATGAAATCCAAAGCTTGGTC

In certain embodiments, the dominant negative Fas polypeptide comprises or consists of an amino acid sequence that is at least about 80%, at least about 85%, at least about 90%, at least about 95%, at least about 96%, at least about 97%, at least about 98%, at least about 99%, or at least about 100% homologous or identical to the amino acid sequence set forth in SEQ ID NO: 18. In certain embodiments, the dominant negative Fas polypeptide comprising or consisting of an amino acid sequence that is at least about 80%, at least about 85%, at least about 90%, at least about 95%, at least about 96%, at least about 97%, at least about 98%, at least about 99%, or at least about 100% homologous or identical to the amino acid sequence set forth in SEQ ID NO: 18 comprises or consists of a first modification consisting of a deletion of amino acids 230-314 and a second modification consisting of a deletion of amino acids 31 and 32 of a human Fas polypeptide (e.g., one comprising or consisting of the amino acid sequence set forth in SEQ ID NO: 10).

In certain embodiments, the second modification comprises or consists of a deletion of the amino acids at positions 32 and 33. In certain embodiments, the deletion is a deletion of amino acids 32 and 33 of a human dominant negative Fas polypeptide (e.g., a human dominant negative Fas polypeptide comprising or consisting of the amino acid sequence set forth in SEQ ID NO: 12 or SEQ ID NO: 14).

In certain embodiments, the first modification consists of a point mutation D260V of human Fas and the second modification consists of a deletion of amino acids 32 and 33 of human Fas.

In certain embodiments, the first modification consists of a deletion of amino acids 230-314 of human Fas and the second modification consists of a deletion of amino acids 32 and 33 of human Fas. In certain embodiments, the dominant negative Fas polypeptide comprises or consists of a first modification consisting of a deletion of amino acids 230-314 and a second modification consisting of a deletion of amino acids 32 and 33 of a human wild-type Fas polypeptide consisting of the amino acid sequence set forth in SEQ ID NO: 10. In certain embodiments, the dominant negative Fas polypeptide is designated as “Fas del S32K33 DNR”. In certain embodiments, the dominant negative Fas polypeptide comprises or consists of the amino acid sequence set forth in SEQ ID NO: 20, which is provided below.

(SEQ ID NO: 20)
MLGIWTLLPLVLTSVARLSSKSVNAQVTDINGLELRKTVTTVETQNLEGL
HHDGQFCHKPCPPGERKARDCTVNGDEPDCVPCQEGKEYTDKAHFSSKCR
RCRLCDEGHGLEVEINCTRTQNTKCRCKPNFFCNSTVCEHCDPCTKCEHG
IIKECTLTSNTKCKEEGSRSNLGWLCLLLLPIPLIVWVKRKEVQKTCRKH
RKENQGSHESPTLNPETVAINLSDVDLLKDITSDSENSNFRNEIQSLV

An exemplary nucleotide sequence encoding the amino acid sequence of SEQ ID NO: 20 is set forth in SEQ ID NO: 21, which is provided below.

(SEQ ID NO: 21)
ATGCTGGGCATCTGGACCCTCCTACCTCTGGTTCTTACGTCTGTTGCTAG
ATTATCGTCCAAAAGTGTTAATGCCCAAGTGACTGACATCAACGGATTGG
AATTGAGGAAGACTGTTACTACAGTTGAGACTCAGAACTTGGAAGGCCTG
CATCATGATGGCCAATTCTGCCATAAGCCCTGTCCTCCAGGTGAAAGGAA
AGCTAGGGACTGCACAGTCAATGGGGATGAACCAGACTGCGTGCCCTGCC
AAGAAGGGAAGGAGTACACAGACAAAGCCCATTTTTCTTCCAAATGCAGA
AGATGTAGATTGTGTGATGAAGGACATGGCTTAGAAGTGGAAATAAACTG
CACCCGGACCCAGAATACCAAGTGCAGATGTAAACCAAACTTTTTTTGTA
ACTCTACTGTATGTGAACACTGTGACCCTTGCACCAAATGTGAACATGGA
ATCATCAAGGAATGCACACTCACCAGCAACACCAAGTGCAAAGAGGAAGG
TTCCAGATCTAACTTGGGGTGGCTTTGTCTTCTTCTTTTGCCAATTCCAC
TAATTGTTTGGGTGAAGAGAAAGGAAGTACAGAAAACATGCAGAAAGCAC
AGAAAGGAAAACCAAGGTTCTCATGAATCTCCAACCTTAAATCCTGAAAC
AGTGGCAATAAATTTATCTGATGTTGACTTGCTCAAGGACATTACTAGTG
ACTCAGAAAATTCAAACTTCAGAAATGAAATCCAAAGCTTGGTC

In certain embodiments, the dominant negative Fas polypeptide comprises or consists of an amino acid sequence that is at least about 80%, at least about 85%, at least about 90%, at least about 95%, at least about 96%, at least about 97%, at least about 98%, at least about 99%, or at least about 100% homologous or identical to the amino acid sequence set forth in SEQ ID NO: 20. In certain embodiments, the dominant negative Fas polypeptide comprising or consisting of an amino acid sequence that is at least about 80%, at least about 85%, at least about 90%, at least about 95%, at least about 96%, at least about 97%, at least about 98%, at least about 99%, or at least about 100% homologous or identical to the amino acid sequence set forth in SEQ ID NO: 20 comprises or consists of a first modification consisting of a deletion of amino acids 230-314 and a second modification consisting of a deletion of amino acids 32 and 33 of a human Fas polypeptide (e.g., one comprising or consisting of the amino acid sequence set forth in SEQ ID NO: 10).

In certain embodiments, the second modification comprises or consists of a deletion of the amino acid at position 33. In certain embodiments, the second modification comprises or consists of a deletion of the amino acids at positions 33 and 34. In certain embodiments, the deletion comprises or consists of a deletion of amino acids 33 and 34 of a human dominant negative Fas polypeptide (e.g., a human dominant negative Fas polypeptide comprising or consisting of the amino acid sequence set forth in SEQ ID NO: 12 or SEQ ID NO: 14).

In certain embodiments, the first modification consists of a point mutation D260V of human Fas and the second modification consists of a deletion of amino acids 33 and 34 of human Fas.

In certain embodiments, the first modification consists of a deletion of amino acids 230-314 of human Fas and the second modification consists of a deletion of amino acids 33 and 34 of human Fas. In certain embodiments, the dominant negative Fas polypeptide comprises or consists of a first modification consisting of a deletion of amino acids 230-314 and a second modification consisting of a deletion of amino acids 33 and 34 and of a human wild-type Fas polypeptide consisting of the amino acid sequence set forth in SEQ ID NO: 10. In certain embodiments, the dominant negative Fas polypeptide is designated as “Fas del K33G34 DNR”. In certain embodiments, the dominant negative Fas polypeptide comprises or consists of the amino acid sequence set forth in SEQ ID NO: 22, which is provided below.

(SEQ ID NO: 22)
MLGIWTLLPLVLTSVARLSSKSVNAQVTDINSLELRKTVTTVETQNLEGL
HHDGQFCHKPCPPGERKARDCTVNGDEPDCVPCQEGKEYTDKAHFSSKCR
RCRLCDEGHGLEVEINCTRTQNTKCRCKPNFFCNSTVCEHCDPCTKCEHG
IIKECTLTSNTKCKEEGSRSNLGWLCLLLLPIPLIVWVKRKEVQKTCRKH
RKENQGSHESPTLNPETVAINLSDVDLLKDITSDSENSNFRNEIQSLV

An exemplary nucleotide sequence encoding the amino acid sequence of SEQ ID NO: 22 is set forth in SEQ ID NO: 23, which is provided below.

(SEQ ID NO: 23)
ATGCTGGGCATCTGGACCCTCCTACCTCTGGTTCTTACGTCTGTTGCTAG
ATTATCGTCCAAAAGTGTTAATGCCCAAGTGACTGACATCAACTCATTGG
AATTGAGGAAGACTGTTACTACAGTTGAGACTCAGAACTTGGAAGGCCTG
CATCATGATGGCCAATTCTGCCATAAGCCCTGTCCTCCAGGTGAAAGGAA
AGCTAGGGACTGCACAGTCAATGGGGATGAACCAGACTGCGTGCCCTGCC
AAGAAGGGAAGGAGTACACAGACAAAGCCCATTTTTCTTCCAAATGCAGA
AGATGTAGATTGTGTGATGAAGGACATGGCTTAGAAGTGGAAATAAACTG
CACCCGGACCCAGAATACCAAGTGCAGATGTAAACCAAACTTTTTTTGTA
ACTCTACTGTATGTGAACACTGTGACCCTTGCACCAAATGTGAACATGGA
ATCATCAAGGAATGCACACTCACCAGCAACACCAAGTGCAAAGAGGAAGG
TTCCAGATCTAACTTGGGGTGGCTTTGTCTTCTTCTTTTGCCAATTCCAC
TAATTGTTTGGGTGAAGAGAAAGGAAGTACAGAAAACATGCAGAAAGCAC
AGAAAGGAAAACCAAGGTTCTCATGAATCTCCAACCTTAAATCCTGAAAC
AGTGGCAATAAATTTATCTGATGTTGACTTGCTCAAGGACATTACTAGTG
AACTCAGAAAATTCAACTTCAGAAATGAAATCCAAAGCTTGGTC

In certain embodiments, the dominant negative Fas polypeptide comprises or consists of an amino acid sequence that is at least about 80%, at least about 85%, at least about 90%, at least about 95%, at least about 96%, at least about 97%, at least about 98%, at least about 99%, or at least about 100% homologous or identical to the amino acid sequence set forth in SEQ ID NO: 22. In certain embodiments, the dominant negative Fas polypeptide comprising or consisting of an amino acid sequence that is at least about 80%, at least about 85%, at least about 90%, at least about 95%, at least about 96%, at least about 97%, at least about 98%, at least about 99%, or at least about 100% homologous or identical to the amino acid sequence set forth in SEQ ID NO: 22 comprises or consists of a first modification consisting of a deletion of amino acids 230-314 and a second modification consisting of a deletion of amino acids 33 and 34 of a human Fas polypeptide (e.g., one comprising or consisting of the amino acid sequence set forth in SEQ ID NO: 10).

In certain embodiments, the second modification consists of a point mutation. In certain embodiments, the point mutation is at position 32 of a human dominant negative Fas polypeptide (e.g., a human dominant negative Fas polypeptide comprising or consisting of the amino acid sequence set forth in SEQ ID NO: 12 or SEQ ID NO: 14). In certain embodiments, the point mutation is S32A.

In certain embodiments, the first modification consists of a point mutation D260V of human Fas and the second modification consists of a point mutation S32A of human Fas.

In certain embodiments, the first modification consists of a deletion of amino acids 230-314 of human Fas and the second modification consists of a point mutation S32A of human Fas. In certain embodiments, the dominant negative Fas polypeptide comprises or consists of a first modification consisting of a deletion of amino acids 230-314 and a second modification consisting of a point mutation S32A of a human wild-type Fas polypeptide consisting of the amino acid sequence set forth in SEQ ID NO: 10. In certain embodiments, the dominant negative Fas polypeptide is designated as “Fas S32A DNR”. In certain embodiments, the dominant negative Fas polypeptide comprises or consists of the amino acid sequence set forth in SEQ ID NO: 24. SEQ ID NO: 24 is provided below.

(SEQ ID NO: 24)
MLGIWTLLPLVLTSVARLSSKSVNAQVTDINAKGLELRKTVTTVETQNLE
GLHHDGQFCHKPCPPGERKARDCTVNGDEPDCVPCQEGKEYTDKAHFSSK
CRRCRLCDEGHGLEVEINCTRTQNTKCRCKPNFFCNSTVCEHCDPCTKCE
HGIIKECTLTSNTKCKEEGSRSNLGWLCLLLLPIPLIVWVKRKEVQKTCR
KHRKENQGSHESPTLNPETVAINLSDVDLLKDITSDSENSNFRNEIQSLV

An exemplary nucleotide sequence encoding the amino acid sequence of SEQ ID NO: 24 is set forth in SEQ ID NO: 25, which is provided below.

(SEQ ID NO: 25)
ATGCTGGGCATCTGGACCCTCCTACCTCTGGTTCTTACGTCTGTTGCTAG
ATTATCGTCCAAAAGTGTTAATGCCCAAGTGACTGACATCAACGCCAAGG
GATTGGAATTGAGGAAGACTGTTACTACAGTTGAGACTCAGAACTTGGAA
GGCCTGCATCATGATGGCCAATTCTGCCATAAGCCCTGTCCTCCAGGTGA
AAGGAAAGCTAGGGACTGCACAGTCAATGGGGATGAACCAGACTGCGTGC
CCTGCCAAGAAGGGAAGGAGTACACAGACAAAGCCCATTTTTCTTCCAAA
TGCAGAAGATGTAGATTGTGTGATGAAGGACATGGCTTAGAAGTGGAAAT
AAACTGCACCCGGACCCAGAATACCAAGTGCAGATGTAAACCAAACTTTT
TTTGTAACTCTACTGTATGTGAACACTGTGACCCTTGCACCAAATGTGAA
CATGGAATCATCAAGGAATGCACACTCACCAGCAACACCAAGTGCAAAGA
GGAAGGTTCCAGATCTAACTTGGGGTGGCTTTGTCTTCTTCTTTTGCCAA
TTCCACTAATTGTTTGGGTGAAGAGAAAGGAAGTACAGAAAACATGCAGA
AAGCACAGAAAGGAAAACCAAGGTTCTCATGAATCTCCAACCTTAAATCC
TGAAACAGTGGCAATAAATTTATCTGATGTTGACTTGCTCAAGGACATTA
CTAGTGACTCAGAAAATTCAAACTTCAGAAATGAAATCCAAAGCTTGGTC

In certain embodiments, the dominant negative Fas polypeptide comprises or consists of an amino acid sequence that is at least about 80%, at least about 85%, at least about 90%, at least about 95%, at least about 96%, at least about 97%, at least about 98%, at least about 99%, or at least about 100% homologous or identical to the amino acid sequence set forth in SEQ ID NO: 24. In certain embodiments, the dominant negative Fas polypeptide comprising or consisting of an amino acid sequence that is at least about 80%, at least about 85%, at least about 90%, at least about 95%, at least about 96%, at least about 97%, at least about 98%, at least about 99%, or at least about 100% homologous or identical to the amino acid sequence set forth in SEQ ID NO: 24 comprises or consists of a first modification consisting of a deletion of amino acids 230-314 and a second modification consisting of a point mutation S32A of a human Fas polypeptide (e.g., one comprising or consisting of the amino acid sequence set forth in SEQ ID NO: 10).

In certain embodiments, the dominant negative Fas polypeptide comprises a heterologous signal peptide, for example, an IL-2 signal peptide, a kappa leader sequence, a CD8 leader sequence or a peptide with essentially equivalent activity.

### 3. Antigen-Recognizing Receptors

The present disclosure provides antigen-recognizing receptors that bind to an antigen. In certain embodiments, the antigen-recognizing receptor is a chimeric antigen receptor (CAR). In certain embodiments, the antigen-recognizing receptor is a T-cell receptor (TCR). The antigen-recognizing receptor can bind to a tumor antigen or a pathogen antigen. In certain embodiments, the antigen-recognizing receptor binds to a tumor antigen. In certain embodiments, the tumor antigen is a tumor-specific antigen or a tumor-associated antigen.

3.1. Antigens

In certain embodiments, the antigen-recognizing receptor binds to a tumor antigen. Any tumor antigen (antigenic peptide) can be used in the tumor-related embodiments described herein. Sources of antigen include, but are not limited to, cancer proteins. The antigen can be expressed as a peptide or as an intact protein or portion thereof. The intact protein or a portion thereof can be native or mutagenized. In certain embodiments, the tumor antigen is a tumor specific antigen (TSA). In certain embodiment, the tumor antigen is a tumor-associated antigen (TAA).

Non-limiting examples of tumor antigens include CD19, MUC16, MUC1, CAIX, CEA, CD8, CD7, CD10, CD20, CD22, CD30, CLL1, CD33, CD34, CD38, CD41, CD44, CD49f, CD56, CD74, CD133, CD138, EGP-2, EGP-40, EpCAM, Erb-B2, Erb-B3, Erb-B4, FBP, Fetal acetylcholine receptor, folate receptor-α, GD2, GD3, HER-2, hTERT, IL-13R-α2, κ-light chain, KDR, mutant KRAS (including, but not limited to, G12V, G12D, G12C), mutant HRAS, mutant PIK3CA (including, but not limited to, E52K, E545K, H1047R, H1047L), mutant IDH (including, but not limited to, R132H), mutant p53 (including, but not limited to, R175H, Y220C, G245D, G245S, R248L, R248Q, R248W, R249S, R273C, R273L, R273H and R282W), mutant NRAS (including, but not limited to, Q61R, Q61K, and Q61L), LeY, L1 cell adhesion molecule, MAGE-A1, Mesothelin, ERBB2, MAGEA3, CT83 (also known as KK-LC-1), p53, MART1, GP100, Proteinase3 (PR1), Tyrosinase, Survivin, hTERT, EphA2, NKG2D ligands, NY-ESO-1, oncofetal antigen (h5T4), PSCA, PSMA, ROR1, TAG-72, VEGF-R2, WT-1, BCMA, CD123, CD44V6, NKCS1, EGF1R, EGFR-VIII, CD99, CD70, ADGRE2, CCR1, LILRB2, PRAME, HPV E6 oncoprotein, HPV E7 oncoprotein, and ERBB. In certain embodiments, the tumor antigen is CD19.

In certain embodiments, the antigen-recognizing receptor binds to a human CD19 polypeptide. In certain embodiments, the human CD19 polypeptide comprises or consists of the amino acid sequence set forth in SEQ ID NO: 26 or a fragment thereof. SEQ ID NO: 26 is provided below.

[SEQ ID NO: 26]
PEEPLVVKVEEGDNAVLQCLKGTSDGPTQQLTWSRESPLKPFLKLSLGLP
GLGIHMRPLAIWLFIFNVSQQMGGFYLCQPGPPSEKAWQPGWTVNVEGSG
ELFRWNVSDLGGLGCGLKNRSSEGPSSPSGKLMSPKLYVWAKDRPEIWEG
EPPCLPPRDSLNQSLSQDLTMAPGSTLWLSCGVPPDSVSRGPLSWTHVHP
KGPKSLLSLELKDDRPARDMWVMETGLLLPRATAQDAGKYYCHRGNLTMS
FHLEITARPVLWHWLLRTGGWK

In certain embodiments, the antigen-recognizing receptor binds to the extracellular domain of a human CD19 protein.

In certain embodiments, the antigen-recognizing receptor binds to a pathogen antigen, e.g., for use in treating and/or preventing a pathogen infection. Non-limiting examples of pathogens include viruses, bacteria, fungi, parasites, and protozoans capable of causing disease.

Non-limiting examples of pathogenic viruses include, Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Naira viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses), human papilloma virus (i.e. HPV), JC virus, Epstein Bar Virus, Merkel cell polyoma virus.

Non-limiting examples of pathogenic bacteria include Pasteurella, Staphylococci, Streptococcus, Escherichia coli, Pseudomonas species, and Salmonella species. Specific examples of infectious bacteria include but are not limited to, Helicobacter pyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, Corynebacterium diphtherias, corynebacterium sp., Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, Rickettsia, Clostridium difficile, and Actinomyces israelli.

In certain embodiments, the pathogen antigen is a viral antigen present in Cytomegalovirus (CMV), a viral antigen present in Epstein Barr Virus (EBV), a viral antigen present in Human Immunodeficiency Virus (HIV), or a viral antigen present in influenza virus.

3.2. T-Cell Receptor (TCR)

In certain embodiments, the antigen-recognizing receptor is a TCR. A TCR is a disulfide-linked heterodimeric protein consisting of two variable chains expressed as part of a complex with the invariant CD3 chain molecules. A TCR is found on the surface of T cells, and is responsible for recognizing antigens as peptides bound to major histocompatibility complex (MHC) molecules. In certain embodiments, a TCR comprises an alpha chain and a beta chain (encoded by TRA and TRB, respectively). In certain embodiments, a TCR comprises a gamma chain and a delta chain (encoded by TRG and TRD, respectively).

Each chain of a TCR is composed of two extracellular domains comprising a Variable (V) region and a Constant (C) region. The Constant region is proximal to the cell membrane, followed by a transmembrane region and a short cytoplasmic tail that lacks the ability to transduce a signal. The Variable region binds to the peptide/MHC complex. The variable domain of each pair (alpha/beta or gamma/delta) of TCR polypeptides comprises three complementarity determining regions (CDRs).

In certain embodiments, a TCR can form a receptor complex with three dimeric signaling modules CD3δ/ε, CD3γ/ε and CD3ζ/ζ or ζ/η. When a TCR complex engages with its antigen and MHC (peptide/MHC), the T cell expressing the TCR complex is activated.

In certain embodiments, the TCR is an endogenous TCR. In certain embodiments, the TCR recognizes a viral antigen. In certain embodiments, the TCR is expressed in a virus-specific T cell. In certain embodiments, the virus-specific T cell is derived from an individual immune to a viral infection, e.g., BK virus, human herpesvirus 6, Epstein-Barr virus (EBV), cytomegalovirus or adenovirus. In certain embodiments, the virus-specific T cell is a T cell disclosed in Leen et al., Blood, Vol. 121, No. 26, 2013; Barker et al., Blood, Vol. 116, No. 23, 2010; Tzannou et al., Journal of Clinical Oncology, Vol. 35, No. 31, 2017; or Bollard et al., Blood, Vol. 32, No. 8, 2014, each of which is incorporated by reference in its entirety. In certain embodiments, the TCR recognizes a tumor antigen (including a TAA or TSA). In certain embodiments, the TCR is expressed in a tumor-specific T cell. In certain embodiments, the tumor-specific T cell is a tumor-infiltrating T cell generated by culturing T cells with explants of a tumor, e.g., melanoma or an epithelial cancer. In certain embodiments, the tumor-specific T cell is a T cell disclosed in Stevanovic et al, Science, 356, 200-205, 2017; Dudley et al. Journal of Immunotherapy, 26(4): 332-342, 2003; or Goff et al, Journal of Clinical Oncology, Vol. 34, No. 20, 2016, each of which is incorporated by reference in its entirety.

In certain embodiments, the antigen-recognizing receptor is a recombinant TCR. In certain embodiments, the recombinant TCR differs from any naturally occurring TCR by at least one amino acid residue. In certain embodiments, the non-naturally occurring TCR differs from any naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues. In certain embodiments, the non-naturally occurring TCR is modified from a naturally occurring TCR by at least one amino acid residue. In certain embodiments, the non-naturally occurring TCR is modified from a naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues.

3.3. Chimeric Antigen Receptor (CAR)

In certain embodiments, the antigen-recognizing receptor is a CAR. CARs are engineered receptors, which graft or confer a specificity of interest onto an immune effector cell or immunoresponsive cell. CARs can be used to confer non-MHC-restricted antigen specificity onto a T cell. Transfer of their coding sequence facilitated by retroviral vectors.

CARs have developed via a series of significant improvements referred to as “generations”. So-called “first generation” CARs are typically composed of an extracellular antigen-binding domain (e.g., a single chain variable fragment (scFv)), which is fused to a transmembrane domain, which is fused to a cytoplasmic/intracellular signaling domain. The cytoplasmic/intracellular signaling domain can comprise a single activating domain—usually an ITAM derived from CD3zeta. “First generation” CARs can provide de novo antigen recognition and cause activation of both CD4⁺ and CD8⁺ T cells through their CD3ζ chain signaling domain in a single fusion molecule, independent of HLA-mediated antigen presentation. “Second generation” CARs add an intracellular signaling domain derived from any one of the various co-stimulatory molecules (e.g., CD28, 4-1BB, ICOS, OX40) to the cytoplasmic tail of the CAR to provide additional signals to the T cell. “Second generation” CARs comprise those that provide both co-stimulation (e.g., CD28 or 4-1BB) and activation (CD3ζ). “Third generation” CARs comprise those that provide multiple co-stimulation (e.g., CD28 and 4-1BB) and activation (CD3ζ). In certain embodiments, the antigen-recognizing receptor is a first-generation CAR. In certain embodiments, the antigen-recognizing receptor is a second-generation CAR. In certain embodiments, the antigen-recognizing receptor is a third-generation CAR.

In certain embodiments, the extracellular antigen-binding domain of the CAR (embodied, for example, in an scFv or an analog thereof) binds to an antigen with a dissociation constant (K_{d}) of about 2×10⁻⁷ M or less. In certain embodiments, the K_{d} is about 2×10⁻⁷ M or less, about 1×10⁻⁷ M or less, about 9×10⁻⁸ M or less, about 1×10⁻⁸M or less, about 9×10⁻⁹M or less, about 5×10⁻⁹ M or less, about 4×10⁻⁹M or less, about 3×10⁻⁹ or less, about 2×10⁻⁹M or less, or about 1×10⁻⁹ M or less. In certain embodiments, the K_{d} is about 3×10⁻⁹M or less. In certain embodiments, the K_{d} is from about 1×10⁻⁹ M to about 3×10⁻⁷ M. In certain embodiments, the K_{d} is from about 1.5×10⁻⁹ M to about 3×10⁻⁷ M. In certain embodiments, the K_{d} is from about 1.5×10⁻⁹M to about 2.7×10⁻⁷ M.

Binding of the extracellular antigen-binding domain (for example, an scFv or an analog thereof) can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or an scFv) specific for the complex of interest. For example, the scFv can be radioactively labeled and used in a radioimmunoassay (MA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography. In certain embodiments, the extracellular antigen-binding domain of the CAR is labeled with a fluorescent marker. Non-limiting examples of fluorescent markers include green fluorescent protein (GFP), blue fluorescent protein (e.g., EBFP, EBFP2, Azurite, and mKalama1), cyan fluorescent protein (e.g., ECFP, Cerulean, and CyPet), and yellow fluorescent protein (e.g., YFP, Citrine, Venus, and YPet). Binding of the extracellular antigen-binding domain can also be confirmed by measuring the secretion of cytokines.

In accordance with the presently disclosed subject matter, a CAR comprises an extracellular antigen-binding domain, a transmembrane domain and an intracellular signaling domain, wherein the extracellular antigen-binding domain specifically binds to an antigen, which can be a tumor antigen (TAA or TSA) or a pathogen antigen.

In certain embodiments, the CAR comprises an extracellular antigen-binding domain that binds to CD19. In certain embodiments, the CAR is one described in Kochenderfer, J N et al. Blood. 2010 Nov. 11; 116(19):3875-86, which is incorporated herein by reference in its entirety.

3.3.1. Extracellular Antigen Binding Domain of a CAR

In certain embodiments, the extracellular antigen-binding domain specifically binds to an antigen. In certain embodiments, the antigen is a tumor antigen. In certain embodiments, the tumor antigen is a tumor specific antigen (TSA). In certain embodiments, the tumor antigen is a tumor-associated antigen (TAA). In certain embodiments, the tumor antigen is CD19. In certain embodiments, the extracellular antigen-binding domain is an scFv. In certain embodiments, the scFv is a human scFv. In certain embodiments, the scFv is a humanized scFv. In certain embodiments, the scFv is a murine scFv. In certain embodiments, the extracellular antigen-binding domain is a Fab, which is optionally crosslinked. In certain embodiments, the extracellular antigen-binding domain is a F(ab)₂. In certain embodiments, any of the foregoing molecules may be comprised in a fusion protein with a heterologous sequence to form the extracellular antigen-binding domain. In certain embodiments, the scFv is identified by screening scFv phage library with an antigen-Fc fusion protein. In certain embodiments, the antigen is a tumor antigen. In certain embodiments, the antigen is a pathogen antigen.

3.3.2. Transmembrane Domain of a CAR

In certain embodiments, the transmembrane domain of the CAR comprises a hydrophobic alpha helix that spans at least a portion of the membrane. Different transmembrane domains result in different receptor stability. After antigen recognition, receptors cluster and a signal is transduced to the cell. In accordance with the presently disclosed subject matter, the transmembrane domain of the CAR can comprise a CD8 polypeptide, a CD28 polypeptide, a CD3ζ polypeptide, a CD4 polypeptide, a 4-1BB polypeptide, an OX40 polypeptide, an ICOS polypeptide, a synthetic peptide (not based on a protein associated with the immune response), or a combination thereof.

In certain embodiments, the transmembrane domain comprises a CD8 polypeptide. In certain embodiments, the transmembrane domain comprises a transmembrane domain of human CD8 or a portion thereof. In certain embodiments, the CD8 polypeptide comprises or consists of an amino acid sequence that is at least about 85%, about 90%, about 95%, about 96%, about 97%, about 98%, about 99%, or about 100% homologous or identical to the sequence having a NCBI Reference No: NP_001139345.1 (SEQ ID NO: 27) or a fragment thereof, and/or may optionally comprise or consist of up to one or up to two or up to three conservative amino acid substitutions. In certain embodiments, the CD8 polypeptide comprises or consists of an amino acid sequence that is a consecutive portion of SEQ ID NO: 27 which is at least 20, or at least 30, or at least 40, or at least 50, and up to 235 amino acids in length. In certain embodiments, the CD8 polypeptide comprises or consists of an amino acid sequence of amino acids 1 to 235, 1 to 50, 50 to 100, 100 to 150, 137 to 209 150 to 200, or 200 to 235 of SEQ ID NO: 27. In certain embodiments, the CAR comprises a transmembrane domain of CD8 (e.g., human CD8) or a portion thereof. In certain embodiments, the transmembrane domain of the CAR comprises a CD8 polypeptide comprising or consisting of an amino acid sequence of amino acids 137 to 209 of SEQ ID NO: 27. SEQ ID NO: 27 is provided below.

[SEQ ID NO: 27]
MALPVTALLLPLALLLHAARPSQFRVSPLDRTWNLGETVELKCQVLLSNP
TSGCSWLFQPRGAAASPTFLLYLSQNKPKAAEGLDTQRFSGKRLGDTFVL
TLSDFRRENEGYYFCSALSNSIMYFSHFVPVFLPAKPTTTPAPRPPTPAP
TIASQPLSLRPEACRPAAGGAVHTRGLDFACDIYIWAPLAGTCGVLLLSL
VITLYCNHRNRRRVCKCPRPVVKSGDKPSLSARYV

In certain embodiments, the transmembrane domain comprises a transmembrane domain of murine CD8 or a portion thereof. In certain embodiments, the CD8 polypeptide comprises or consists of an amino acid sequence that is at least about 85%, about 90%, about 95%, about 96%, about 97%, about 98%, about 99% or about 100% homologous or identical to the sequence having a NCBI Reference No: AAA92533.1 (SEQ ID NO: 28) or a fragment thereof, and/or may optionally comprise or consist of up to one or up to two or up to three conservative amino acid substitutions. In certain embodiments, the CD8 polypeptide comprises or consists of an amino acid sequence that is a consecutive portion of SEQ ID NO: 28 which is at least about 20, or at least about 30, or at least about 40, or at least about 50, or at least about 60, or at least about 70, or at least about 100, or at least about 200, and up to 247 amino acids in length. In certain embodiments, the CD8 polypeptide comprises or consists of an amino acid sequence of amino acids 1 to 247, 1 to 50, 50 to 100, 100 to 150, 150 to 200, 151 to 219, or 200 to 247 of SEQ ID NO: 28. In certain embodiments, the transmembrane domain of the CAR comprises or consists of a CD8 polypeptide comprising or consisting of an amino acid sequence of amino acids 151 to 219 of SEQ ID NO: 28. SEQ ID NO: 28 is provided below.

| [SEQ ID NO: 28] | |
|---|---|
| 1 | MASPLTRFLS LNLLLMGESI ILGSGEAKPQ APELRIEPKK MDAELGQKVD LVCEVLGSVS |
| 61 | QGCSWLFONS SSKLPQPTFV VYMASSHNKI TWDEKLNSSK LFSAVRDTNN KYVLTLNKFS |
| 121 | KENEGYYFCS VISNSVMYFS SVVPVLQKVN STTTKPVLRT PSPVHPTGTS QPQRPEDCRP |
| 181 | RGSVKGTGLD FACDIYIWAP LAGICVAPLL SLIITLICYH RSRKRVCKCP RPLVRQEGKP |
| 241 | RPSEKIV |

In accordance with the presently disclosed subject matter, a “CD8 nucleic acid molecule” refers to a polynucleotide encoding a CD8 polypeptide.

In certain embodiments, the transmembrane domain of a presently disclosed CAR comprises a CD28 polypeptide. In certain embodiments, the transmembrane domain comprises a transmembrane domain of human CD28 or a portion thereof. In certain embodiments, the CD28 polypeptide comprises or consists of an amino acid sequence that is at least about 85%, about 90%, about 95%, about 96%, about 97%, about 98%, about 99% or 100% homologous or identical to the sequence having a NCBI Reference No: NP_006130 (SEQ ID No: 29) or a fragment thereof, and/or may optionally comprise up to one or up to two or up to three conservative amino acid substitutions. In certain embodiments, the CD28 polypeptide comprises or consists of an amino acid sequence that is a consecutive portion of SEQ ID NO: 29 which is at least 20, or at least 30, or at least 40, or at least 50, and up to 220 amino acids in length. In certain embodiments, the CD28 polypeptide comprises or consists of an amino acid sequence of amino acids 1 to 220, 1 to 50, 50 to 100, 100 to 150, 114 to 220, 150 to 200, 153 to 179, or 200 to 220 of SEQ ID NO: 29. In certain embodiments, the CD28 polypeptide comprises or consists of an amino acid sequence of amino acids 114 to 220 of SEQ ID NO: 29. In certain embodiments, the transmembrane domain of the CAR comprises a CD28 polypeptide comprising or consisting of amino acids 153 to 179 of SEQ ID NO: 29. SEQ ID NO: 29 is provided below:

| [SEQ ID NO: 29] | |
|---|---|
| 1 | MLRLLLALNL FPSIQVTGNK ILVKQSPMLV AYDNAVNLSC KYSYNLFSRE FRASLHKGLD |
| 61 | SAVEVCVVYG NYSQQLQVYS KTGFNCDGKL GNESVTFYLQ NLYVNQTDIY FCKIEVMYPP |
| 121 | PYLDNEKSNG TIIHVKGKHL CPSPLFPGPS KPFWVLVVVG GVLACYSLLV TVAFIIFWVR |
| 181 | SKRSRLLHSD YMNMTPRRPG PTRKHYQPYA PPRDFAAYRS |

An exemplary nucleic acid sequence encoding amino acids 153 to 179 of SEQ ID NO: 29 is set forth in SEQ ID NO: 30, which is provided below.

[SEQ ID NO: 30]
TTTTGGGTGCTGGTGGTGGTTGGTGGAGTCCTGGCTTGCTATAGCTTGCT
AGTAACAGTGGCCTTTATTATTTTCTGGGTG

In certain embodiments, the transmembrane domain of a presently disclosed CAR comprises a transmembrane domain of murine CD28 or a fragment thereof. In certain embodiments, the CD28 polypeptide comprises or consists of an amino acid sequence that is at least about 85%, about 90%, about 95%, about 96%, about 97%, about 98%, about 99% or 100% homologous or identical to the sequence having a NCBI Reference No: NP_031668.3 (SEQ ID No: 31) or a fragment thereof, and/or may optionally comprise or consist of up to one or up to two or up to three conservative amino acid substitutions. In certain embodiments, the CD28 polypeptide comprises or consists of an amino acid sequence that is a consecutive portion of SEQ ID NO: 31 which is at least 20, or at least 30, or at least 40, or at least 50, and up to 218 amino acids in length. In certain embodiments, the CD28 polypeptide comprises or consists of an amino acid sequence of amino acids 1 to 218, 1 to 50, 50 to 100, 100 to 150, 114 to 220, 150 to 200, 151 to 177, or 200 to 220 of SEQ ID NO: 31. In certain embodiments, the CD28 polypeptide comprises or consists of an amino acid sequence of amino acids 114 to 220 of SEQ ID NO: 31. In certain embodiments, the transmembrane domain of the CAR comprises or consists of a CD28 polypeptide comprising or consisting of amino acids 151 to 177 of SEQ ID NO: 31. SEQ ID NO: 31 is provided below:

| [SEQ ID NO: 31] | |
|---|---|
| 1 | MTLRLLFLAL NFFSVQVTEN KILVKQSPLL VVDSNEVSLS CRYSYNLLAK EFRASLYKGV |
| 61 | NSDVEVCVGN GNFTYQPQFR SNAEFNCDGD FDNETVTFRL WNLHVNHTDI YFCKIEFMYP |
| 121 | PPYLDNERSN GTIIHIKEKH LCHTQSSPKL FWALVVVAGV LFCYGLLVTV ALCVIWTNSR |
| 181 | RNRLLQSDYM NMTPRRPGLT RKPYQPYAPA RDFAAYRP |

In accordance with the presently disclosed subject matter, a “CD28 nucleic acid molecule” refers to a polynucleotide encoding a CD28 polypeptide.

In certain embodiments, the CAR further comprises a spacer region that links the extracellular antigen-binding domain to the transmembrane domain. The spacer region can be flexible enough to allow the antigen binding domain to orient in different directions to facilitate antigen recognition. The spacer region can be the hinge region from IgG1, or the CH2CH3 region of immunoglobulin and portions of CD3, a portion of a CD28 polypeptide (e.g., a portion of SEQ ID NO: 29 or SEQ ID NO: 31), a portion of a CD8 polypeptide (e.g., a portion of SEQ ID NO: 27, or a portion of SEQ ID NO: 28), a variation of any of the foregoing which is at least about 80%, at least about 85%, at least about 90%, or at least about 95% homologous or identical thereto, or a synthetic spacer sequence.

3.3.3. Intracellular Signaling Domain of a CAR

In certain embodiments, the intracellular signaling domain of the CAR comprises a CD3ζ polypeptide, which can activate or stimulate a cell (e.g., a cell of the lymphoid lineage, e.g., a T cell). Wild type (“native”) CD3ζ comprises three immunoreceptor tyrosine-based activation motifs (“ITAMs”) (e.g., ITAM1, ITAM2 and ITAM3), and transduces an activation signal to the cell (e.g., a cell of the lymphoid lineage, e.g., a T cell) after antigen is bound. The intracellular signaling domain of the native CD3ζ polypeptide is the primary transmitter of signals from endogenous TCRs.

In certain embodiments, the intracellular signaling domain of the CAR comprises a native CD3ζ polypeptide. In certain embodiments, the intracellular signaling domain of the CAR comprises a human CD3ζ polypeptide. In certain embodiments, the CD3ζ polypeptide comprises or consists of an amino acid sequence that is at least about 85%, about 90%, about 95%, about 96%, about 97%, about 98%, about 99% or about 100% homologous or identical to the sequence having a NCBI Reference No: NP_932170 (SEQ ID No: 32) or a fragment thereof, and/or may optionally comprise or consists of up to one or up to two or up to three conservative amino acid substitutions. In certain embodiments, the CD3ζ polypeptide comprises or consists of an amino acid sequence that is a consecutive portion of SEQ ID NO: 32, which is at least 20, or at least 30, or at least 40, or at least 50, and up to 164 amino acids in length. In certain embodiments, the CD3ζ polypeptide comprises or consists of an amino acid sequence of amino acids 1 to 164, 1 to 50, 50 to 100, 100 to 150, 52 or 164, or 150 to 164 of SEQ ID NO: 32. In certain embodiments, the intracellular signaling domain of the CAR comprises a CD3ζ polypeptide consisting of amino acids 52 to 164 of SEQ ID NO: 32. SEQ ID NO: 32 is provided below:

| [SEQ ID NO: 32] | |
|---|---|
| 1 | MKWKALFTAA ILQAQLPITE AQSFGLLDPK LCYLLDGILF IYGVILTALF LRVKFSRSAD |
| 61 | APAYQQGQNQ LYNELNLGRR EEYDVLDKRR GRDPEMGGKP QRRKNPQEGL YNELQKDKMA |
| 121 | EAYSEIGMKG ERRRGKGHDG LYQGLSTATK DTYDALHMQA LPPR |

In certain embodiments, the intracellular signaling domain of the CAR comprises a murine CD3ζ polypeptide. In certain embodiments, the CD3ζ polypeptide comprises or consists of an amino acid sequence that is at least about 85%, about 90%, about 95%, about 96%, about 97%, about 98%, about 99% or about 100% homologous or identical to the sequence having a NCBI Reference No: NP_001106864.2 (SEQ ID No: 33) or a fragment thereof, and/or may optionally comprise or consist of up to one or up to two or up to three conservative amino acid substitutions. In certain embodiments, the CD3ζ polypeptide comprises or consists of an amino acid sequence that is a consecutive portion of SEQ ID NO: 33, which is at least about 20, or at least about 30, or at least about 40, or at least about 50, or at least about 90, or at least about 100, and up to 188 amino acids in length. In certain embodiments, the CD3ζ polypeptide comprises or consists of an amino acid sequence of amino acids 1 to 164, 1 to 50, 50 to 100, 52 to 142, 100 to 150, or 150 to 188 of SEQ ID NO: 33. SEQ ID NO: 33 is provided below:

| [SEQ ID NO: 33] | |
|---|---|
| 1 | MKWKVSVLAC ILHVRFPGAE AQSFGLLDPK LCYLLDGILF IYGVIITALY LRAKFSRSAE |
| 61 | TAANLQDPNQ LYNELNLGRR EEYDVLEKKR ARDPEMGGKQ RRRNPQEGVY NALQKDKMAE |
| 121 | AYSEIGTKGE RRRGKGHDGL YQDSHFQAVQ FGNRREREGS ELTRTLGLRA RPKACRHKKP |
| 181 | LSLPAAVS |

In certain embodiments, the intracellular signaling domain of the CAR comprises a CD3ζ polypeptide comprising or consisting of the amino acid sequence set forth in SEQ ID NO: 34. SEQ ID NO: 34 is provided below.

[SEQ ID NO: 34]
RVKFSRSAEPPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

In certain embodiments, the intracellular signaling domain of the CAR comprises a modified CD3ζ polypeptide. In certain embodiments, the intracellular signaling domain of the CAR comprises a modified human CD3ζ polypeptide. In certain embodiments, the modified CD3ζ polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to SEQ ID NO: 35 or a fragment thereof, and/or may optionally comprise or consist of up to one or up to two or up to three conservative amino acid substitutions. SEQ ID NO: 35 is provided below:

[SEQ ID NO: 35]
RVKFSRSADA PAYQQGQNQL YNELNLGRRE EYDVLDKRRG
RDPEMGGKPR RKNPQEGLFN ELQKDKMAEA FSEIGMKGER
RRGKGHDGLF QGLSTATKDT FDALHMQALP PR

An exemplary nucleic acid sequence encoding the amino acid sequence of SEQ ID NO: 35 is set forth in SEQ ID NO: 36, which is provided below.

[SEQ ID NO: 36]
agagtgaagttcagcaggagcgcagacgcccccgcgtaccagcagggcca
gaaccagctctataacgagctcaatctaggacgaagagaggagtacgatg
ttttggacaagagacgtggccgggaccctgagatggggggaaagccgaga
aggaagaaccctcaggaaggcctgtTcaatgaactgcagaaagataagat
ggcggaggcctTcagtgagattgggatgaaaggcgagcgccggaggggca
aggggcacgatggcctttTccaggggctcagtacagccaccaaggacacc
tTcgacgcccttcacatgcaggccctgccccctcgc

In certain embodiments, the intracellular signaling domain of the CAR comprises a modified CD3ζ polypeptide comprising one, two or three ITAMs. In certain embodiments, the modified CD3ζ polypeptide comprises a native ITAM1 comprising or consisting of the amino acid sequence set forth in SEQ ID NO: 37.

[SEQ ID NO: 37]
QNQLYNELNLGRREEYDVLDKR

An exemplary nucleic acid sequence encoding the amino acid sequence of SEQ ID NO: 37 is set forth in SEQ ID NO: 38, which is provided below.

[SEQ ID NO: 38]
cagaaccagctctataacgagctcaatctagga
cgaagagaggagtacgatgttttggacaagaga

In certain embodiments, the modified CD3ζ polypeptide comprises an ITAM1 variant comprising one or more loss-of-function mutations. In certain embodiments, the ITAM1 variant comprises or consists of two loss-of-function mutations. In certain embodiments, each of the one or more (e.g., two) loss of function mutations comprises a mutation of a tyrosine residue in ITAM1. In certain embodiments, the ITAM1 variant (e.g., the variant consisting of two loss-of-function mutations) comprises or consists of the amino acid sequence set forth in SEQ ID NO: 39, which is provided below.

[SEQ ID NO: 39]
QNQLFNELNLGRREEFDVLDKR

An exemplary nucleic acid sequence encoding the amino acid sequence of SEQ ID NO: 39 is set forth in SEQ ID NO: 40, which is provided below.

[SEQ ID NO: 40]
cagaaccagctctTtaacgagctcaatctagga
cgaagagaggagtTcgatgttttggacaagaga

In certain embodiments, the modified CD3ζ polypeptide comprises a native ITAM2 comprising or consisting of the amino acid sequence set forth in SEQ ID NO: 41, which is provided below.

[SEQ ID NO: 41]
QEGLYNELQKDKMAEAYSEIGMK

An exemplary nucleic acid sequence encoding the amino acid sequence of SEQ ID NO: 41 is set forth in SEQ ID NO: 42, which is provided below.

[SEQ ID NO: 42]
caggaaggcctgtacaatgaactgcagaaagataagatggcggaggccta
cagtgagattgggatgaaa

In certain embodiments, the modified CD3ζ polypeptide comprises an ITAM2 variant comprising one or more loss-of-function mutations. In certain embodiments, the ITAM2 variant comprises or consists of two loss-of-function mutations. In certain embodiments, each of the one or more (e.g., two) the loss of function mutations comprises or consists of a mutation of a tyrosine residue in ITAM2. In certain embodiments, the ITAM2 variant (e.g., a variant consisting of two loss-of-function mutations) comprises or consists of the amino acid sequence set forth in SEQ ID NO: 43, which is provided below.

[SEQ ID NO: 43]
QEGLFNELQKDKMAEAFSEIGMK

An exemplary nucleic acid sequence encoding the amino acid sequence of SEQ ID NO: 43 is set forth in SEQ ID NO: 44, which is provided below.

[SEQ ID NO: 44]
caggaaggcctgtTcaatgaactgcagaaagataagatggcggaggcctT
cagtgagattgggatgaaa

In certain embodiments, the modified CD3ζ polypeptide comprises a native ITAM3 comprising or consisting of the amino acid sequence set forth in SEQ ID NO: 45, which is provided below.

[SEQ ID NO: 45]
HDGLYQGLSTATKDTYDALHMQ

An exemplary nucleic acid sequence encoding the amino acid sequence of SEQ ID NO: 45 is set forth in SEQ ID NO: 46, which is provided below.

[SEQ ID NO: 46]
cacgatggcctttaccagggtctcagtacagccaccaaggacacctacga
cgcccttcacatgcag

In certain embodiments, the modified CD3ζ polypeptide comprises an ITAM3 variant comprising one or more loss-of-function mutations. In certain embodiments, the ITAM3 variant comprises or consists of two loss-of-function mutations. In certain embodiments, each of the one or more (e.g., two) the loss of function mutations comprises or consists of a mutation of a tyrosine residue in ITAM3. In certain embodiments, the ITAM3 variant (e.g., a variant consisting of two loss-of-function mutations) comprises or consists of the amino acid sequence set forth in SEQ ID NO: 47, which is provided below. HDGLFQGLSTATKDTFDALHMQ [SEQ ID NO: 47]

An exemplary nucleic acid sequence encoding the amino acid sequence of SEQ ID NO: 47 is set forth in SEQ ID NO: 48, which is provided below.

[SEQ ID NO: 48]
cacgatggcctttTccaggggctcagtacagccaccaaggacacctTcga
cgcccttcacatgcag

In certain embodiments, the intracellular signaling domain of the CAR comprises a modified CD3ζ polypeptide comprising or consisting of a native ITAM1, an ITAM2 variant comprising or consisting of one or more loss-of-function mutations, and an ITAM3 variant comprising or consisting of one or more loss-of-function mutations, or a combination thereof. In certain embodiments, the ITAM2 variant comprises or consists of two loss-of-function mutations and the ITAM3 variant comprises or consists of two loss-of-function mutations. In certain embodiments, the intracellular signaling domain of the CAR comprises a modified CD3ζ polypeptide comprising or consisting of a native ITAM1, an ITAM2 variant comprising or consisting of two loss-of-function mutations and an ITAM3 variant comprising or consisting two loss-of-function mutations. In certain embodiments, the intracellular signaling domain of the CAR comprises a modified CD3ζ polypeptide comprising or consisting of a native ITAM1 consisting of the amino acid sequence set forth in SEQ ID NO: 37, an ITAM2 variant consisting of the amino acid sequence set forth in SEQ ID NO: 43 and an ITAM3 variant consisting of the amino acid sequence set forth in SEQ ID NO: 47. In certain embodiments, the CAR binds to CD19 and the CAR is designated as “1XX”. In certain embodiments, the modified CD3ζ polypeptide comprises or consists of the amino acid sequence set forth in SEQ ID NO: 35.

In certain embodiments, the intracellular signaling domain of the CAR comprises a modified CD3ζ polypeptide as disclosed in WO2019/133969, which is incorporated herein by reference.

In certain embodiments, the intracellular signaling domain of the CAR does not comprise a co-stimulatory signaling region, i.e., the CAR is a first-generation CAR.

In certain embodiments, the intracellular signaling domain of the CAR further comprises at least a co-stimulatory signaling region. In certain embodiments, the co-stimulatory signaling region comprises or consists of at least one co-stimulatory molecule or a portion thereof, which can provide optimal lymphocyte activation. As used herein, “co-stimulatory molecules” refer to cell surface molecules other than antigen receptors or their ligands that are required for an efficient response of lymphocytes to antigen. Co-stimulatory molecules can provide optimal lymphocyte activation. In certain embodiments, the at least one co-stimulatory signaling region comprises or consists of a CD28 polypeptide, a 4-1BB polypeptide, an OX40 polypeptide, an ICOS polypeptide, a DAP-10 polypeptide, or a combination thereof. In certain embodiments, the at least one co-stimulatory signaling region comprises or consists of a CD28 polypeptide. The co-stimulatory molecule can bind to a co-stimulatory ligand, which is a protein expressed on cell surface that upon binding to its receptor produces a co-stimulatory response, i.e., an intracellular response that effects the stimulation provided when an antigen binds to its CAR molecule. Co-stimulatory ligands, include, but are not limited to CD80, CD86, CD70, OX40L, and 4-1BBL. As one example, a 4-1BB ligand (i.e., 4-1BBL) may bind to 4-1BB (also known as “CD137”) for providing an intracellular signal that in combination with a CAR signal induces an effector cell function of the CAR⁺ T cell. CARs comprising an intracellular signaling domain that comprises a co-stimulatory signaling region comprising 4-1BB, ICOS or DAP-10 are disclosed in U.S. Pat. No. 7,446,190, which is herein incorporated by reference in its entirety.

In certain embodiments, the intracellular signaling domain of the CAR comprises a co-stimulatory signaling region that comprises or consists of a CD28 polypeptide. In certain embodiments, the intracellular signaling domain of the CAR comprises a co-stimulatory signaling region that comprises or consists of an intracellular domain of CD28 or a portion thereof. In certain embodiments, the co-stimulatory signaling region comprises or consists of an intracellular domain of human CD28 or a portion thereof. In certain embodiments, the CD28 polypeptide comprises or consists of an amino acid sequence that is at least about 85%, about 90%, about 95%, about 96%, about 97%, about 98%, about 99% or 100% homologous or identical to the amino acid sequence set forth in SEQ ID NO: 29, or fragments thereof, and/or may optionally comprise or consist of up to one or up to two or up to three conservative amino acid substitutions. In certain embodiments, the CD28 polypeptide comprises or consists of an amino acid sequence that is a consecutive portion of SEQ ID NO: 29 which is at least 20, or at least 30, or at least 40, or at least 50, and up to 220 amino acids in length. In certain embodiments, the CD28 polypeptide comprises or consists of an amino acid sequence of amino acids 1 to 220, 1 to 50, 50 to 100, 100 to 150, 114 to 220, 150 to 200, 181 to 220, or 200 to 220 of SEQ ID NO: 29. In certain embodiments, the CD28 polypeptide comprises or consists of an amino acid sequence of amino acids 181 to 220 of SEQ ID NO: 29.

In certain embodiments, the a co-stimulatory signaling region comprises or consists of an intracellular domain of mouse CD28 or a portion thereof. In certain embodiments, the CD28 polypeptide comprises or consists of an amino acid sequence that is at least about 85%, about 90%, about 95%, about 96%, about 97%, about 98%, about 99% or about 100% homologous or identical to the amino acid sequence set forth in SEQ ID NO: 31), or fragments thereof, and/or may optionally comprise or consist of up to one or up to two or up to three conservative amino acid substitutions. In certain embodiments, the CD28 polypeptide comprises or consists of an amino acid sequence that is a consecutive portion of SEQ ID NO: 31 which is at least about 20, or at least about 30, or at least about 40, or at least about 50, and up to 218 amino acids in length. In certain embodiments, the CD28 polypeptide comprises or consists of an amino acid sequence of amino acids 1 to 218, 1 to 50, 50 to 100, 100 to 150, 114 to 218, 115 to 218, 150 to 200, 178 to 218, or 200 to 218 of SEQ ID NO: 31. In certain embodiments, the CD28 polypeptide comprises or consists of an amino acid sequence of amino acids 115 to 218 of SEQ ID NO: 31.

In accordance with the presently disclosed subject matter, a “CD28 nucleic acid molecule” refers to a polynucleotide encoding a CD28 polypeptide.

In certain embodiments, the intracellular signaling domain of the CAR comprises a co-stimulatory signaling region that comprises or consists of intracellular domains of two co-stimulatory molecules or portions thereof: an intracellular domain of CD28 or a portion thereof and an intracellular domain of 4-1BB or a portion thereof, or an intracellular domain of CD28 or a portion thereof and an intracellular domain of OX40 or a portion thereof.

In certain embodiments, the intracellular signaling domain of the CAR comprises a co-stimulatory signaling region that comprises or consists of a 4-1BB polypeptide. In certain embodiments, the intracellular signaling domain of the CAR comprises a co-stimulatory signaling region that comprises or consists of an intracellular domain of 4-1BB or a portion thereof. In certain embodiments, the co-stimulatory signaling region comprises or consists of an intracellular domain of human 4-1BB or a portion thereof. 4-1BB can act as a tumor necrosis factor (TNF) ligand and have stimulatory activity. In certain embodiments, the 4-1BB polypeptide comprises or consists of an amino acid sequence that is at least about 85%, about 90%, about 95%, about 96%, about 97%, about 98%, about 99% or about 100% homologous or identical to the sequence having a NCBI Reference No: NP_001552 (SEQ ID NO: 49) or a fragment thereof, and/or may optionally comprise or consist of up to one or up to two or up to three conservative amino acid substitutions. In certain embodiments, the 4-1BB polypeptide comprises or consists of an amino acid sequence that is a consecutive portion of SEQ ID NO: 49, which is at least about 20, or at least about 30, or at least about 40, or at least about 50, and up to 255 amino acids in length. In certain embodiments, the 4-1BB polypeptide comprises or consists of an amino acid sequence of amino acids 1 to 255, 1 to 50, 50 to 100, 100 to 150, 150 to 200, 214-255 or 200 to 255 of SEQ ID NO: 49. In certain embodiments, the 4-1BB polypeptide comprises or consists of an amino acid sequence of amino acids 214-255 of SEQ ID NO: 49. SEQ ID NO: 49 is provided below:

| [SEQ ID NO: 49] | |
|---|---|
| 1 | MGNSCYNIVA TLLLVLNFER TRSLQDPCSN CPAGTFCDNN RNQICSPCPP NSFSSAGGQR |
| 61 | TCDICRQCKG VFRTRKECSS TSNAECDCTP GFHCLGAGCS MCEQDCKQGQ ELTKKGCKDC |
| 121 | CFGTFNDQKR GICRPWTNCS LDGKSVLVNG TKERDVVCGP SPADLSPGAS SVTPPAPARE |
| 181 | PGHSPQIISF FLALTSTALL FLLFFLTLRF SVVKRGRKKL LYIFKQPFMR PVQTTQEEDG |
| 241 | CSCRFPEEEE GGCEL |

In accordance with the presently disclosed subject matter, a “4-1BB nucleic acid molecule” refers to a polynucleotide encoding a 4-1BB polypeptide.

In certain embodiments, the co-stimulatory signaling region comprises an intracellular signaling domain of mouse 4-1BB or a portion thereof.

In certain embodiments, the intracellular signaling domain of the CAR comprises a co-stimulatory signaling region that comprises or consists of an OX40 polypeptide. In certain embodiments, the co-stimulatory signaling region comprises or consists of an intracellular domain of OX40 or a portion thereof. In certain embodiments, the co-stimulatory signaling region comprises or consists of an intracellular domain of human OX40 or a portion thereof. In certain embodiments, the OX40 polypeptide comprises or consists of an amino acid sequence that is at least about 85%, about 90%, about 95%, about 96%, about 97%, about 98%, about 99% or about 100% homologous or identical to the sequence having a NCBI Reference No: NP_003318 (SEQ ID NO: 50), or a fragment thereof, and/or may optionally comprise or consist of up to one or up to two or up to three conservative amino acid substitutions. In certain embodiments, the OX40 polypeptide comprises or consists of an amino acid sequence that is a consecutive portion of SEQ ID NO: 50, which is at least about 20, or at least about 30, or at least about 40, or at least about 50, and up to 277 amino acids in length. In certain embodiments, the 4-1BB polypeptide comprises or consists of an amino acid sequence of amino acids 1 to 277, 1 to 50, 50 to 100, 100 to 150, 150 to 200, or 200 to 277 of SEQ ID NO: 50. SEQ ID NO: 50 is provided below:

| [SEQ ID NO: 50] | |
|---|---|
| 1 | MCVGARRLGR GPCAALLLLG LGLSTVTGLH CVGDTYPSND RCCHECRPGN GMVSRCSRSQ |
| 61 | NTVCRPCGPG FYNDVVSSKP CKPCTWCNLR SGSERKQLCT ATQDTVCRCR AGTQPLDSYK |
| 121 | PGVDCAPCPP GHFSPGDNQA CKPWTNCTLA GKHTLQPASN SSDAICEDRD PPATQPQETQ |
| 181 | GPPARPITVQ PTEAWPRTSQ GPSTRPVEVP GGRAVAAILG LGLVLGLLGP LAILLALYLL |
| 241 | RRDQRLPPDA HKPPGGGSFR TPIQEEQADA HSTLAKI |

In accordance with the presently disclosed subject matter, an “OX40 nucleic acid molecule” refers to a polynucleotide encoding an OX40 polypeptide.

In certain embodiments, the intracellular signaling domain of the CAR comprises a co-stimulatory signaling region that comprises or consists of an ICOS polypeptide. In certain embodiments, the co-stimulatory signaling region comprises or consists of an intracellular domain of ICOS or a portion thereof. In certain embodiments, the co-stimulatory signaling region comprises or consists of an intracellular domain of human ICOS or a portion thereof. In certain embodiments, the ICOS polypeptide comprises or consists of an amino acid sequence that is at least about 85%, about 90%, about 95%, about 96%, about 97%, about 98%, about 99% or about 100% homologous or identical to the sequence having a NCBI Reference No: NP_036224 (SEQ ID NO: 51) or a fragment thereof, and/or may optionally comprise or consist of up to one or up to two or up to three conservative amino acid substitutions. In certain embodiments, the ICOS polypeptide comprises or consists of an amino acid sequence that is a consecutive portion of SEQ ID NO: 51 which is at least about 20, or at least about 30, or at least about 40, or at least about 50, and up to 199 amino acids in length. In certain embodiments, the ICOS polypeptide comprises or consists of an amino acid sequence of amino acids 1 to 277, 1 to 50, 50 to 100, 100 to 150, or 150 to 199 of SEQ ID NO: 51. SEQ ID NO: 51 is provided below:

[SEQ ID NO: 51]

| | |
|---|---|
| 1 | MKSGLWYFFL FCLRIKVLTG EINGSANYEM FIFHNGGVQI |
| | LCKYPDIVQQ FKMQLLKGGQ |
| 61 | ILCDLTKTKG SGNTVSIKSL KFCHSQLSNN SVSFFLYNLD |
| | HSHANYYFCN LSIFDPPPFK |
| 121 | VTLTGGYLHI YESQLCCQLK FWLPIGCAAF VVVCILGCIL |
| | ICWLTKKKYS SSVHDPNGEY |
| 181 | MFMRAVNTAK KSRLTDVTL |

In accordance with the presently disclosed subject matter, an “ICOS nucleic acid molecule” refers to a polynucleotide encoding an ICOS polypeptide.

3.3.4. Exemplary CARs

In certain embodiments, a presently disclosed CAR comprises or consists of a) an extracellular antigen-binding domain that binds to a CD19 polypeptide (e.g., a human CD19 polypeptide), b) a transmembrane domain comprising or consisting of a CD28 polypeptide (e.g., a transmembrane domain of human CD28 or a portion thereof), and c) an intracellular signaling domain comprising or consisting of a CD3ζ polypeptide and a co-stimulatory signaling region comprising or consisting of a CD28 polypeptide (e.g., an intracellular domain of human CD28 or a portion thereof). In certain embodiments, the CAR is designated as “CD1928ζ”. In certain embodiments, the CAR (e.g., CD1928ζ) comprises or consists of the amino acid sequence set forth in SEQ ID NO: 52. SEQ ID NO: 52 is provided below.

[SEQ ID NO: 52]
ALPVTALLLPLALLLHAEVKLQQSGAELVRPGSSVKISCKASGYAFSSYW
MNWVKQRPGQGLEWIGQIYPGDGDTNYNGKFKGQATLTADKSSSTAYMQL
SGLTSEDSAVYFCARKTISSVVDFYFDYWGQGTTVTVSSGGGGSGGGGSG
GGGSDIELTQSPKFMSTSVGDRVSVTCKASQNVGTNVAWYQQKPGQSPKP
LIYSATYRNSGVPDRFTGSGSGTDFTLTITNVQSKDLADYFCQQYNRYPY
TSGGGTKLEIKRAAAIEVMYPPPYLDNEKSNGTIIHVKGKHLCPSPLFPG
PSKPFWVLVVVGGVLACYSLLVTVAFIIFWVRSKRSRLLHSDYMNMTPRR
PGPTRKHYQPYAPPRDFAAYRSRVKFSRSAEPPAYQQGQNQLYNELNLGR
REEYDVLDKRRGRDPEMGGKPRRKNPQEGLYNELQKDKMAEAYSEIGMKG
ERRRGKGHDGLYQGLSTATKDTYDALHMQALPPR

An exemplary nucleic acid sequence encoding the amino acid sequence of SEQ ID NO: 52 is set forth in SEQ ID NO: 53. SEQ ID NO: 53 is provided below.

[SEQ ID NO: 53]
gctctcccagtgactgccctactgcttcccctagcgcttctcctgcatgc
agaggtgaagctgcagcagtctggggctgagctggtgaggcctgggtcct
cagtgaagatttcctgcaaggcttctggctatgcattcagtagctactgg
atgaactgggtgaagcagaggcctggacagggtcttgagtggattggaca
gatttatcctggagatggtgatactaactacaatggaaagttcaagggtc
aagccacactgactgcagacaaatcctccagcacagcctacatgcagctc
agcggcctaacatctgaggactctgcggtctatttctgtgcaagaaagac
cattagttcggtagtagatttctactttgactactggggccaagggacca
cggtcaccgtctcctcaggtggaggtggatcaggtggaggtggatctggt
ggaggtggatctgacattgagctcacccagtctccaaaattcatgtccac
atcagtaggagacagggtcagcgtcacctgcaaggccagtcagaatgtgg
gtactaatgtagcctggtatcaacagaaaccaggacaatctcctaaacca
ctgatttactcggcaacctaccggaacagtggagtccctgatcgcttcac
aggcagtggatctgggacagatttcactctcaccatcactaacgtgcagt
ctaaagacttggcagactatttctgtcaacaatataacaggtatccgtac
acgtccggaggggggaccaagctggagatcaaacgggcggccgcaattga
agttatgtatcctcctccttacctagacaatgagaagagcaatggaacca
ttatccatgtgaaagggaaacacctttgtccaagtcccctatttcccgga
ccttctaagcccttttgggtgctggtggtggttggtggagtcctggcttg
ctatagcttgctagtaacagtggcctttattattttctgggtgaggagta
agaggagcaggctcctgcacagtgactacatgaacatgactccccgccgc
cccgggcccacccgcaagcattaccagccctatgccccaccacgcgactt
cgcagcctatcgctccagagtgaagttcagcaggagcgcagagccccccg
cgtaccagcagggccagaaccagctctataacgagctcaatctaggacga
agagaggagtacgatgttttggacaagagacgtggccgggaccctgagat
ggggggaaagccgagaaggaagaaccctcaggaaggcctgtacaatgaac
tgcagaaagataagatggcggaggcctacagtgagattgggatgaaaggc
gagcgccggaggggcaaggggcacgatggcctttaccagggtctcagtac
agccaccaaggacacctacgacgcccttcacatgcaggccctgccccctc
gc

In certain embodiments, the cell comprises i) a presently disclosed dominant negative Fas polypeptide and ii) a CAR comprising or consisting of a) an extracellular antigen-binding domain that binds to CD19 (e.g., human CD19), b) a transmembrane domain comprising or consisting of a CD28 polypeptide (e.g., human CD28 polypeptide, e.g., a transmembrane domain of CD28 (e.g., human CD28) or a portion thereof), and c) an intracellular signaling domain comprising or consisting of a modified CD3ζ polypeptide comprising or consisting of a native ITAM1 consisting of the amino acid sequence set forth in SEQ ID NO: 37, an ITAM2 variant consisting of the amino acid sequence set forth in SEQ ID NO: 43 and an ITAM3 variant consisting of the amino acid sequence set forth in SEQ ID NO: 47, and a co-stimulatory signaling region comprising or consisting of a CD28 polypeptide (e.g., human CD28 polypeptide, e.g., an intracellular domain of CD28 (e.g., human CD28) or a portion thereof). In certain embodiments, the CAR is designated as “1928ζXX”. In certain embodiments, the CAR (e.g., 1928ζXX) comprises or consists of an amino acid sequence that is at least about 85%, about 90%, about 95%, about 96%, about 97%, about 98%, about 99% or about 100% homologous or identical to the amino acid sequence set forth in SEQ ID NO: 54, which is provided below. SEQ ID NO: 54 is able to bind to CD19 (e.g., human CD19).

[SEQ ID NO: 54]
MALPVTALLLPLALLLHAEVKLQQSGAELVRPGSSVKISCKASGYAFSSY
WMNWVKQRPGQGLEWIGQIYPGDGDTNYNGKFKGQATLTADKSSSTAYMQ
LSGLTSEDSAVYFCARKTISSVVDFYFDYWGQGTTVTVSSGGGGSGGGGS
GGGGSDIELTQSPKFMSTSVGDRVSVTCKASQNVGTNVAWYQQKPGQSPK
PLIYSATYRNSGVPDRFTGSGSGTDFTLTITNVQSKDLADYFCQQYNRYP
YTSGGGTKLEIKRAAAIEVMYPPPYLDNEKSNGTIIHVKGKHLCPSPLFP
GPSKPFWVLVVVGGVLACYSLLVTVAFIIFWVRSKRSRLLHSDYMNMTPR
RPGPTRKHYQPYAPPRDFAAYRSRVKFSRSADAPAYQQGQNQLYNELNLG
RREEYDVLDKRRGRDPEMGGKPRRKNPQEGLFNELQKDKMAEAFSEIGMK
GERRRGKGHDGLFQGLSTATKDTFDALHMQALPPR

An exemplary nucleic acid sequence encoding the amino acid sequence of SEQ ID NO: 54 is set forth in SEQ ID NO: 55, which is provided below.

[SEQ ID NO: 55]
atggctctcccagtgactgccctactgcttcccctagcgcttctcctgca
tgcagaggtgaagctgcagcagtctggggctgagctggtgaggcctgggt
cctcagtgaagatttcctgcaaggcttctggctatgcattcagtagctac
tggatgaactgggtgaagcagaggcctggacagggtcttgagtggattgg
acagatttatcctggagatggtgatactaactacaatggaaagttcaagg
gtcaagccacactgactgcagacaaatcctccagcacagcctacatgcag
ctcagcggcctaacatctgaggactctgcggtctatttctgtgcaagaaa
gaccattagttcggtagtagatttctactttgactactggggccaaggga
ccacggtcaccgtctcctcaggtggaggtggatcaggtggaggtggatct
ggtggaggtggatctgacattgagctcacccagtctccaaaattcatgtc
cacatcagtaggagacagggtcagcgtcacctgcaaggccagtcagaatg
tgggtactaatgtagcctggtatcaacagaaaccaggacaatctcctaaa
ccactgatttactcggcaacctaccggaacagtggagtccctgatcgctt
cacaggcagtggatctgggacagatttcactctcaccatcactaacgtgc
agtctaaagacttggcagactatttctgtcaacaatataacaggtatccg
tacacgtccggaggggggaccaagctggagatcaaacgggcggccgcaat
tgaagttatgtatcctcctccttacctagacaatgagaagagcaatggaa
ccattatccatgtgaaagggaaacacctttgtccaagtcccctatttccc
ggaccttctaagcccttttgggtgctggtggtggttggtggagtcctggc
ttgctatagcttgctagtaacagtggcctttattattttctgggtgagga
gtaagaggagcaggctcctgcacagtgactacatgaacatgactccccgc
cgccccgggcccacccgcaagcattaccagccctatgccccaccacgcga
cttcgcagcctatcgctccagagtgaagttcagcaggagcgcagacgccc
ccgcgtaccagcagggccagaaccagctctataacgagctcaatctagga
cgaagagaggagtacgatgttttggacaagagacgtggccgggaccctga
gatggggggaaagccgagaaggaagaaccctcaggaaggcctgtTcaatg
aactgcagaaagataagatggcggaggcctTcagtgagattgggatgaaa
ggcgagcgccggaggggcaaggggcacgatggcctttTccaggggctcag
tacagccaccaaggacacctTcgacgcccttcacatgcaggccctgcccc
ctcgctaa

### 4. Cells

The presently disclosed subject matter provides cells comprising a dominant negative Fas polypeptide disclosed herein. In certain embodiments, the cell further comprises an antigen-recognizing receptor (e.g., a CAR or a TCR) that binds to an antigen. In certain embodiments, the dominant negative Fas polypeptide is an exogenous dominant negative Fas polypeptide. In certain embodiments, the antigen-recognizing receptor is capable of activating the cell. In certain embodiments, the dominant negative Fas polypeptide (e.g., an exogenous dominant negative Fas polypeptide) is capable of promoting an anti-tumor effect of the cell. The cells can be transduced with an antigen-recognizing receptor and an exogenous dominant negative Fas polypeptide such that the cells co-express the antigen-recognizing receptor and the exogenous dominant negative Fas polypeptide.

In certain embodiments, the cell is an immunoresponsive cell. In certain embodiments, the cell is a cell of the lymphoid lineage. Cells of the lymphoid lineage produce antibodies, regulate cellular immune system, and detect foreign agents in the blood and cells foreign to the host and the like. Non-limiting examples of cells of the lymphoid lineage include T cells, Natural Killer (NK) cells, B cells, dendritic cells, and stem cells from which lymphoid cells may be differentiated. In certain embodiments, the stem cell is a pluripotent stem cell (e.g., embryonic stem cell or induced pluripotent stem cell).

In certain embodiments, the cell is a T cell. T cells can be lymphocytes that mature in the thymus and are chiefly responsible for cell-mediated immunity. T cells are part of the adaptive immune system. In certain embodiments, the T cells provided herein comprise any type of T cells, including, but not limited to, helper T cells, cytotoxic T cells, memory T cells (including central memory T cells, stem-cell-like memory T cells (or stem-like memory T cells), and two types of effector memory T cells: e.g., T_{EM} cells and T_{EMRA} cells, regulatory T cells (also known as suppressor T cells or T_{regs}), tumor-infiltrating lymphocytes (TILs), natural killer T cells, mucosal associated invariant T cells, and γδ T cells. Cytotoxic T cells (CTLs or killer T cells) are a subset of T lymphocytes capable of inducing the death of infected somatic or tumor cells. A patient's own T cells (i.e., autologous T cells) may be genetically modified to target specific antigens through the introduction of an antigen-recognizing receptor, e.g., a CAR or a TCR. In certain embodiments, the cell is a T cell. The T cell can be a CD4⁺ T cell or a CD8⁺ T cell. In certain embodiments, the T cell is a CD4⁺ T cell. In certain embodiments, the T cell is a CD8⁺ T cell.

In certain embodiments, the cell is a virus-specific T cell. In certain embodiments, the virus-specific T cell comprises an endogenous TCR that recognizes a viral antigen. In certain embodiments, the cell is a tumor-specific T cell. In certain embodiments, the tumor-specific T cell comprises an endogenous TCR that recognizes a tumor antigen (TSA or TAA).

In certain embodiments, the cell is an NK cell. Natural killer (NK) cells can be lymphocytes that are part of cell-mediated immunity and act during the innate immune response. NK cells do not require prior activation in order to perform their cytotoxic effect on target cells.

Types of human lymphocytes of the presently disclosed subject matter include, without limitation, peripheral donor lymphocytes, e.g., those disclosed in Sadelain, M., et al. 2003 Nat Rev Cancer 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the α and β heterodimer), in Panelli, M. C., et al. 2000 J Immunol 164:495-504; Panelli, M. C., et al. 2000 J Immunol 164:4382-4392 (disclosing lymphocyte cultures derived from tumor infiltrating lymphocytes (TILs) in tumor biopsies), and in Dupont, J., et al. 2005 Cancer Res 65:5417-5427; Papanicolaou, G. A., et al. 2003 Blood 102:2498-2505 (disclosing selectively in vitro-expanded antigen-specific peripheral blood leukocytes employing artificial antigen-presenting cells (AAPCs) or pulsed dendritic cells). The immunoresponsive cells (e.g., T cells) can be autologous, non-autologous (e.g., allogeneic), or derived in vitro from engineered progenitor or stem cells.

In certain embodiments, the cell is a cell of the myeloid lineage. Non-limiting examples of cells of the myeloid lineage include monocytes, macrophages, basophils, neutrophils, eosinophils, mast cell, erythrocytes, megakaryocytes, thrombocytes, and stem cells from which myeloid cells may be differentiated. In certain embodiments, the stem cell is a pluripotent stem cell (e.g., embryonic stem cell or induced pluripotent stem cell).

The presently disclosed cells are capable of modulating the tumor microenvironment. Tumors have a microenvironment that is hostile to the host immune response involving a series of mechanisms by malignant cells to protect themselves from immune recognition and elimination. This hostile “tumor microenvironment” comprises a variety of immune suppressive factors including infiltrating regulatory CD4⁺ T cells (Tregs), myeloid derived suppressor cells (MDSCs), tumor associated macrophages (TAMs), immune suppressive cytokines including TGF-β, and expression of ligands targeted to immune suppressive receptors expressed by activated T cells (CTLA-4 and PD-1). These mechanisms of immune suppression play a role in the maintenance of tolerance and suppressing inappropriate immune responses, however within the tumor microenvironment these mechanisms prevent an effective anti-tumor immune response. Collectively these immune suppressive factors can induce either marked anergy or apoptosis of adoptively transferred CAR modified T cells upon encounter with targeted tumor cells.

In certain embodiments, the presently disclosed cells have increased cell persistence. In certain embodiments, the presently disclosed cells have decreased apoptosis and/or anergy.

### 5. Compositions and Vectors

The presently disclosed subject matter provides compositions comprising a dominant negative Fas polypeptide disclosed herein (e.g., disclosed in Section 2) and an antigen-recognizing receptor disclosed herein (e.g., disclosed in Section 3). Also provided are cells (e.g., immunoresponsive cells) comprising such compositions.

In certain embodiments, the dominant negative Fas polypeptide is operably linked to a first promoter. In certain embodiments, the antigen-recognizing receptor is operably linked to a second promoter.

Furthermore, the presently disclosed subject matter provides nucleic acid compositions comprising a first polynucleotide encoding a dominant negative Fas polypeptide disclosed herein (e.g., disclosed in Section 2) and a second polynucleotide encoding an antigen-recognizing receptor disclosed herein (e.g., disclosed in Section 3). Also provided are cells comprising such nucleic acid compositions.

In certain embodiments, the nucleic acid composition further comprises a first promoter that is operably linked to the dominant negative Fas polypeptide. In certain embodiments, the nucleic acid composition further comprises a second promoter that is operably linked to the antigen-recognizing receptor.

In certain embodiments, one or both of the first and second promoters are endogenous or exogenous. In certain embodiments, the exogenous promoter is selected from the group consisting of an elongation factor (EF)-1 promoter, a CMV promoter, a SV40 promoter, a PGK promoter, a long terminal repeat (LTR) promoter and a metallothionein promoter. In certain embodiments, one or both of the first and second promoters are inducible promoters. In certain embodiments, the inducible promoter is selected from the group consisting of a NFAT transcriptional response element (TRE) promoter, a CD69 promoter, a CD25 promoter, an IL-2 promoter, an IL-12 promoter, a p40 promoter, and a Bcl-xL promoter.

Furthermore, the presently disclosed subject matter provides vectors comprising the nucleic acid composition. In certain embodiments, the vector is a retroviral vector. In certain embodiments, the vector is a lentiviral vector.

The compositions and nucleic acid compositions can be administered to subjects or and/delivered into cells by methods known in the art or as described herein. Genetic modification of a cell (e.g., a T cell) can be accomplished by transducing a substantially homogeneous cell composition with a recombinant DNA construct. In certain embodiments, a retroviral vector (either a gamma-retroviral vector or a lentiviral vector) is employed for the introduction of the DNA construct into the cell. For example, a first polynucleotide encoding an antigen-recognizing receptor and the second polynucleotide encoding the dominant negative Fas polypeptide can be cloned into a retroviral vector and expression can be driven from its endogenous promoter, from the retroviral long terminal repeat, or from a promoter specific for a target cell type of interest. Non-viral vectors may be used as well.

For initial genetic modification of a cell to include a dominant negative Fas polypeptide and an antigen-recognizing receptor (e.g., a CAR or a TCR), a retroviral vector is generally employed for transduction, however any other suitable viral vector or non-viral delivery system can be used. The antigen-recognizing receptor and the dominant negative Fas polypeptide can be constructed in a single, multicistronic expression cassette, in multiple expression cassettes of a single vector, or in multiple vectors. Examples of elements that can be used to create a multicistronic expression cassette include, but is not limited to, various viral and non-viral Internal Ribosome Entry Sites (IRES, e.g., FGF-1 IRES, FGF-2 IRES, VEGF IRES, IGF-II IRES, NF-κB IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides, e.g., P2A, T2A, E2A and F2A peptides). Combinations of retroviral vector and an appropriate packaging line are also suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD114 or GALV envelope and any other known in the art.

Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

Other transducing viral vectors can be used to modify a cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; LeGal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for genetic modification of a cell. For example, a nucleic acid molecule can be introduced into an immunoresponsive cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by microinjection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g. Zinc finger nucleases, meganucleases, or TALE nucleases, CRISPR). Transient expression may be obtained by RNA electroporation.

Any targeted genome editing methods can also be used to deliver the dominant negative Fas polypeptide and/or the antigen-recognizing receptor disclosed herein to a cell or a subject. In certain embodiments, a CRISPR system is used to deliver the dominant negative Fas polypeptide and/or the antigen-recognizing receptor disclosed herein. In certain embodiments, zinc-finger nucleases are used to deliver the dominant negative Fas polypeptide and/or the antigen-recognizing receptor disclosed herein. In certain embodiments, a TALEN system is used to deliver the dominant negative Fas polypeptide and/or the antigen-recognizing receptor disclosed herein.

The clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. The crRNA needs to be designed for each application as this is the sequence that Cas9 uses to identify and directly bind to the target DNA in a cell. The repair template carrying CAR expression cassette need also be designed for each application, as it must overlap with the sequences on either side of the cut and code for the insertion sequence. Multiple crRNA's and the tracrRNA can be packaged together to form a single-guide RNA (sgRNA). This sgRNA can be joined together with the Cas9 gene and made into a plasmid in order to be transfected into cells.

A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of basepairs. The most common method to generate new zinc-finger domain is to combine smaller zinc-finger “modules” of known specificity. The most common cleavage domain in ZFNs is the non-specific cleavage domain from the type IIs restriction endonuclease FokI. Using the endogenous homologous recombination (HR) machinery and a homologous DNA template carrying CAR expression cassette, ZFNs can be used to insert the CAR expression cassette into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome.

Transcription activator-like effector nucleases (TALENs) are restriction enzymes that can be engineered to cut specific sequences of DNA. A TALENs system operates on almost the same principle as ZFNs. They are generated by combining a transcription activator-like effectors DNA-binding domain with a DNA cleavage domain. Transcription activator-like effectors (TALEs) comprise 33-34 amino acid repeating motifs with two variable positions that have a strong recognition for specific nucleotides. By assembling arrays of these TALEs, the TALE DNA-binding domain can be engineered to bind a desired DNA sequence, and thereby guide the nuclease to cut at specific locations in genomic DNA sequences.

Polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g. the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

The resulting cells can be grown under conditions similar to those for unmodified cells, whereby the modified cells can be expanded and used for a variety of purposes.

### 6. Polypeptides and Analogs

Also included in the presently disclosed subject matter are a CD19, CD28, 4-1BB, CD8, CD3ζ, and Fas polypeptides or fragments thereof that are modified in ways that enhance their anti-neoplastic activity when expressed in an immunoresponsive cell. The presently disclosed subject matter provides methods for optimizing an amino acid sequence or nucleic acid sequence by producing an alteration in the sequence. Such alterations may include certain mutations, deletions, insertions, or post-translational modifications. The presently disclosed subject matter further includes analogs of any naturally-occurring polypeptide disclosed herein (including, but not limited to, CD19, CD8, 4-1BB, CD28, CD3ζ, and Fas). Analogs can differ from a naturally-occurring polypeptide disclosed herein by amino acid sequence differences, by post-translational modifications, or by both. Analogs can exhibit at least about 85%, about 90%, about 91%, about 92%, about 93%, about 94%, about 95%, about 96%, about 97%, about 98%, about 99% or more homologous or identical to all or part of a naturally-occurring amino acid sequence of the presently disclosed subject matter. The length of sequence comparison is at least 5, 10, 15 or 20 amino acid residues, e.g., at least 25, 50, or 75 amino acid residues, or more than 100 amino acid residues. Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., β or γ amino acids.

In addition to full-length polypeptides, the presently disclosed subject matter also provides fragments of any one of the polypeptides or peptide domains disclosed herein. As used herein, the term “a fragment” means at least 5, 10, 13, or 15 amino acids. In certain embodiments, a fragment comprises at least 20 contiguous amino acids, at least 30 contiguous amino acids, or at least 50 contiguous amino acids. In certain embodiments, a fragment comprises at least 60 to 80, 100, 200, 300 or more contiguous amino acids. Fragments can be generated by methods known to those skilled in the art or may result from normal protein processing (e.g., removal of amino acids from the nascent polypeptide that are not required for biological activity or removal of amino acids by alternative mRNA splicing or alternative protein processing events).

Non-protein analogs have a chemical structure designed to mimic the functional activity of a protein disclosed herein (e.g., dominant negative Fas polypeptide). Such analogs may exceed the physiological activity of the original polypeptide. Methods of analog design are well known in the art, and synthesis of analogs can be carried out according to such methods by modifying the chemical structures such that the resultant analogs increase the anti-neoplastic activity of the original polypeptide when expressed in an immunoresponsive cell. These chemical modifications include, but are not limited to, substituting alternative R groups and varying the degree of saturation at specific carbon atoms of a reference polypeptide. In certain embodiments, the protein analogs are relatively resistant to in vivo degradation, resulting in a more prolonged therapeutic effect upon administration. Assays for measuring functional activity include, but are not limited to, those described in the Examples below.

### 7. Administration

The presently disclosed cells or compositions comprising thereof can be provided systemically or directly to a subject for inducing and/or enhancing an immune response to an antigen and/or treating and/or preventing a neoplasm and/or a pathogen infection. In certain embodiments, the presently disclosed cells or compositions comprising thereof are directly injected into an organ of interest (e.g., an organ affected by a neoplasm). Alternatively, the presently disclosed cells or compositions comprising thereof are provided indirectly to the organ of interest, for example, by administration into the circulatory system (e.g., the tumor vasculature). Expansion and differentiation agents can be provided prior to, during or after administration of the cells or compositions to increase production of T cells or NK cells in vitro or in vivo.

The presently disclosed cells can be administered in any physiologically acceptable vehicle, normally intravascularly, although they may also be introduced into bone or other convenient site where the cells may find an appropriate site for regeneration and differentiation (e.g., the thymus). Usually, at least about 1×10⁵ cells will be administered, eventually reaching about 1×10¹⁰ or more. The presently disclosed cells can comprise a purified population of cells. Those skilled in the art can readily determine the percentage of the presently disclosed cells in a population using various well-known methods, such as fluorescence activated cell sorting (FACS). Suitable ranges of purity in populations comprising the presently disclosed cells are about 50% to about 55%, about 5% to about 60%, and about 65% to about 70%. In certain embodiments, the purity is about 70% to about 75%, about 75% to about 80%, or about 80% to about 85%. In certain embodiments, the purity is about 85% to about 90%, about 90% to about 95%, and about 95% to about 100%. Dosages can be readily adjusted by those skilled in the art (e.g., a decrease in purity may require an increase in dosage). The cells can be introduced by injection, catheter, or the like.

The presently disclosed compositions can be pharmaceutical compositions comprising the presently disclosed cells or their progenitors and a pharmaceutically acceptable carrier. Administration can be autologous or heterologous. For example, cells or progenitors can be obtained from one subject, and administered to the same subject or a different, compatible subject. Peripheral blood derived cells or their progeny (e.g., in vivo, ex vivo or in vitro derived) can be administered via localized injection, including catheter administration, systemic injection, localized injection, intravenous injection, or parenteral administration. When administering a presently disclosed therapeutic composition, it can be formulated in a unit dosage injectable form (solution, suspension, emulsion).

### 8. Formulations

Compositions comprising the presently disclosed cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the genetically modified immunoresponsive cells in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with the genetically modified immunoresponsive cells or their progenitors.

The compositions can be isotonic, i.e., they can have the same osmotic pressure as blood and lacrimal fluid. The desired isotonicity of the compositions may be accomplished using sodium chloride, or other pharmaceutically acceptable agents such as dextrose, boric acid, sodium tartrate, propylene glycol or other inorganic or organic solutes. Sodium chloride can be particularly for buffers containing sodium ions.

Viscosity of the compositions, if desired, can be maintained at the selected level using a pharmaceutically acceptable thickening agent. For example, methylcellulose is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

The quantity of cells to be administered will vary for the subject being treated. In a one embodiment, between about 10⁴ and about 10¹⁰, between about 10⁵ and about 10⁹, or between about 10⁶ and about 10⁸ of the presently disclosed cells are administered to a human subject. More effective cells may be administered in even smaller numbers. In certain embodiments, at least about 1×10⁸, about 2×10⁸, about 3×10⁸, about 4×10⁸, or about 5×10⁸ of the presently disclosed cells are administered to a human subject. The precise determination of what would be considered an effective dose may be based on factors individual to each subject, including their size, age, sex, weight, and condition of the particular subject. Dosages can be readily ascertained by those skilled in the art from this disclosure and the knowledge in the art.

The skilled artisan can readily determine the amount of cells and optional additives, vehicles, and/or carrier in compositions and to be administered in methods. Typically, any additives (in addition to the active cell(s) and/or agent(s)) are present in an amount of 0.001 to 50% (weight) solution in phosphate buffered saline, and the active ingredient is present in the order of micrograms to milligrams, such as about 0.0001 to about 5 wt %, about 0.0001 to about 1 wt %, about 0.0001 to about 0.05 wt % or about 0.001 to about 20 wt %, about 0.01 to about 10 wt %, or about 0.05 to about 5 wt %. For any composition to be administered to an animal or human, the followings can be determined: toxicity such as by determining the lethal dose (LD) and LD50 in a suitable animal model e.g., rodent such as mouse; the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

### 9. Methods of Treatment

The presently disclosed subject matter provides methods for inducing and/or increasing an immune response in a subject in need thereof. The presently disclosed cells and compositions comprising thereof can be used for treating and/or preventing a neoplasm in a subject. The presently disclosed cells and compositions comprising thereof can be used for prolonging the survival of a subject suffering from a neoplasm. The presently disclosed cells and compositions comprising thereof can also be used for treating and/or preventing a neoplasm in a subject. The presently disclosed cells and compositions comprising thereof can also be used for reducing tumor burden in a subject. The presently disclosed cells and compositions comprising thereof can also be used for treating and/or preventing a pathogen infection or other infectious disease in a subject, such as an immunocompromised human subject. Such methods comprise administering the presently disclosed cells in an amount effective or a composition (e.g., pharmaceutical composition) comprising thereof to achieve the desired effect, be it palliation of an existing condition or prevention of recurrence. For treatment, the amount administered is an amount effective in producing the desired effect. An effective amount can be provided in one or a series of administrations. An effective amount can be provided in a bolus or by continuous perfusion.

For adoptive immunotherapy using antigen-specific T cells, cell doses in the range of about 10⁶-10¹¹ (e.g., about 10⁹) are typically infused. Upon administration of the presently disclosed cells into the host and subsequent differentiation, T cells are induced that are specifically directed against the specific antigen. The modified cells can be administered by any method known in the art including, but not limited to, intravenous, subcutaneous, intranodal, intratumoral, intrathecal, intrapleural, intraperitoneal, intra-medullary and directly to the thymus.

The presently disclosed subject matter provides methods for treating and/or preventing a neoplasm in a subject. In certain embodiments, the method comprises administering an effective amount of the presently disclosed cells or a composition comprising thereof to a subject having a neoplasm.

In certain embodiments, the neoplasm is a malignant neoplasm. In certain embodiments, the neoplasms or tumors are cancers that have increased FASLG RNA expression relative to matched normal tissues of origin. See Yamamoto et al., J Clin Invest. (2019); 129(4):1551-1565, which is incorporated by reference herein.

Non-limiting examples of neoplasms (e.g., malignant neoplasms) include blood cancers (e.g. leukemias, lymphomas, and myelomas), ovarian cancer, breast cancer, bladder cancer, brain cancer, colon cancer, intestinal cancer, liver cancer, lung cancer, pancreatic cancer, prostate cancer, skin cancer, stomach cancer, glioblastoma, throat cancer, melanoma, neuroblastoma, adenocarcinoma, glioma, soft tissue sarcoma, and various carcinomas (including prostate and small cell lung cancer). Suitable carcinomas further include any known in the field of oncology, including, but not limited to, astrocytoma, fibrosarcoma, myxosarcoma, liposarcoma, oligodendroglioma, ependymoma, medulloblastoma, primitive neural ectodermal tumor (PNET), chondrosarcoma, osteogenic sarcoma, pancreatic ductal adenocarcinoma, small and large cell lung adenocarcinomas, chordoma, angiosarcoma, endotheliosarcoma, squamous cell carcinoma, bronchoalveolarcarcinoma, epithelial adenocarcinoma, and liver metastases thereof, lymphangiosarcoma, lymphangioendotheliosarcoma, hepatoma, cholangiocarcinoma, synovioma, mesothelioma, Ewing's tumor, rhabdomyosarcoma, colon carcinoma, basal cell carcinoma, sweat gland carcinoma, papillary carcinoma, sebaceous gland carcinoma, papillary adenocarcinoma, cystadenocarcinoma, medullary carcinoma, bronchogenic carcinoma, renal cell carcinoma, bile duct carcinoma, choriocarcinoma, seminoma, embryonal carcinoma, Wilms' tumor, testicular tumor, medulloblastoma, craniopharyngioma, ependymoma, pinealoma, hemangioblastoma, acoustic neuroma, oligodendroglioma, meningioma, neuroblastoma, retinoblastoma, leukemia, multiple myeloma, Waldenstrom's macroglobulinemia, and heavy chain disease, breast tumors such as ductal and lobular adenocarcinoma, squamous and adenocarcinomas of the uterine cervix, uterine and ovarian epithelial carcinomas, prostatic adenocarcinomas, transitional squamous cell carcinoma of the bladder, B and T cell lymphomas (nodular and diffuse) plasmacytoma, acute and chronic leukemias, malignant melanoma, soft tissue sarcomas and leiomyosarcomas. In certain embodiments, the neoplasm (e.g., malignant neoplasm) is selected from the group consisting of blood cancers (e.g. leukemias, lymphomas, and myelomas), ovarian cancer, prostate cancer, breast cancer, bladder cancer, brain cancer, colon cancer, intestinal cancer, liver cancer, lung cancer, pancreatic cancer, prostate cancer, skin cancer, stomach cancer, glioblastoma, and throat cancer. In certain embodiments, the presently disclosed immunoresponsive cells and compositions comprising thereof can be used for treating and/or preventing blood cancers (e.g., leukemias, lymphomas, and myelomas) or ovarian cancer, which are not amenable to conventional therapeutic interventions.

In certain embodiments, the neoplasm is a solid cancer or a solid tumor. In certain embodiments, the solid tumor or solid cancer is selected from the group consisting of glioblastoma, prostate adenocarcinoma, kidney papillary cell carcinoma, sarcoma, ovarian cancer, pancreatic adenocarcinoma, rectum adenocarcinoma, colon adenocarcinoma, esophageal carcinoma, uterine corpus endometrioid carcinoma, breast cancer, skin cutaneous melanoma, lung adenocarcinoma, stomach adenocarcinoma, cervical and endocervical cancer, kidney clear cell carcinoma, testicular germ cell tumors, and aggressive B-cell lymphomas.

The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

Suitable human subjects for therapy typically comprise two treatment groups that can be distinguished by clinical criteria. Subjects with “advanced disease” or “high tumor burden” are those who bear a clinically measurable tumor. A clinically measurable tumor is one that can be detected on the basis of tumor mass (e.g., by palpation, CAT scan, sonogram, mammogram or X-ray; positive biochemical or histopathologic markers on their own are insufficient to identify this population). A pharmaceutical composition is administered to these subjects to elicit an anti-tumor response, with the objective of palliating their condition. Ideally, reduction in tumor mass occurs as a result, but any clinical improvement constitutes a benefit. Clinical improvement includes decreased risk or rate of progression or reduction in pathological consequences of the tumor.

A second group of suitable subjects is known in the art as the “adjuvant group.” These are individuals who have had a history of a neoplasm, but have been responsive to another mode of therapy. The prior therapy can have included, but is not restricted to, surgical resection, radiotherapy, and traditional chemotherapy. As a result, these individuals have no clinically measurable tumor. However, they are suspected of being at risk for progression of the disease, either near the original tumor site, or by metastases. This group can be further subdivided into high-risk and low-risk individuals. The subdivision is made on the basis of features observed before or after the initial treatment. These features are known in the clinical arts, and are suitably defined for each different neoplasm. Features typical of high-risk subgroups are those in which the tumor has invaded neighboring tissues, or who show involvement of lymph nodes.

Another group have a genetic predisposition to neoplasm but have not yet evidenced clinical signs of neoplasm. For instance, women testing positive for a genetic mutation associated with breast cancer, but still of childbearing age, can wish to receive one or more of the immunoresponsive cells described herein in treatment prophylactically to prevent the occurrence of neoplasm until it is suitable to perform preventive surgery.

As a consequence of surface expression of an antigen-recognizing receptor that binds to a tumor antigen and a dominant negative Fas polypeptide (e.g., an exogenous dominant negative Fas polypeptide) that enhances the anti-tumor effect of the cells comprising the antigen-recognizing receptor and the dominant negative Fas polypeptide, adoptively transferred T or NK cells are endowed with augmented and selective cytolytic activity at the tumor site. Furthermore, subsequent to their localization to tumor or viral infection and their proliferation, the T cells turn the tumor or viral infection site into a highly conductive environment for a wide range of immune cells involved in the physiological anti-tumor or antiviral response (tumor infiltrating lymphocytes, NK-, NKT-cells, dendritic cells, and macrophages).

Additionally, the presently disclosed subject matter provides methods for treating and/or preventing a pathogen infection (e.g., viral infection, bacterial infection, fungal infection, parasite infection, or protozoal infection) in a subject, e.g., in an immunocompromised subject. The method can comprise administering an effective amount of the presently disclosed cells or a composition comprising thereof to a subject having a pathogen infection. Exemplary viral infections susceptible to treatment include, but are not limited to, Cytomegalovirus (CMV), Epstein Barr Virus (EBV), Human Immunodeficiency Virus (HIV), and influenza virus infections.

Further modification can be introduced to the presently disclosed cells (e.g., T cells) to avert or minimize the risks of immunological complications (known as “malignant T-cell transformation”), e.g., graft versus-host disease (GvHD), or when healthy tissues express the same target antigens as the tumor cells, leading to outcomes similar to GvHD. A potential solution to this problem is engineering a suicide gene into the presently disclosed cells. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. In certain embodiments, the suicide gene is an EGFRt polypeptide. The EGFRt polypeptide can enable T cell elimination by administering anti-EGFR monoclonal antibody (e.g., cetuximab). EGFRt can be covalently joined to the upstream of the antigen-recognizing receptor. The suicide gene can be included within the vector comprising nucleic acids encoding a presently disclosed CAR. In this way, administration of a prodrug designed to activate the suicide gene (e.g., a prodrug (e.g., AP1903 that can activate iCasp-9) during malignant T-cell transformation (e.g., GVHD) triggers apoptosis in the suicide gene-activated receptor-expressing (e.g., CAR-expressing) T cells. The incorporation of a suicide gene into the presently disclosed antigen-recognizing receptor (e.g., CAR) gives an added level of safety with the ability to eliminate the majority of receptor-expressing (e.g., CAR-expressing) T cells within a very short time period. A presently disclosed cell (e.g., a T cell) incorporated with a suicide gene can be pre-emptively eliminated at a given timepoint post T cell infusion, or eradicated at the earliest signs of toxicity.

### 10. Kits

The presently disclosed subject matter provides kits for inducing and/or enhancing an immune response and/or treating and/or preventing a neoplasm or a pathogen infection in a subject. In certain embodiments, the kit comprises an effective amount of presently disclosed cells or a pharmaceutical composition comprising thereof. In certain embodiments, the kit comprises a sterile container; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. In certain embodiments, the kit includes a nucleic acid molecule encoding an antigen-recognizing receptor (e.g., a CAR or a TCR) directed toward an antigen of interest and a nucleic acid molecule encoding a dominant negative Fas polypeptide in expressible form, which may optionally be present on one or more vectors.

If desired, the cells and/or nucleic acid molecules are provided together with instructions for administering the cells or nucleic acid molecules to a subject having or at risk of developing a neoplasm or pathogen or immune disorder. The instructions generally include information about the use of the composition for the treatment and/or prevention of a neoplasm or a pathogen infection. In certain embodiments, the instructions include at least one of the following: description of the therapeutic agent; dosage schedule and administration for treatment or prevention of a neoplasm, pathogen infection, or immune disorder or symptoms thereof; precautions; warnings; indications; counter-indications; over-dosage information; adverse reactions; animal pharmacology; clinical studies; and/or references. The instructions may be printed directly on the container (when present), or as a label applied to the container, or as a separate sheet, pamphlet, card, or folder supplied in or with the container.

### EXAMPLES

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides disclosed herein, and, as such, may be considered in making and practicing the presently disclosed subject matter. Particularly useful techniques for particular embodiments will be discussed in the sections that follow.

The following examples are put forth so as to provide those of ordinary skill in the art with a complete disclosure and description of how to make and use the presently disclosed cells and compositions, and are not intended to limit the scope of what the inventors regard as their invention.

### Example 1—T Cells Engineered with N-Terminal Mutated Fas DNR

### Methods and Materials

Cell cultures. Jurkat76 and Platinum-GP retroviral packaging cells (Cell Biolabs) were cultured in RPMI supplied with 10% fetal bovine serum, 10 mM HEPES (Gibco) and 25 Unit/ml PenStrep (Gibco). Primary T cells were cultured in RPMI supplied with 10% heat-inactivated human serum, 25 mM HEPES (Gibco) and 50 Unit/ml PenStrep (Gibco). Single clone selection of Fas CRISPR edited Jurkat cells was done by seeding a 96-well plate with 100 μl cells per well at density of 0.5 cells per well. Cells were cultured for 3 weeks and Fas surface expression was measured by flow cytometry.

Isolation and expansion of human T cells. Buffy coats were acquired from healthy donors at New York Blood Center. Peripheral blood mononuclear cells (PBMC) were isolated by density gradient centrifugation using Lymphocyte Separation Medium (Corning). CD8+ T cells were isolated using EasySep Human CD8+ T cell Isolation Kit (Stemcell). CD8+ T cells were activated on 5 μg/ml anti-CD3 (Miltenyi Biotec) antibody-coated plate and 1 μg/ml soluble anti-CD28 (Miltenyi Biotec). For viral transduction, T cells were treated with 50 IU/ml of IL-2 (PeproTech) for 2 days prior to transduction.

Plasmid design and viral transduction. All plasmids for viral packaging were designed based on SFGγ retroviral vector. A feline endogenous retrovirus envelope RD114 was used for co-transfection with SFGγ vector in Platinum-GP cell. Lipofectamine 3000 (ThermoFisher) was used for Platinum-GP cell co-transfection. Jurkat cells and primary T cells were transduced with viral supernatant on Retronectin (Takara) coated plate. Briefly, plate was coated with 20 ug/ml Retronectin at 4° C. overnight then blocked by PBS with 2% FBS for 30 min at room temperature. Plate was washed with PBS and loaded with viral supernatant. Centrifugation was done at 2000 g, 32° C. for 2 hr. Supernatant was aspirated and cells were loaded into each well. Plate was centrifuged again at 1200 rpm, 32° C. for 5 min and incubated at 37° C. for 2 days.

CRISPR editing. Fas exon2 targeting single guide RNA (sgRNA) was synthesized by Synthego with 20 nt targeting sequence: GUGACUGACAUCAACUCCAA (SEQ ID NO: 66) (chemically modified). Briefly, 2 μl of 50 μM sgRNA were mixed with 1 μl of 20 μM recombinant Cas9 protein (Synthego) at room temperature for 10 min. 1 million Jurkat cells were resuspended in 17 μl Nucleofector Solution P3 (Lonza) then mixed with sgRNA/Cas9 complex. Electroporation was done using Lonza 4D nucleofector with AXP-1004 16-well strip (Lonza). Electroporation program was set up as follows: Cell type: T cell human stim, using code EC 115. Post CRISPR editing Fas expression was measured by flow cytometry on day 4 post electroporation.

Sanger sequencing and sequence analysis. CRISPR editing efficiency was confirmed by analyzing sequencing data from PCR amplicons using PCR primers: 5′-TCTATCATTCATGGTGCTGTTTC-3′ (SEQ ID NO: 67) and 5′-AGGGGAACCAAAAACTGTAAAA-3′ (SEQ ID NO: 68). KOD Hot Start Master Mix (EMD Millipore) was used for PCR product. PCR amplicon purification and sequencing service was done by Genwiz. CRISPR edited sequences were compared to wild type control sequences using ICE sequencing software (Synthego).

Flow cytometry and intracellular staining. Conjugated antibodies used for flowcytometry includes Brilliant Violet 421TM anti-human EGFR (AY13, Biolegend), PE/Cy5 anti-human CD95 Fas (DX2, Biolegend), APC/Cyanine7 anti-human CD95 Fas (DX2, Biolegend), PerCP/Cyanine5.5 anti-human TNF-α (Mab 11, Biolegend). For NY-ESO targeting TCR, PE anti-TCR V1313.1 (IMMU 222, Beckman Coulter) was used. For CAR staining, an Alexa Fluor 647 AffiniPure F(ab′)₂ Fragment Goat Anti-Mouse IgG, F(ab′) 2 antibody (Jackson ImmunoResearch) was used. For cell viability, LIVE/DEAD™ Fixable Aqua Dead Cell Stain Kit (ThermoFisher) was used. For intracellular staining, Cytofix/Cytoperm™ Fixation/Permeabilization Solution Kit (BD Biosciences) was used following commercial standard protocol.

FasL apoptosis assay. A form of soluble FasL oligomerized through a leucine zipper motif (FasL-LZ) was used at 100 ng/ml for all apoptosis assays. Cells were treated with FasL-LZ at designed time points at 37° C. Cells were washed and stained for surface antibodies. Cells were stained with CellEvent™ Caspase-3/7 Green Detection Reagent (ThermoFisher) in FACS buffer for 25 min at 37° C. and washed twice. Cells were then stained with APC Annexin V (Biolegend) in Annexin V Binding Buffer (Biolegend) for 25 min at room temperature. Cells were washed twice and resuspended in Annexin V Binding Buffer for flowcytometry.

Statistical analysis. All statistical analyses were performed using the Prism 7 (GraphPad) software. No statistical methods were used to predetermine sample sizes. All analysis was done on triplicated samples. Statistical comparisons between two groups were calculated by paired Student's t-tests for matched samples. P<0.05 is considered statically important.

### Results

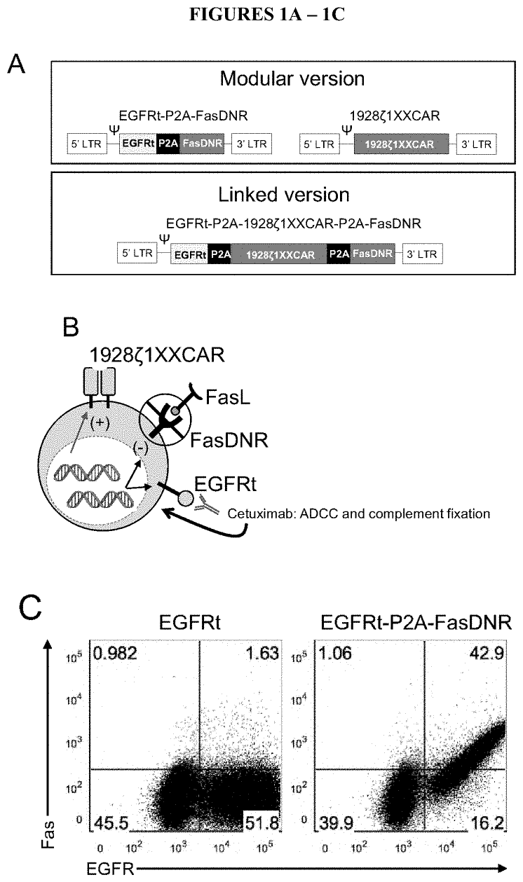

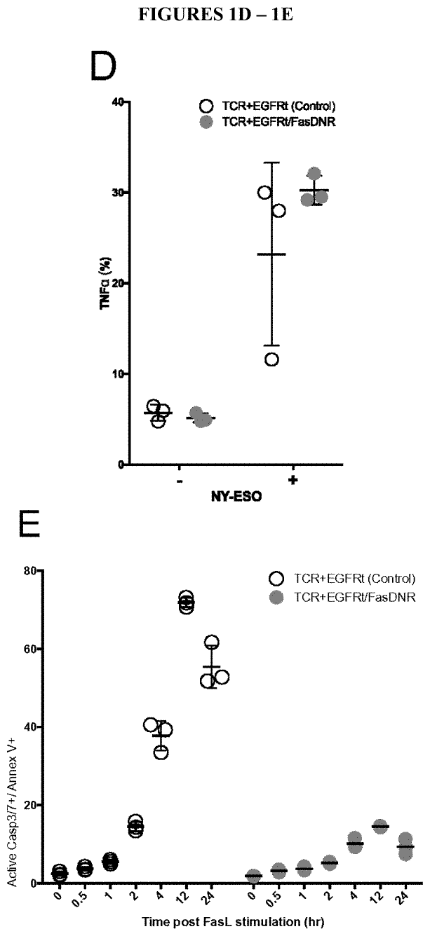

The functionality of T cells engineered with both a Fas DNR and a CAR was evaluated. Two versions of a human Fas DNR construct (see FIG. 1A) were generated: a modular version and a linked version. FIG. 1B demonstrates the activity of T cells comprising the FasDNR constructs. As shown in FIG. 1B, 192ζ1XXCAR targeted CD19⁺ malignant cells. FasDNR can protect T cells comprising the CAR and FasDNR from FasL-induced apoptosis. When administered with Cetuximab, EGFRt can be targeted, which induced antibody-dependent cell-mediated cytotoxicity (ADCC) or complement-dependent cytotoxicity.

Human-derived Jurkat cells were retrovirally transduced with EGFRt alone or EGFRt/FasDNR. Cells were stained on day 2 post transduction. As shown in FIG. 1C, Expression of FasDNR and EGFRt were proportional at ˜1:1 ratio in transduced cells.

Primary human CD8⁺ T cells were co-transduced with a TCR targeting NY-ESO antigen. Cells with or without FasDNR were exposed to antigen for 6 hours before intracellular cytokine staining. The results for intracellular staining of TNFα are shown in FIG. 1D. As show in FIG. 1D, antigen-specific TNFα expression in FasDNR transduced CD8⁺ T cells was not inferior to that of control cells not modified with the FasDNR. This finding indicates that the FasDNR does not reduce TNFα secretion in antigen activated T cells.

Primary human CD8⁺ T cells exposed to 100 ng/ml of FasL leucine zipper (FasL-1z) at designated time points. Activated Caspase3/7 and Annexin V were used as early apoptosis markers. The results are shown in FIG. 1E. As shown in FIG. 1E, FasL stimulation induced apoptosis in 70% of cells without FasDNR while <20% cells underwent apoptosis in FasDNR positive cells. This result confirms that FasDNR protects human CD8⁺ T cells from FasL induced apoptotic signaling.

In summary, FasDNR protected cells from FasL induced apoptosis and did not affect T cell tumor targeting functions.

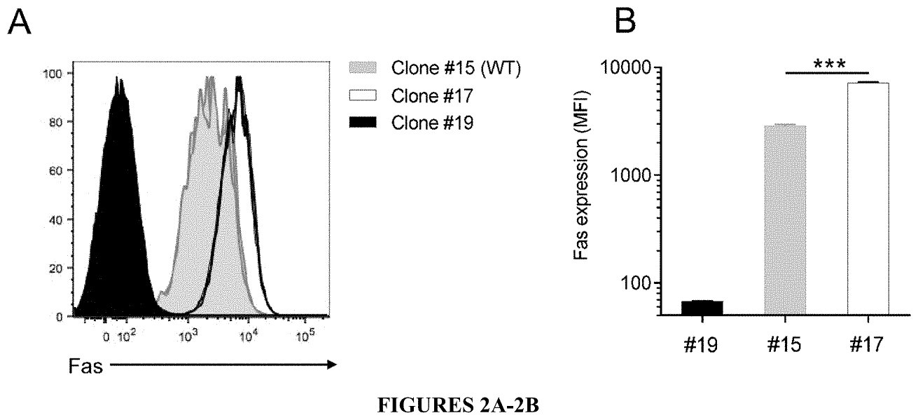

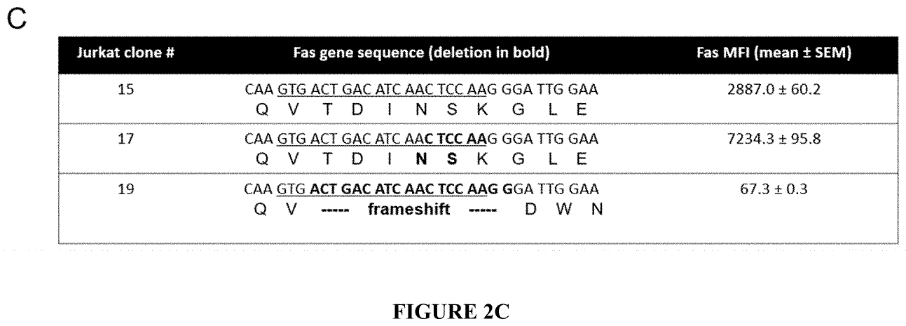

Using a CRISPR/Cas9 approach, a series of mutations were made in the N-terminal region of Fas and three main different T cell clones were identified: clone 15, having the wild type Fas exon 2 sequence (having the amino acid sequence set forth in SEQ ID NO: 10; clone 17, having a deletion of N31 and S32 in the N-terminal region as compared to the wild-type Fas (e.g., clone 15); and clone 19, having a bi-allelic 19 bases frameshift deletion. See FIG. 2C.

Jurkat cells electroporated with recombinant Cas9 protein loaded with synthetic guide (sg)RNA targeting the human Fas gene in exon 2. Fas surface expressions were measured on edited T cells following single cell cloning ˜3 weeks after electroporation. The results are shown in FIG. 2A. As shown in FIG. 2A, Clone #15 (gray) represented unedited wild type Fas cell surface expression. Clone #17 (clear) showed higher Fas expression level than wild type while clone #19 (black) showed minimal level of Fas. FIG. 2B shows mean Fas expression levels from FIG. 2A in triplicate samples. Clone #17 showed significantly higher Fas expression than clone #15.

In summary, clones 15, 17, and 19 showed different Fas expression levels, e.g., the Fas expression level of clone 17 was higher than that of clone 15 and clone 19.

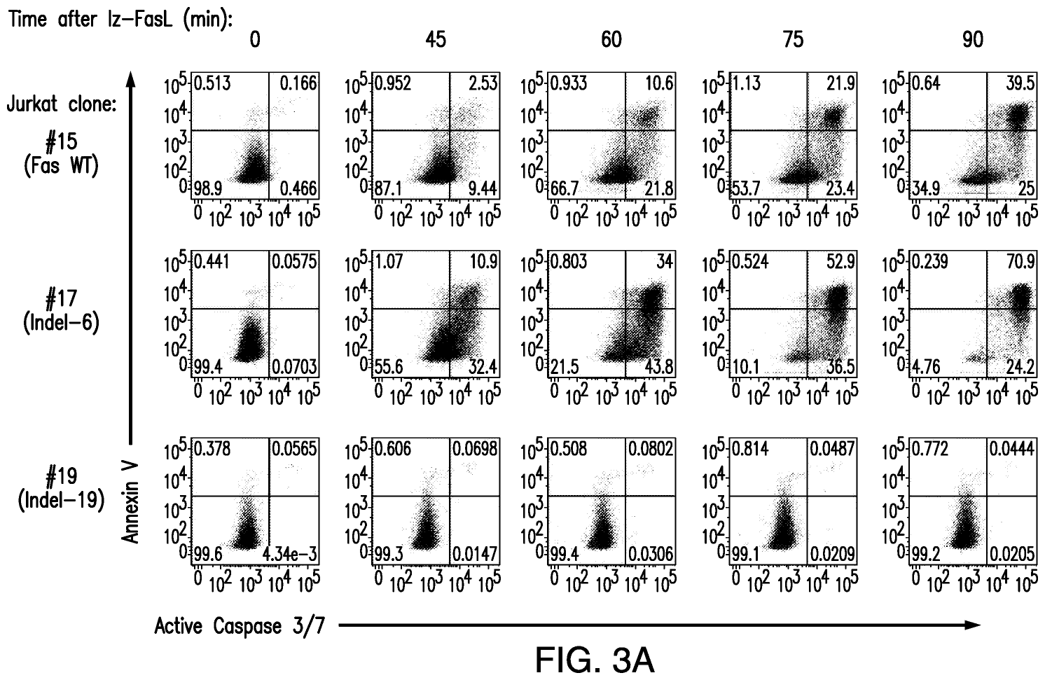

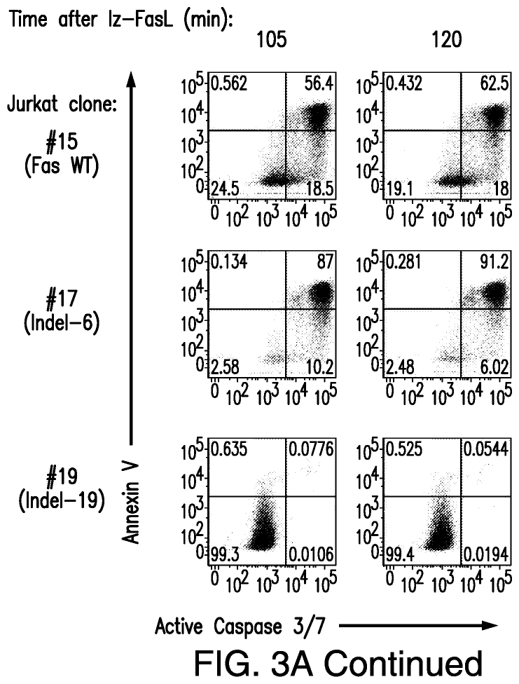

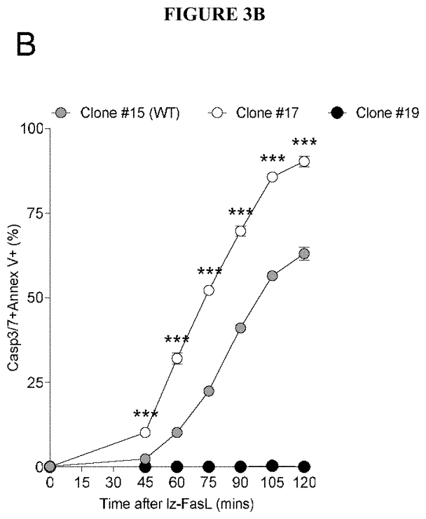

Next, the responsiveness of Jurket cells comprising clone 15, 17, or 19 to FasL stimulation was assessed. The results are shown in FIGS. 3A and 3B. As shown in FIGS. 3A and 3B, clone 17 showed sensitive response to FasL stimulation.

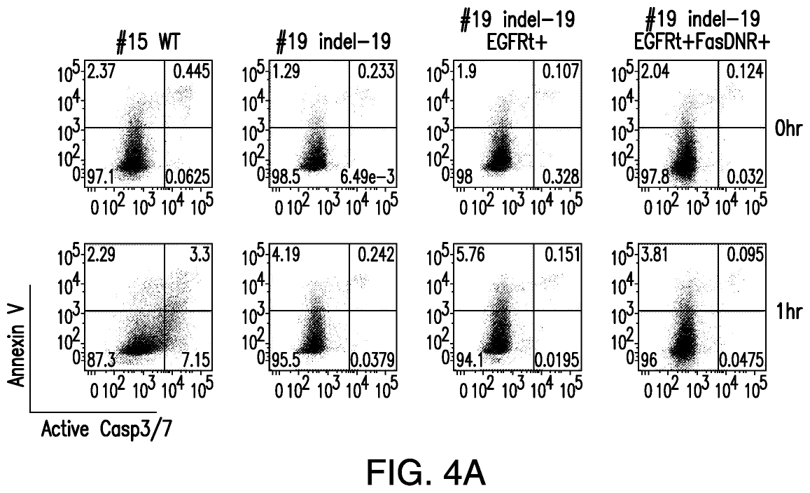

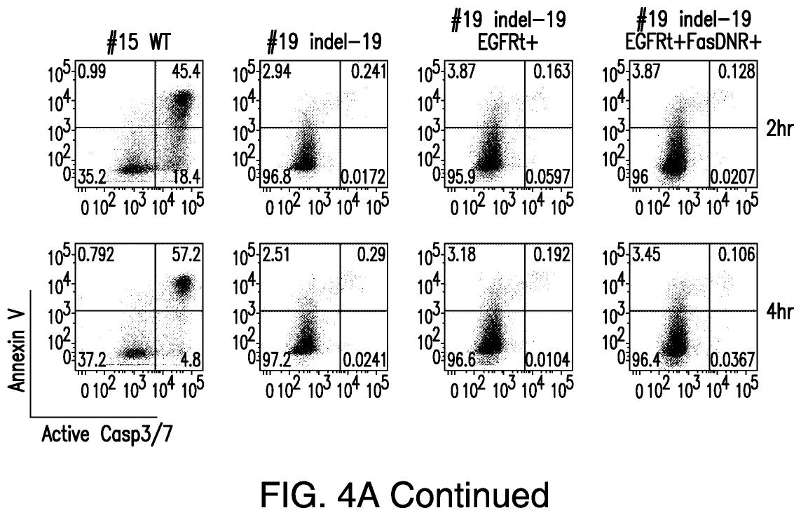

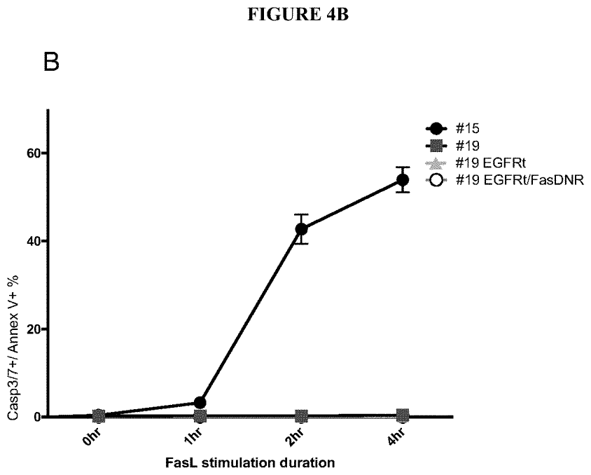

Jurkat cells were exposed to 100 ng/ml of FasL-LZ at designated time points. Apoptosis was assessed. The results are shown in FIGS. 4A and 4B. As shown in FIGS. 4A and 4B, Clone #15 with unedited wild type Fas underwent apoptosis following FasL stimulation. Fas knock-out clone #19 was resistant to apoptosis. In addition, FasDNR transduced clone #19 cells also protected from FasL induced apoptosis.

In summary, cells transduced with Fas knock-out clone #19 and FasDNR were protected from FasL induced apoptosis.

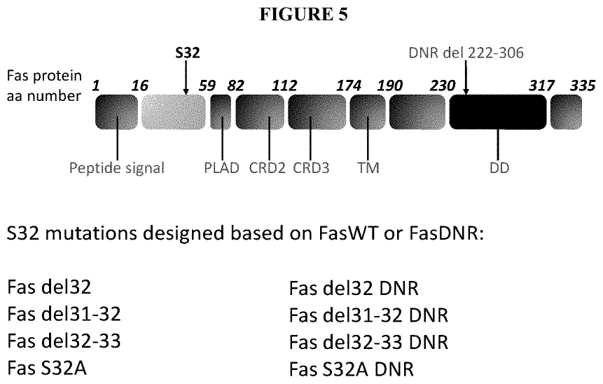

A number of N-terminal mutants were made, as shown in FIG. 5. Fas del32 DNR consists of a deletion of the amino acid at position 32 and a deletion of the amino acids at positions 230-314, and consists of the amino acid sequence set forth in SEQ ID NO: 16. Fas del31-32 DNR consists of a deletion of the amino acids at positions 31 and 32 and a deletion of the amino acids at positions 230-314, and consists of the amino acid sequence set forth in SEQ ID NO: 18. Fas del32-33 DNR consists of a deletion of the amino acids at positions 32-33 and a deletion of the amino acids at positions 230-314, and consists of the amino acid sequence set forth in SEQ ID NO: 20. Fas S32A DNR consists of a deletion of the amino acid at position 32 and a deletion of the amino acids at positions 230-314, and consists of the amino acid sequence set forth in SEQ ID NO: 24.

Fas del32 consists of the amino acid sequence set forth in SEQ ID NO: 56, which is provided below.

(SEQ ID NO: 56)
MLGIWTLLPLVLTSVARLSSKSVNAQVTDINKGLELRKTVTTVETQNLEG
LHHDGQFCHKPCPPGERKARDCTVNGDEPDCVPCQEGKEYTDKAHFSSKC
RRCRLCDEGHGLEVEINCTRTQNTKCRCKPNFFCNSTVCEHCDPCTKCEH
GIIKECTLTSNTKCKEEGSRSNLGWLCLLLLPIPLIVWVKRKEVQKTCRK
HRKENQGSHESPTLNPETVAINLSDVDLSKYITTIAGVMTLSQVKGFVRK
NGVNEAKIDEIKNDNVQDTAEQKVQLLRNWHQLHGKKEAYDTLIKDLKKA
NLCTLAEKIQTIILKDITSDSENSNFRNEIQSLV

An exemplary nucleotide sequence encoding the amino acid sequence of SEQ ID NO: 56 is set forth in SEQ ID NO: 57, which is provided below.

(SEQ ID NO: 57)
ATGCTGGGCATCTGGACCCTCCTACCTCTGGTTCTTACGTCTGTTGCTAG
ATTATCGTCCAAAAGTGTTAATGCCCAAGTGACTGACATCAACAAGGGAT
TGGAATTGAGGAAGACTGTTACTACAGTTGAGACTCAGAACTTGGAAGGC
CTGCATCATGATGGCCAATTCTGCCATAAGCCCTGTCCTCCAGGTGAAAG
GAAAGCTAGGGACTGCACAGTCAATGGGGATGAACCAGACTGCGTGCCCT
GCCAAGAAGGGAAGGAGTACACAGACAAAGCCCATTTTTCTTCCAAATGC
AGAAGATGTAGATTGTGTGATGAAGGACATGGCTTAGAAGTGGAAATAAA
CTGCACCCGGACCCAGAATACCAAGTGCAGATGTAAACCAAACTTTTTTT
GTAACTCTACTGTATGTGAACACTGTGACCCTTGCACCAAATGTGAACAT
GGAATCATCAAGGAATGCACACTCACCAGCAACACCAAGTGCAAAGAGGA
AGGATCCAGATCTAACTTGGGGTGGCTTTGTCTTCTTCTTTTGCCAATTC
CACTAATTGTTTGGGTGAAGAGAAAGGAAGTACAGAAAACATGCAGAAAG
CACAGAAAGGAAAACCAAGGTTCTCATGAATCTCCAACCTTAAATCCTGA
AACAGTGGCAATAAATTTATCTGATGTTGACTTGAGTAAATATATCACCA
CTATTGCTGGAGTCATGACACTAAGTCAAGTTAAAGGCTTTGTTCGAAAG
AATGGTGTCAATGAAGCCAAAATAGATGAGATCAAGAATGACAATGTCCA
AGACACAGCAGAACAGAAAGTTCAACTGCTTCGTAATTGGCATCAACTTC
ATGGAAAGAAAGAAGCGTATGACACATTGATTAAAGATCTCAAAAAAGCC
AATCTTTGTACTCTTGCAGAGAAAATTCAGACTATCATCCTCAAGGACAT
TACTAGTGACTCAGAAAATTCAAACTTCAGAAATGAAATCCAAAGCTTGG
TC

Fas del31-32 consists of the amino acid sequence set forth in SEQ ID NO: 58, which is provided below.

(SEQ ID NO: 58)
MLGIWTLLPLVLTSVARLSSKSVNAQVTDIKGLELRKTVTTVETQNLEGL
HHDGQFCHKPCPPGERKARDCTVNGDEPDCVPCQEGKEYTDKAHFSSKCR
RCRLCDEGHGLEVEINCTRTQNTKCRCKPNFFCNSTVCEHCDPCTKCEHG
IIKECTLTSNTKCKEEGSRSNLGWLCLLLLPIPLIVWVKRKEVQKTCRKH
RKENQGSHESPTLNPETVAINLSDVDLSKYITTIAGVMTLSQVKGFVRKN
GVNEAKIDEIKNDNVQDTAEQKVQLLRNWHQLHGKKEAYDTLIKDLKKAN
LCTLAEKIQTIILKDITSDSENSNFRNEIQSLV

An exemplary nucleotide sequence encoding the amino acid sequence of SEQ ID NO: 58 is set forth in SEQ ID NO: 59, which is provided below.

(SEQ ID NO: 59)
ATGCTGGGCATCTGGACCCTCCTACCTCTGGTTCTTACGTCTGTTGCTAG
ATTATCGTCCAAAAGTGTTAATGCCCAAGTGACTGACATCAAGGGATTGG
AATTGAGGAAGACTGTTACTACAGTTGAGACTCAGAACTTGGAAGGCCTG
CATCATGATGGCCAATTCTGCCATAAGCCCTGTCCTCCAGGTGAAAGGAA
AGCTAGGGACTGCACAGTCAATGGGGATGAACCAGACTGCGTGCCCTGCC
AAGAAGGGAAGGAGTACACAGACAAAGCCCATTTTTCTTCCAAATGCAGA
AGATGTAGATTGTGTGATGAAGGACATGGCTTAGAAGTGGAAATAAACTG
CACCCGGACCCAGAATACCAAGTGCAGATGTAAACCAAACTTTTTTTGTA
ACTCTACTGTATGTGAACACTGTGACCCTTGCACCAAATGTGAACATGGA
ATCATCAAGGAATGCACACTCACCAGCAACACCAAGTGCAAAGAGGAAGG
ATCCAGATCTAACTTGGGGTGGCTTTGTCTTCTTCTTTTGCCAATTCCAC
TAATTGTTTGGGTGAAGAGAAAGGAAGTACAGAAAACATGCAGAAAGCAC
AGAAAGGAAAACCAAGGTTCTCATGAATCTCCAACCTTAAATCCTGAAAC
AGTGGCAATAAATTTATCTGATGTTGACTTGAGTAAATATATCACCACTA
TTGCTGGAGTCATGACACTAAGTCAAGTTAAAGGCTTTGTTCGAAAGAAT
GGTGTCAATGAAGCCAAAATAGATGAGATCAAGAATGACAATGTCCAAGA
CACAGCAGAACAGAAAGTTCAACTGCTTCGTAATTGGCATCAACTTCATG
GAAAGAAAGAAGCGTATGACACATTGATTAAAGATCTCAAAAAAGCCAAT
CTTTGTACTCTTGCAGAGAAAATTCAGACTATCATCCTCAAGGACATTAC
TAGTGACTCAGAAAATTCAAACTTCAGAAATGAAATCCAAAGCTTGGTC

Fas del32-33 consists of the amino acid sequence set forth in SEQ ID NO: 60, which is provided below.

(SEQ ID NO: 60)
MLGIWTLLPLVLTSVARLSSKSVNAQVTDINGLELRKTVTTVETQNLEGL
HHDGQFCHKPCPPGERKARDCTVNGDEPDCVPCQEGKEYTDKAHFSSKCR
RCRLCDEGHGLEVEINCTRTQNTKCRCKPNFFCNSTVCEHCDPCTKCEHG
IIKECTLTSNTKCKEEGSRSNLGWLCLLLLPIPLIVWVKRKEVQKTCRKH
RKENQGSHESPTLNPETVAINLSDVDLSKYITTIAGVMTLSQVKGFVRKN
GVNEAKIDEIKNDNVQDTAEQKVQLLRNWHQLHGKKEAYDTLIKDLKKAN
LCTLAEKIQTIILKDITSDSENSNFRNEIQSLV

An exemplary nucleotide sequence encoding the amino acid sequence of SEQ ID NO: 60 is set forth in SEQ ID NO: 61, which is provided below.

(SEQ ID NO: 61)
ATGCTGGGCATCTGGACCCTCCTACCTCTGGTTCTTACGTCTGTTGCTAG
ATTATCGTCCAAAAGTGTTAATGCCCAAGTGACTGACATCAACGGATTGG
AATTGAGGAAGACTGTTACTACAGTTGAGACTCAGAACTTGGAAGGCCTG
CATCATGATGGCCAATTCTGCCATAAGCCCTGTCCTCCAGGTGAAAGGAA
AGCTAGGGACTGCACAGTCAATGGGGATGAACCAGACTGCGTGCCCTGCC
AAGAAGGGAAGGAGTACACAGACAAAGCCCATTTTTCTTCCAAATGCAGA
AGATGTAGATTGTGTGATGAAGGACATGGCTTAGAAGTGGAAATAAACTG
CACCCGGACCCAGAATACCAAGTGCAGATGTAAACCAAACTTTTTTTGTA
ACTCTACTGTATGTGAACACTGTGACCCTTGCACCAAATGTGAACATGGA
ATCATCAAGGAATGCACACTCACCAGCAACACCAAGTGCAAAGAGGAAGG
ATCCAGATCTAACTTGGGGTGGCTTTGTCTTCTTCTTTTGCCAATTCCAC
TAATTGTTTGGGTGAAGAGAAAGGAAGTACAGAAAACATGCAGAAAGCAC
AGAAAGGAAAACCAAGGTTCTCATGAATCTCCAACCTTAAATCCTGAAAC
AGTGGCAATAAATTTATCTGATGTTGACTTGAGTAAATATATCACCACTA
TTGCTGGAGTCATGACACTAAGTCAAGTTAAAGGCTTTGTTCGAAAGAAT
GGTGTCAATGAAGCCAAAATAGATGAGATCAAGAATGACAATGTCCAAGA
CACAGCAGAACAGAAAGTTCAACTGCTTCGTAATTGGCATCAACTTCATG
GAAAGAAAGAAGCGTATGACACATTGATTAAAGATCTCAAAAAAGCCAAT
CTTTGTACTCTTGCAGAGAAAATTCAGACTATCATCCTCAAGGACATTAC
TAGTGACTCAGAAAATTCAAACTTCAGAAATGAAATCCAAAGCTTGGTC

Fas S32A consists of the amino acid sequence set forth in SEQ ID NO: 62, which is provided below.

(SEQ ID NO: 62)
MLGIWTLLPLVLTSVARLSSKSVNAQVTDINAKGLELRKTVTTVETQNLE
GLHHDGQFCHKPCPPGERKARDCTVNGDEPDCVPCQEGKEYTDKAHFSSK
CRRCRLCDEGHGLEVEINCTRTQNTKCRCKPNFFCNSTVCEHCDPCTKCE
HGIIKECTLTSNTKCKEEGSRSNLGWLCLLLLPIPLIVWVKRKEVQKTCR
KHRKENQGSHESPTLNPETVAINLSDVDLSKYITTIAGVMTLSQVKGFVR
KNGVNEAKIDEIKNDNVQDTAEQKVQLLRNWHQLHGKKEAYDTLIKDLKK
ANLCTLAEKIQTIILKDITSDSENSNFRNEIQSLV

An exemplary nucleotide sequence encoding the amino acid sequence of SEQ ID NO: 62 is set forth in SEQ ID NO: 63, which is provided below.

(SEQ ID NO: 63)
ATGCTGGGCATCTGGACCCTCCTACCTCTGGTTCTTACGTCTGTTGCTAG
ATTATCGTCCAAAAGTGTTAATGCCCAAGTGACTGACATCAACGCCAAGG
GATTGGAATTGAGGAAGACTGTTACTACAGTTGAGACTCAGAACTTGGAA
GGCCTGCATCATGATGGCCAATTCTGCCATAAGCCCTGTCCTCCAGGTGA
AAGGAAAGCTAGGGACTGCACAGTCAATGGGGATGAACCAGACTGCGTGC
CCTGCCAAGAAGGGAAGGAGTACACAGACAAAGCCCATTTTTCTTCCAAA
TGCAGAAGATGTAGATTGTGTGATGAAGGACATGGCTTAGAAGTGGAAAT
AAACTGCACCCGGACCCAGAATACCAAGTGCAGATGTAAACCAAACTTTT
TTTGTAACTCTACTGTATGTGAACACTGTGACCCTTGCACCAAATGTGAA
CATGGAATCATCAAGGAATGCACACTCACCAGCAACACCAAGTGCAAAGA
GGAAGGATCCAGATCTAACTTGGGGTGGCTTTGTCTTCTTCTTTTGCCAA
TTCCACTAATTGTTTGGGTGAAGAGAAAGGAAGTACAGAAAACATGCAGA
AAGCACAGAAAGGAAAACCAAGGTTCTCATGAATCTCCAACCTTAAATCC
TGAAACAGTGGCAATAAATTTATCTGATGTTGACTTGAGTAAATATATCA
CCACTATTGCTGGAGTCATGACACTAAGTCAAGTTAAAGGCTTTGTTCGA
AAGAATGGTGTCAATGAAGCCAAAATAGATGAGATCAAGAATGACAATGT
CCAAGACACAGCAGAACAGAAAGTTCAACTGCTTCGTAATTGGCATCAAC
TTCATGGAAAGAAAGAAGCGTATGACACATTGATTAAAGATCTCAAAAAA
GCCAATCTTTGTACTCTTGCAGAGAAAATTCAGACTATCATCCTCAAGGA
CATTACTAGTGACTCAGAAAATTCAAACTTCAGAAATGAAATCCAAAGCT
TGGTC

Fas del33-34 was also made (not shown in FIG. 5). Fas del33-34 consists of the amino acid sequence set forth in SEQ ID NO: 64, which is provided below.

(SEQ ID NO: 64)
MLGIWTLLPLVLTSVARLSSKSVNAQVTDINSLELRKTVTTVETQNLEGL
HHDGQFCHKPCPPGERKARDCTVNGDEPDCVPCQEGKEYTDKAHFSSKCR
RCRLCDEGHGLEVEINCTRTQNTKCRCKPNFFCNSTVCEHCDPCTKCEHG
IIKECTLTSNTKCKEEGSRSNLGWLCLLLLPIPLIVWVKRKEVQKTCRKH
RKENQGSHESPTLNPETVAINLSDVDLSKYITTIAGVMTLSQVKGFVRKN
GVNEAKIDEIKNDNVQDTAEQKVQLLRNWHQLHGKKEAYDTLIKDLKKAN
LCTLAEKIQTIILKDITSDSENSNFRNEIQSLV

An exemplary nucleotide sequence encoding the amino acid sequence of SEQ ID NO: 64 is set forth in SEQ ID NO: 65, which is provided below.

(SEQ ID NO: 65)
ATGCTGGGCATCTGGACCCTCCTACCTCTGGTTCTTACGTCTGTTGCTAG
ATTATCGTCCAAAAGTGTTAATGCCCAAGTGACTGACATCAACTCATTGG
AATTGAGGAAGACTGTTACTACAGTTGAGACTCAGAACTTGGAAGGCCTG
CATCATGATGGCCAATTCTGCCATAAGCCCTGTCCTCCAGGTGAAAGGAA
AGCTAGGGACTGCACAGTCAATGGGGATGAACCAGACTGCGTGCCCTGCC
AAGAAGGGAAGGAGTACACAGACAAAGCCCATTTTTCTTCCAAATGCAGA
AGATGTAGATTGTGTGATGAAGGACATGGCTTAGAAGTGGAAATAAACTG
CACCCGGACCCAGAATACCAAGTGCAGATGTAAACCAAACTTTTTTTGTA
ACTCTACTGTATGTGAACACTGTGACCCTTGCACCAAATGTGAACATGGA
ATCATCAAGGAATGCACACTCACCAGCAACACCAAGTGCAAAGAGGAAGG
ATCCAGATCTAACTTGGGGTGGCTTTGTCTTCTTCTTTTGCCAATTCCAC
TAATTGTTTGGGTGAAGAGAAAGGAAGTACAGAAAACATGCAGAAAGCAC
AGAAAGGAAAACCAAGGTTCTCATGAATCTCCAACCTTAAATCCTGAAAC
AGTGGCAATAAATTTATCTGATGTTGACTTGAGTAAATATATCACCACTA
TTGCTGGAGTCATGACACTAAGTCAAGTTAAAGGCTTTGTTCGAAAGAAT
GGTGTCAATGAAGCCAAAATAGATGAGATCAAGAATGACAATGTCCAAGA
CACAGCAGAACAGAAAGTTCAACTGCTTCGTAATTGGCATCAACTTCATG
GAAAGAAAGAAGCGTATGACACATTGATTAAAGATCTCAAAAAAGCCAAT
CTTTGTACTCTTGCAGAGAAAATTCAGACTATCATCCTCAAGGACATTAC
TAGTGACTCAGAAAATTCAAACTTCAGAAATGAAATCCAAAGCTTGGTC

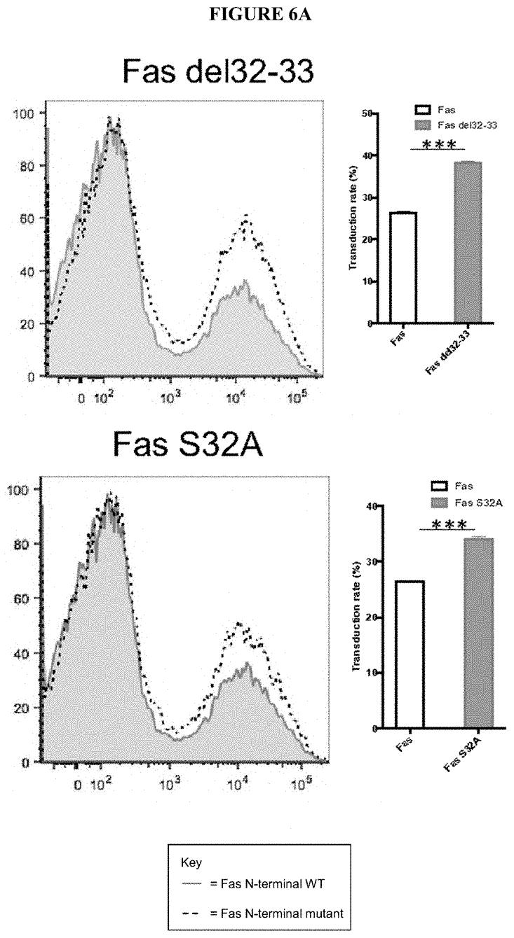

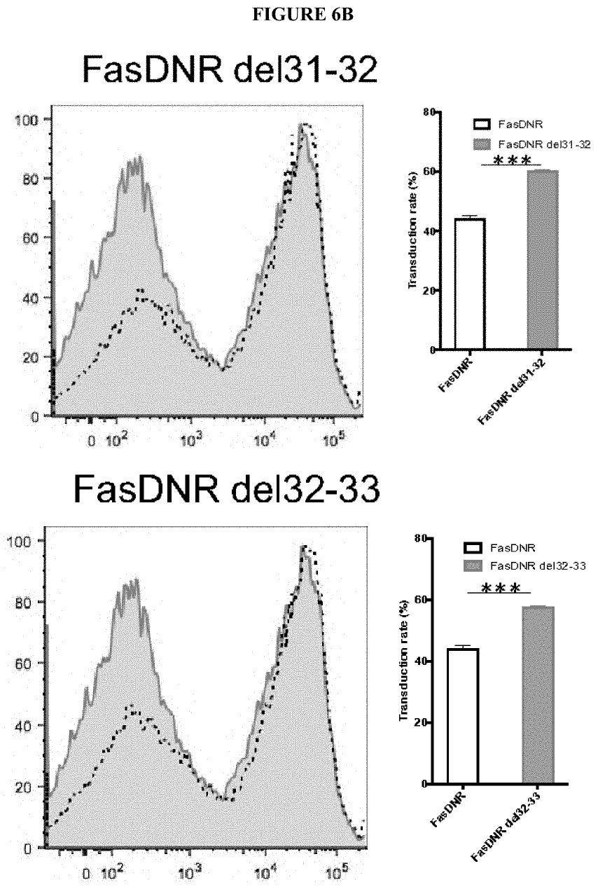

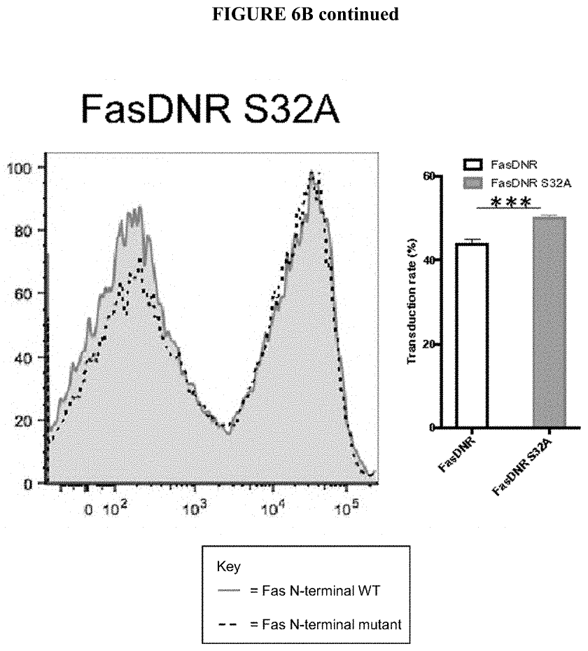

The transduction efficiency for Clone #19 Fas knock-out Jurkat cells with various Fas constructs was assessed. Clone #19 Fas knock-out Jurkat cells were transduced with Fas or FasDNR containing different N-terminal mutants and were stained for Fas expression on day 3 post trial transduction. The results are shown in FIGS. 6A and 6B. As shown in FIGS. 6A and 6B, Fas N-terminal mutants enhanced Fas transduction efficiency.

### Example 2—Sensitivity of Cells Expressing FasDNR with S32 or N31S32 Deletion to FasL Induced Apoptosis

### Methods and Materials

Human-derived Jurkat cells were retrovirally transduced with EGFRt/1928z with or without Fas mutations. Cells were isolated for EGFRt positive population on day 2 post transduction using Stemcell EasySep Human PE Positive Selection Kit II and PE anti-human EGFR antibody (Biolegend, clone AY13). On day 8 post transduction, FasL-LZ (100 ng/ml) was used for apoptosis assay at designed time points at 37° C. Cells were washed twice with FACS buffer and stained for surface antibodies. Cells were stained with CellEvent™ Caspase-3/7 Green Detection Reagent (ThermoFisher) in FACS buffer for 25 min at 37° C. and washed twice. Cells were then stained with APC Annexin V (Biolegend) in Annexin V Binding Buffer (Biolegend) for 25 min at room temperature. Cells were washed twice and resuspended in Annexin V Binding Buffer for flow cytometry.

### Results

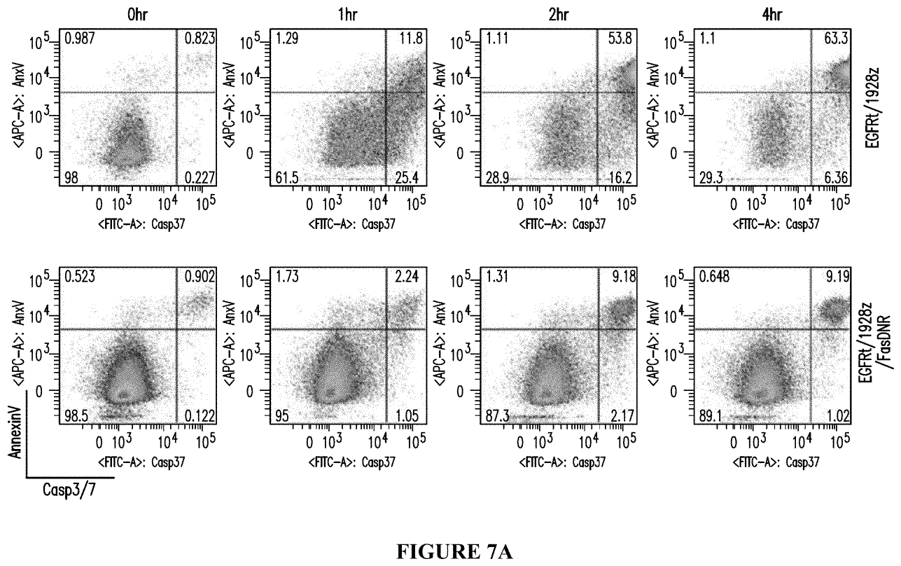

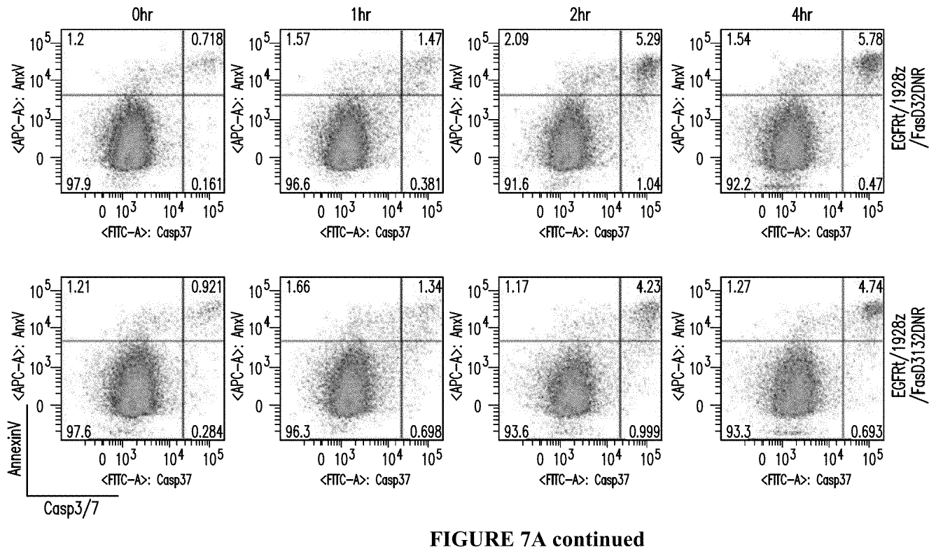

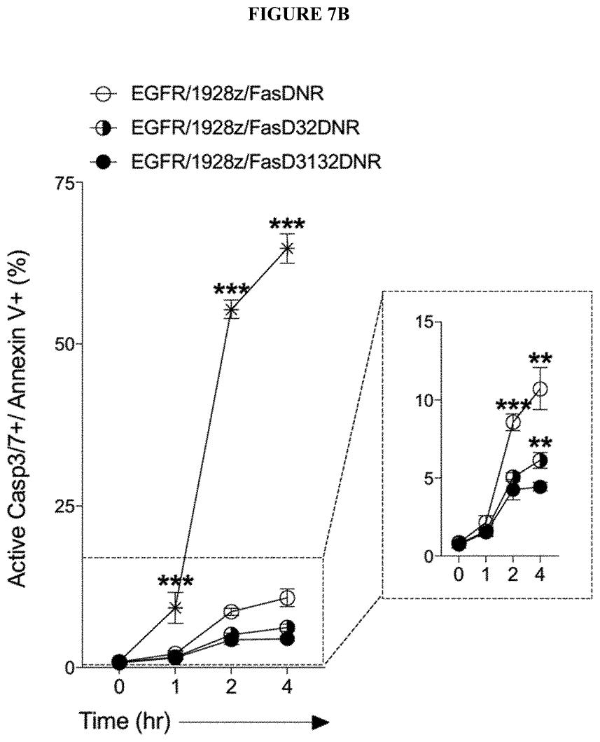

Jurkat cells were treated with FasL on day 8 post transduction. As shown in FIGS. 7A and 7B, FasD31DNR and FasD3132DNR expressing cells were better protected from FasL induced apoptosis (indicated by active Caspase3/7 and Annexin V double positive cell percentage) than cells expressing FasDNR without N-term mutations. p value was determined by unpaired Student's t-tests (** p<0.01, *** p<0.001).

### Example 3—Human Natural Killer Cells Engineered with N-Terminal Mutated Fas DNR

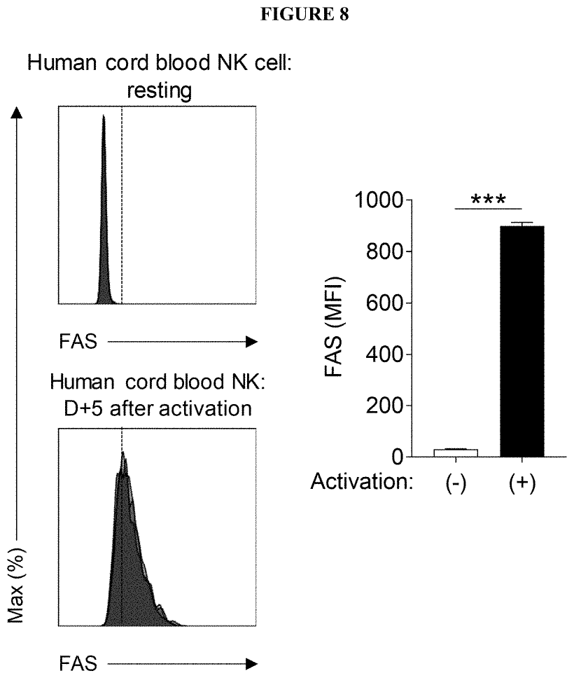

The utility and application of expressing N-terminal mutated Fas DNR in human NK cells was examined. Human NK cells were negatively isolated by magnetic beads from human cord blood. The NK cells were co-cultured with irradiated K562 (clone9) feeder cells at a 1:2 ratio (NK cells vs feeder cells). Media supplemented with 200 IU/ml of recombinant human IL-2 was added upon activation and was changed every other day. Fas expression was determined by flow cytometry on day 5 post activation. Upon activation, Fas expression was upregulated in human NK cells (see FIG. 8). Results of three independent cultures at rest and upon activation are shown as a bar graph+/−SEM. ***=P<0.001.

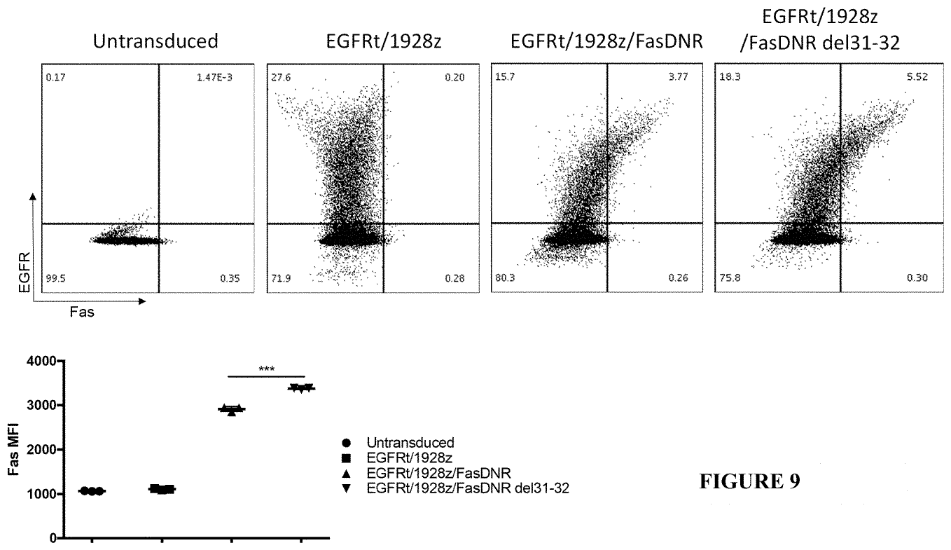

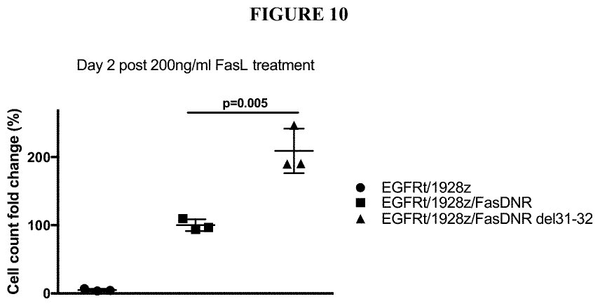

Fas expression levels in human NK cells transduced with a CAR, CAR-Fas DNR, or CAR-N-terminal mutant Fas DNR were examined. The N-terminal mutant Fas DNR (designed as “FasDNR del31-32” or “Fas del31-32 DNR”) consists of a deletion of the amino acids at positions 31 and 32 and a deletion of the amino acids at positions 230-314, and consists of the amino acid sequence set forth in SEQ ID NO: 18. Five-day activated human NK cells were transduced on 20 ug/ml Retronectin coated plate with DMEM media (untransduced control) or viral supernatant to produce NK cells transduced with EGFRt/1928z, EGFRt/1928z/FasDNR or EGFRt/1928z/FasDNR del31-32. Extra irradiated feeder cells were changed every other day. Cells were collected on day 4 post viral transduction for flow cytometry staining. ***=P<0.001. FIG. 9 demonstrated that human NK cells can be effectively transduced with Fas DNR and N-terminal mutant Fas DNR. FIG. 10 shows that N-terminal mutant Fas DNR modified human NK cells had significantly enhanced NK cell survival relative to Fas DNR and unmodified NK cells when exposed to recombinant FasL.

### REFERENCES

1. S. L. Maude et al., Tisagenlecleucel in Children and Young Adults with B-Cell Lymphoblastic Leukemia. N Engl J Med 378, 439-448 (2018).
2. S. S. Neelapu et al., Axicabtagene Ciloleucel CAR T-Cell Therapy in Refractory Large B-Cell Lymphoma. N Engl J Med 377, 2531-2544 (2017).
3. D. W. Lee et al., T cells expressing CD19 chimeric antigen receptors for acute lymphoblastic leukaemia in children and young adults: a phase 1 dose-escalation trial. Lancet 385, 517-528 (2015).
4. D. L. Porter et al., Chimeric antigen receptor T cells persist and induce sustained remissions in relapsed refractory chronic lymphocytic leukemia. Sci Transl Med 7, 303ra139 (2015).
5. C. J. Turtle et al., Immunotherapy of non-Hodgkin's lymphoma with a defined ratio of CD8+ and CD4+ CD19-specific chimeric antigen receptor-modified T cells. Sci Transl Med 8, 355ra116 (2016).
6. C. A. Ramos et al., Clinical and immunological responses after CD30-specific chimeric antigen receptor-redirected lymphocytes. J Clin Invest 127, 3462-3471 (2017).
7. J. H. Park et al., Long-Term Follow-up of CD19 CAR Therapy in Acute Lymphoblastic Leukemia. N Engl J Med 378, 449-459 (2018).
8. T. J. Fry et al., CD22-targeted CAR T cells induce remission in B-ALL that is naive or resistant to CD19-targeted CAR immunotherapy. Nat Med, (2017).
9. R. L. Siegel, K. D. Miller, A. Jemal, Cancer statistics, 2018. CA Cancer J Clin 68, 7-30 (2018).
10. C. U. Louis et al., Antitumor activity and long-term fate of chimeric antigen receptor-positive T cells in patients with neuroblastoma. Blood 118, 6050-6056 (2011).
11. M. R. Parkhurst et al., T cells targeting carcinoembryonic antigen can mediate regression of metastatic colorectal cancer but induce severe transient colitis. Mol Ther 19, 620-626 (2011).
12. N. Ahmed et al., Human Epidermal Growth Factor Receptor 2 (HER2)-Specific Chimeric Antigen Receptor-Modified T Cells for the Immunotherapy of HER2-Positive Sarcoma. J Clin Oncol 33, 1688-1696 (2015).
13. Y. C. Lu et al., Treatment of Patients With Metastatic Cancer Using a Major Histocompatibility Complex Class II-Restricted T-Cell Receptor Targeting the Cancer Germline Antigen MAGE-A3. J Clin Oncol 35, 3322-3329 (2017).
14. C. A. Klebanoff, S. A. Rosenberg, N. P. Restifo, Prospects for gene-engineered T cell immunotherapy for solid cancers. Nat Med 22, 26-36 (2016).
15. M. V. Maus, C. H. June, Making Better Chimeric Antigen Receptors for Adoptive T-cell Therapy. Clin Cancer Res 22, 1875-1884 (2016).
16. L. Gattinoni, C. A. Klebanoff, N. P. Restifo, Paths to stemness: building the ultimate antitumour T cell. Nat Rev Cancer 12, 671-684 (2012).
17. J. Zhu et al., Resistance to cancer immunotherapy mediated by apoptosis of tumor-infiltrating lymphocytes. Nat Commun 8, 1404 (2017).
18. S. A. Rosenberg et al., Durable complete responses in heavily pretreated patients with metastatic melanoma using T-cell transfer immunotherapy. Clin Cancer Res 17, 45504557 (2011).
19. S. Stevanovic et al., Landscape of immunogenic tumor antigens in successful immunotherapy of virally induced epithelial cancer. Science 356, 200-205 (2017).
20. A. Heczey et al., CAR T Cells Administered in Combination with Lymphodepletion and PD-1 Inhibition to Patients with Neuroblastoma. Mol Ther 25, 2214-2224 (2017).
21. E. A. Chong et al., PD-1 blockade modulates chimeric antigen receptor (CAR)-modified T cells: refueling the CAR. Blood 129, 1039-1041 (2017).
22. G. P. Adams et al., High affinity restricts the localization and tumor penetration of single-chain fv antibody molecules. Cancer Res 61, 4750-4755 (2001).
23. M. A. Postow, R. Sidlow, M. D. Hellmann, Immune-Related Adverse Events Associated with Immune Checkpoint Blockade. N Engl J Med 378, 158-168 (2018).
24. D. Aran et al., Comprehensive analysis of normal adjacent to tumor transcriptomes. Nat Commun 8, 1077 (2017).
25. G. T. Consortium, The Genotype-Tissue Expression (GTEx) project. Nat Genet 45, 580585 (2013).
26. B. Li, C. N. Dewey, RSEM: accurate transcript quantification from RNA-Seq data with or without a reference genome. BMC Bioinformatics 12, 323 (2011).
27. A. Subramanian et al., Gene set enrichment analysis: a knowledge-based approach for interpreting genome-wide expression profiles. Proc Natl Acad Sci USA 102, 1554515550 (2005).
28. D. Hamann et al., Phenotypic and functional separation of memory and effector human CD8+ T cells. J Exp Med 186, 1407-1418 (1997).
29. L. Gattinoni et al., A human memory T cell subset with stem cell-like properties. Nat Med 17, 1290-1297 (2011).
30. K. E. Pauken et al., Epigenetic stability of exhausted T cells limits durability of reinvigoration by PD-1 blockade. Science 354, 1160-1165 (2016).
31. C. L. Mackall et al., Distinctions between CD8+ and CD4+ T-cell regenerative pathways result in prolonged T-cell subset imbalance after intensive chemotherapy. Blood 89, 3700-3707 (1997).
32. D. Kagi et al., Fas and perforin pathways as major mechanisms of T cell-mediated cytotoxicity. Science 265, 528-530 (1994).
33. Z. Hao, T. W. Mak, Type I and type II pathways of Fas-mediated apoptosis are differentially controlled by XIAP. J Mol Cell Biol 2, 63-64 (2010).
34. A. S. Mohamood et al., Protection from autoimmune diabetes and T-cell lymphoproliferation induced by FasL mutation are differentially regulated and can be uncoupled pharmacologically. Am J Pathol 171, 97-106 (2007).
35. R. M. Siegel et al., Fas preassociation required for apoptosis signaling and dominant inhibition by pathogenic mutations. Science 288, 2354-2357 (2000).
36. M. P. Boldin et al., A novel protein that interacts with the death domain of Fas/APO1 contains a sequence motif related to the death domain. J Biol Chem 270, 7795-7798 (1995).
37. A. M. Chinnaiyan, K. O'Rourke, M. Tewari, V. M. Dixit, FADD, a novel death domain-containing protein, interacts with the death domain of Fas and initiates apoptosis. Cell 81, 505-512 (1995).
38. J. P. Medema et al., FLICE is activated by association with the CD95 death-inducing signaling complex (DISC). EMBO J 16, 2794-2804 (1997).
39. F. C. Kischkel et al., Cytotoxicity-dependent APO-1 (Fas/CD95)-associated proteins form a death-inducing signaling complex (DISC) with the receptor. EMBO J 14, 55795588 (1995).
40. C. H. June, B. R. Blazar, J. L. Riley, Engineering lymphocyte subsets: tools, trials and tribulations. Nat Rev Immunol 9, 704-716 (2009).
41. R. Watanabe-Fukunaga, C. I. Brannan, N. G. Copeland, N. A. Jenkins, S. Nagata, Lymphoproliferation disorder in mice explained by defects in Fas antigen that mediates apoptosis. Nature 356, 314-317 (1992).
42. M. Eberstadt, B. Huang, E. T. Olejniczak, S. W. Fesik, The lymphoproliferation mutation in Fas locally unfolds the Fas death domain. Nat Struct Biol 4, 983-985 (1997).
43. M. Ramaswamy et al., Specific elimination of effector memory CD4+ T cells due to enhanced Fas signaling complex formation and association with lipid raft microdomains. Cell Death Differ 18, 712-720 (2011).
44. J. R. Veatch et al., Tumor infiltrating BRAFV600E-specific CD4 T cells correlated with complete clinical response in melanoma. J Clin Invest, (2018).
45. S. S. Chandran et al., Treatment of metastatic uveal melanoma with adoptive transfer of tumour-infiltrating lymphocytes: a single-centre, two-stage, single-arm, phase 2 study. Lancet Oncol 18, 792-802 (2017).
46. A. G. Chapuis et al., T-Cell Therapy Using Interleukin-21-Primed Cytotoxic T-Cell Lymphocytes Combined With Cytotoxic T-Cell Lymphocyte Antigen-4 Blockade Results in Long-Term Cell Persistence and Durable Tumor Regression. J Clin Oncol 34, 37873795 (2016).
47. P. L. Cohen, R. A. Eisenberg, Lpr and gld: single gene models of systemic autoimmunity and lymphoproliferative disease. Annu Rev Immunol 9, 243-269 (1991).
48. V. K. Rao, J. B. Oliveira, How I treat autoimmune lymphoproliferative syndrome. Blood 118, 5741-5751 (2011).
49. H. C. Morse, 3rd et al., Abnormalities induced by the mutant gene Ipr: expansion of a unique lymphocyte subset. J Immunol 129, 2612-2615 (1982).
50. S. Izui et al., Induction of various autoantibodies by mutant gene lpr in several strains of mice. J Immunol 133, 227-233 (1984).
51. C. A. Klebanoff et al., Memory T cell-driven differentiation of naive cells impairs adoptive immunotherapy. J Clin Invest 126, 318-334 (2016).
52. A. C. Cruz et al., Fas/CD95 prevents autoimmunity independently of lipid raft localization and efficient apoptosis induction. Nat Commun 7, 13895 (2016).
53. C. A. Klebanoff et al., Central memory self/tumor-reactive CD8+ T cells confer superior antitumor immunity compared with effector memory T cells. Proc Natl Acad Sci USA 102, 9571-9576 (2005).
54. D. Sommermeyer et al., Chimeric antigen receptor-modified T cells derived from defined CD8+ and CD4+ subsets confer superior antitumor reactivity in vivo. Leukemia 30, 492500 (2016).
55. J. N. Kochenderfer et al., Lymphoma Remissions Caused by Anti-CD19 Chimeric Antigen Receptor T Cells Are Associated With High Serum Interleukin-15 Levels. J Clin Oncol 35, 1803-1813 (2017).
56. D. A. Martin et al., Defective CD95/APO-1/Fas signal complex formation in the human autoimmune lymphoproliferative syndrome, type Ia. Proc Natl Acad Sci US A 96, 45524557 (1999).
57. C. E. Jackson et al., Autoimmune lymphoproliferative syndrome with defective Fas: genotype influences penetrance. Am J Hum Genet 64, 1002-1014 (1999).
58. B. L. Horton, J. B. Williams, A. Cabanov, S. Spranger, T. F. Gajewski, Intratumoral CD8(+) T-cell Apoptosis Is a Major Component of T-cell Dysfunction and Impedes Antitumor Immunity. Cancer Immunol Res 6, 14-24 (2018).
59. M. A. Lakins, E. Ghorani, H. Munir, C. P. Martins, J. D. Shields, Cancer-associated fibroblasts induce antigen-specific deletion of CD8 (+) T Cells to protect tumour cells. Nat Commun 9, 948 (2018).
60. S. Kleber et al., Yes and PI3K bind CD95 to signal invasion of glioblastoma. Cancer Cell 13, 235-248 (2008).
61. M. E. Peter et al., The role of CD95 and CD95 ligand in cancer. Cell Death Differ 22, 549-559 (2015).
62. G. T. Motz et al., Tumor endothelium FasL establishes a selective immune barrier promoting tolerance in tumors. Nat Med 20, 607-615 (2014).
63. C. M. Bollard et al., Tumor-Specific T-Cells Engineered to Overcome Tumor Immune Evasion Induce Clinical Responses in Patients With Relapsed Hodgkin Lymphoma. J Clin Oncol, JCO2017743179 (2018).
64. L. Cherkassky et al., Human CART cells with cell-intrinsic PD-1 checkpoint blockade resist tumor-mediated inhibition. J Clin Invest 126, 3130-3144 (2016).
65. G. Dotti et al., Human cytotoxic T lymphocytes with reduced sensitivity to Fas-induced apoptosis. Blood 105, 4677-4684 (2005).
66. X. Wang et al., A transgene-encoded cell surface polypeptide for selection, in vivo tracking, and ablation of engineered cells. Blood 118, 1255-1263 (2011).
67. W. W. Overwijk et al., Tumor regression and autoimmunity after reversal of a functionally tolerant state of self-reactive CD8+ T cells. J Exp Med 198, 569-580 (2003).
68. S. P. Kerkar et al., Genetic engineering of murine CD8+ and CD4+ T cells for preclinical adoptive immunotherapy studies. J Immunother 34, 343-352 (2011).
69. P. F. Robbins et al., Tumor regression in patients with metastatic synovial cell sarcoma and melanoma using genetically engineered lymphocytes reactive with NY-ESO-1. J Clin Oncol 29, 917-924 (2011).
70. R. Eil et al., Ionic immune suppression within the tumour microenvironment limits T cell effector function. Nature 537, 539-543 (2016).
71. J. N. Kochendelfer et al., Adoptive transfer of syngeneic T cells transduced with a chimeric antigen receptor that recognizes murine CD19 can eradicate lymphoma and normal B cells. Blood. 2010; 116(19):3875-3886.
72. Y. Yang et al., TCR engagement negatively affect CD8 but not CD4 CAR T cell expansion and leukemic clearance. Sci Transl Med. 2017; 9(417):eaag1209.
73. H. Z. Imtiyaz et al., Structural requirements for signal-induced target binding of FADD determined by functional reconstitution of FADD deficiency. J Biol Chem. 2005; 280(36):31360-31367.
74. J. D. Mountz et al., Defective clonal deletion and anergy induction in TCR transgenic lpr/lpr mice. Semin Immunol. 1994; 6(1):27-37.
75. G. G. Singer et al., Apoptosis, Fas and systemic autoimmunity: the MRL-lpr/lpr model. Curre Opin Immunol. 1994; 6(6):913-920.
76. C. Y. Slaney et al., Dual-specific chimeric antigen receptor T cells and an indirect vaccine eradicate a variety of large solid tumors in an immune-competent, self-antigen setting. Clin Cancer Res. 2017; 23(10):2478-2490.
77. Z. Hao et al., T cell-specific ablation of Fas leads to Fas ligand-mediated lymphocyte depletion and inflammatory pulmonary fibrosis. J Exp Med. 2004; 199(10):1355-1365.
78. E. Jacoby et al., Murine allogeneic CD19 CAR T cells harbor potent antileukemic activity but have the potential to mediate lethal GVHD. Blood. 2016; 127(10):1361-1370.
79. Z. Zheng et al., Protein L: a novel reagent for the detection of chimeric antigen receptor (CAR) expression by flow cytometry. J Transl Med. 2012; 10-29.
80. G. Li et al., 4-1BB enhancement of CAR T function requires NF-κB and TRAFs. JCI Insight. 2018; 3(18):121322.

### Embodiments of the Presently Disclosed Subject Matter

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A dominant negative Fas polypeptide comprising a first modification in the cytoplasmic death domain and a second modification in the N-terminal region of human Fas, wherein the second modification is located between the peptide signal region and the cysteine rich domain 1 of human Fas.

2.-3. (canceled)

4. The dominant negative Fas polypeptide of claim 1, wherein
a) the first modification comprises or consists of a deletion of amino acids 230-314 of human Fas; or
b) the first modification comprises or consists of a point mutation at position 260 of human Fas.

5. (canceled)

6. The dominant negative Fas polypeptide of claim 4, wherein the point mutation is D260V.

7. (canceled)

8. The dominant negative Fas polypeptide of claim 7, wherein
the peptide signal region is encoded by amino acids 1 to 25 of human Fas and wherein the cysteine rich domain 1 of is encoded by amino acids 48 to 82 of human Fas.

9. (canceled)

10. The dominant negative Fas polypeptide of claim 8, wherein
a) the second modification comprises or consists of a modification at position 32 of human Fas;
b) the second modification comprises or consists of a deletion of amino acid 32 of human Fas;
c) the second modification comprises or consists of a deletion of amino acids 31 and 32 of human Fas;
d) the second modification comprises or consists of a modification at position 33 of human Fas;
e) the second modification comprises or consists of a deletion of amino acids 33 and 34 of human Fas; or
f) the second modification comprises or consists of a point mutation S32A of human Fas.

11.-13. (canceled)

14. The dominant negative Fas polypeptide of claim 1, wherein the dominant negative Fas polypeptide comprises or consists of an amino acid sequence that is at least about 80%, at least about 85%, at least about 90%, at least about 95%, at least about 96%, at least about 97%, at least about 98%, at least about 99%, or at least about 100% identical to the amino acid sequence set forth in SEQ ID NO: 16, SEQ ID NO: 18, SEQ ID NO: 20, SEQ ID NO: 22, or SEQ ID NO: 24.

15. The dominant negative Fas polypeptide of claim 1, wherein the dominant negative Fas polypeptide comprises or consists of the amino acid sequence set forth in SEQ ID NO: 16, SEQ ID NO: 18, SEQ ID NO: 20, SEQ ID NO: 22, SEQ ID NO: 24, or SEQ ID NO: 10.

16.-34. (canceled)

35. The dominant negative Fas polypeptide of claim 1, wherein the first modification prevents the binding between the dominant negative Fas polypeptide and a FADD polypeptide.

36. The dominant negative Fas polypeptide of claim 1, wherein the second modification increases (a) the surface expression of the dominant negative Fas polypeptide by a cell, and/or (b) the transduction efficiency of the dominant negative Fas polypeptide into a cell, and/or (c) the protection of the dominant negative Fas polypeptide from FasL-induced apoptosis.

37. A cell comprising a) an antigen-recognizing receptor that binds to an antigen, and b) a dominant negative Fas polypeptide of claim 1.

38. The cell of claim 37, wherein the dominant negative Fas polypeptide enhances cell persistence and/or reduces apoptosis or anergy of the cell.

39. (canceled)

40. The cell of claim 37, wherein the antigen-recognizing receptor is exogenous or endogenous.

41. The cell of claim 37, wherein the antigen-recognizing receptor and/or the dominant negative Fas polypeptide is expressed from a vector.

42.-43. (canceled)

44. The cell of claim 37, wherein the cell is an immunoresponsive cell selected from the group consisting of a cell of the lymphoid lineage, a cell of the myeloid lineage, a T cell, a Natural Killer (NK) cell, a B cell, a monocyte, and a macrophage.

45.-47. (canceled)

48. The cell of claim 44, wherein the T cell is a cytotoxic T lymphocyte (CTL), a regulatory T cell (Treg), or a Natural Killer T (NKT) cell.

49. The cell of claim 37, wherein the cell is autologous or allogeneic to the intended recipient.

50. The cell of claim 37, wherein the antigen is a tumor antigen or a pathogen antigen.

51.-52. (canceled)

53. The cell of claim 37, wherein
a) the tumor antigen is a tumor-specific antigen (TSA) or a tumor-associated antigen (TAA);
b) the tumor antigen is selected from the group consisting of CD19, MUC16, MUC1, CAIX, CEA, CD8, CD7, CD10, CD20, CD22, CD30, CLL1, CD33, CD34, CD38, CD41, CD44, CD49f, CD56, CD74, CD133, CD138, EGP-2, EGP-40, EpCAM, Erb-B2, Erb-B3, Erb-B4, FBP, Fetal acetylcholine receptor, folate receptor-α, GD2, GD3, HER-2, hTERT, IL-13R-α2, κ-light chain, KDR, mutant KRAS, mutant HRAS, mutant PIK3CA, mutant IDH, mutant p53, mutant NRAS, LeY, L1 cell adhesion molecule, MAGE-A1, Mesothelin, MAGEA3, CT83, p53, MART1, GP100, Proteinase3 (PR1), Tyrosinase, Survivin, hTERT, EphA2, NKG2D ligands, NY-ESO-1, oncofetal antigen (h5T4), PSCA, PSMA, ROR1, TAG-72, VEGF-R2, WT-1, BCMA, CD123, CD44V6, NKCS1, EGF1R, EGFR-VIII, CD99, CD70, ADGRE2, CCR1, LILRB2, PRAME, HPV E6 oncoprotein, HPV E7 oncoprotein, and ERBB, optionally wherein the antigen is CD19; or
c) the pathogen-associated antigen is a viral antigen present in Cytomegalovirus (CMV), a viral antigen present in Epstein Barr Virus (EBV), a viral antigen present in Human Immunodeficiency Virus (HIV), or a viral antigen present in influenza virus.

54.-57. (canceled)

58. The cell of claim 37, wherein
a) the antigen-recognizing receptor is a TCR that recognizes a pathogen-associated antigen, and the cell is a pathogen-specific T cell;
b) the antigen-recognizing receptor is a TCR that recognizes a tumor antigen, and the cell is a tumor-specific T cell.

59.-60. (canceled)

61. The cell of claim 37, wherein the antigen-recognizing receptor is a CAR comprising an extracellular antigen-binding domain, a transmembrane domain, and an intracellular signaling domain.

62. (canceled)

63. The cell of claim 61, wherein the intracellular signaling domain comprises a native or a modified CD3ζ polypeptide.

64. (canceled)

65. The cell of claim 63, wherein the modified CD3ζ polypeptide comprises a native ITAM1, an ITAM2 variant consisting of two loss-of-function mutations, and an ITAM3 consisting of two loss-of-function mutations.

66. The cell of claim 61, wherein the intracellular signaling domain further comprises at least one co-stimulatory signaling region.

67. The cell of claim 66, wherein the at least one co-stimulatory signaling region comprises a CD28 polypeptide, a 4-1BB polypeptide, an OX40 polypeptide, an ICOS polypeptide, a DAP-10 polypeptide, or a combination thereof, optionally wherein the at least one co-stimulatory signaling region comprises a CD28 polypeptide.

68. (canceled)

69. The cell of claim 37, further comprising a suicide gene, optionally wherein the suicide gene is a Herpes simplex virus thymidine kinase (hsv-tk), inducible Caspase 9 Suicide gene (iCasp-9) or a truncated human epidermal growth factor receptor (EGFRt) polypeptide.

70. (canceled)

71. A nucleic acid composition comprising (a) a first nucleic acid sequence encoding an antigen-recognizing receptor that binds to an antigen, and (b) a second nucleic acid sequence encoding a dominant negative Fas polypeptide of claim 1.

72. The nucleic acid composition of claim 71, wherein one or both of the first and second nucleic acid sequences are operably linked to a promoter element or are present on a vector, optionally wherein the vector is a retroviral or a lentiviral vector.

73.-76. (canceled)

77. A vector comprising the nucleic acid composition of claim 72.

78. (canceled)

79. A pharmaceutical composition comprising an effective amount of cells of claim 37, and a pharmaceutically acceptable excipient.

80. (canceled)

81. A method of inducing and/or enhancing an immune response to a target antigen, reducing tumor burden in a subject, treating and/or preventing a neoplasm, and/or lengthening survival of a subject having a neoplasm, the method comprising administering to the subject an effective amount of the cells of claim 37.

82. (canceled)

83. The method of claim 81, wherein the method reduces the number of tumor cells, reduces tumor size, and/or eradicates the tumor in the subject.

84.-86. (canceled)

87. The method of claim 81, wherein
a) the tumor or neoplasm is selected from the group consisting of B cell leukemia, multiple myeloma, acute lymphoblastic leukemia (ALL), chronic lymphocytic leukemia (CLL), non-Hodgkin's lymphoma, myeloid leukemias, and myelodysplastic syndrome (MDS); or
b) the tumor or neoplasm is a solid tumor originating from the brain, breast, lung, gastro-intestinal tract (including esophagus, stomach, small intestine, large intestine, and rectum), pancreas, prostate, soft tissue/bone, uterus, cervix, ovary, kidney, skin, thymus, testis, head and neck, or liver.

88.-89. (canceled)

90. A method of preventing and/or treating a pathogen infection in a subject, the method comprising administering to the subject an effective amount of the cells of claim 37.

91. (canceled)

92. A method for producing an antigen-specific cell, the method comprising introducing into a cell (a) a first nucleic acid sequence encoding an antigen-recognizing receptor that binds to an antigen; and (b) a second nucleic sequence encoding an dominant negative Fas polypeptide of claim 1.

93.-95. (canceled)

96. A kit comprising a cell of claim 37, wherein the kit further comprises written instructions for treating and/or preventing a neoplasm or a pathogen infection.

97. (canceled)


SEQUENCE LISTING (74 sequences)

SEQ ID NO: 1 - protein, 15 aa, synthetic construct
  REGION 1..15: note = Description of Artificial Sequence: Syntheticpeptide
GGGGSGGGGSGGGGS

SEQ ID NO: 2 - protein, 20 aa, Homo sapiens
MYRMQLLSCIALSLALVTNS

SEQ ID NO: 3 - protein, 20 aa, Mus musculus
MYSMQLASCVTLTLVLLVNS

SEQ ID NO: 4 - protein, 20 aa, Homo sapiens
METPAQLLFLLLLWLPDTTG

SEQ ID NO: 5 - protein, 20 aa, Mus musculus
METDTLLLWVLLLWVPGSTG

SEQ ID NO: 6 - protein, 21 aa, Homo sapiens
MALPVTALLLPLALLLHAARP

SEQ ID NO: 7 - protein, 18 aa, Homo sapiens
MALPVTALLLPLALLLHA

SEQ ID NO: 8 - protein, 16 aa, Homo sapiens
MKWVTFISLLFSSAYS

SEQ ID NO: 9 - protein, 30 aa, Homo sapiens
MDSKGSSQKGSRLLLLLVVSNLLLCQGVVS

SEQ ID NO: 10 - protein, 335 aa, Homo sapiens
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 11 - DNA, 1005 bp, Homo sapiens
  source 1..1005: mol_type = other DNA
atgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccagatctaacttggggtggctttgtcttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtc

SEQ ID NO: 12 - protein, 250 aa, synthetic construct
  REGION 1..250: note = Description of Artificial Sequence: Syntheticpolypeptide
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLLKDITSDSENSNFRNEIQSLV

SEQ ID NO: 13 - DNA, 750 bp, synthetic construct
  misc_feature 1..750: note = Description of Artificial Sequence: Syntheticpolynucleotide
  source 1..750: mol_type = other DNA
atgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggttccagatctaacttggggtggctttgtcttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgctcaaggacattactagtgactcagaaaattcaaacttcagaaatgaaatccaaagcttggtc

SEQ ID NO: 14 - protein, 335 aa, synthetic construct
  REGION 1..335: note = Description of Artificial Sequence: Syntheticpolypeptide
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLSKYITTIAGVMTLSQVKGFVRKNGVNEAKIVEIKNDNVQDTAEQKVQLLRNWHQLHGKKEAYDTLIKDLKKANLCTLAEKIQTIILKDITSDSENSNFRNEIQSLV

SEQ ID NO: 15 - DNA, 1005 bp, synthetic construct
  misc_feature 1..1005: note = Description of Artificial Sequence: Syntheticpolynucleotide
  source 1..1005: mol_type = other DNA
atgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccagatctaacttggggtggctttgtcttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagttgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtc

SEQ ID NO: 16 - protein, 249 aa, synthetic construct
  REGION 1..249: note = Description of Artificial Sequence: Syntheticpolypeptide
MLGIWTLLPLVLTSVARLSSKSVNAQVTDINKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLLKDITSDSENSNFRNEIQSLV

SEQ ID NO: 17 - DNA, 747 bp, synthetic construct
  misc_feature 1..747: note = Description of Artificial Sequence: Syntheticpolynucleotide
  source 1..747: mol_type = other DNA
atgctgggcatctggaccctcctacctctggttcttacgtctgttgctagattatcgtccaaaagtgttaatgcccaagtgactgacatcaacaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggttccagatctaacttggggtggctttgtcttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgctcaaggacattactagtgactcagaaaattcaaacttcagaaatgaaatccaaagcttggtc

SEQ ID NO: 18 - protein, 248 aa, synthetic construct
  REGION 1..248: note = Description of Artificial Sequence: Syntheticpolypeptide
MLGIWTLLPLVLTSVARLSSKSVNAQVTDIKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLLKDITSDSENSNFRNEIQSLV

SEQ ID NO: 19 - DNA, 744 bp, synthetic construct
  misc_feature 1..744: note = Description of Artificial Sequence: Syntheticpolynucleotide
  source 1..744: mol_type = other DNA
atgctgggcatctggaccctcctacctctggttcttacgtctgttgctagattatcgtccaaaagtgttaatgcccaagtgactgacatcaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggttccagatctaacttggggtggctttgtcttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgctcaaggacattactagtgactcagaaaattcaaacttcagaaatgaaatccaaagcttggtc

SEQ ID NO: 20 - protein, 248 aa, synthetic construct
  REGION 1..248: note = Description of Artificial Sequence: Syntheticpolypeptide
MLGIWTLLPLVLTSVARLSSKSVNAQVTDINGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLLKDITSDSENSNFRNEIQSLV

SEQ ID NO: 21 - DNA, 744 bp, synthetic construct
  misc_feature 1..744: note = Description of Artificial Sequence: Syntheticpolynucleotide
  source 1..744: mol_type = other DNA
atgctgggcatctggaccctcctacctctggttcttacgtctgttgctagattatcgtccaaaagtgttaatgcccaagtgactgacatcaacggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggttccagatctaacttggggtggctttgtcttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgctcaaggacattactagtgactcagaaaattcaaacttcagaaatgaaatccaaagcttggtc

SEQ ID NO: 22 - protein, 248 aa, synthetic construct
  REGION 1..248: note = Description of Artificial Sequence: Syntheticpolypeptide
MLGIWTLLPLVLTSVARLSSKSVNAQVTDINSLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLLKDITSDSENSNFRNEIQSLV

SEQ ID NO: 23 - DNA, 744 bp, synthetic construct
  misc_feature 1..744: note = Description of Artificial Sequence: Syntheticpolynucleotide
  source 1..744: mol_type = other DNA
atgctgggcatctggaccctcctacctctggttcttacgtctgttgctagattatcgtccaaaagtgttaatgcccaagtgactgacatcaactcattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggttccagatctaacttggggtggctttgtcttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgctcaaggacattactagtgactcagaaaattcaaacttcagaaatgaaatccaaagcttggtc

SEQ ID NO: 24 - protein, 250 aa, synthetic construct
  REGION 1..250: note = Description of Artificial Sequence: Syntheticpolypeptide
MLGIWTLLPLVLTSVARLSSKSVNAQVTDINAKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLLKDITSDSENSNFRNEIQSLV

SEQ ID NO: 25 - DNA, 750 bp, synthetic construct
  misc_feature 1..750: note = Description of Artificial Sequence: Syntheticpolynucleotide
  source 1..750: mol_type = other DNA
atgctgggcatctggaccctcctacctctggttcttacgtctgttgctagattatcgtccaaaagtgttaatgcccaagtgactgacatcaacgccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggttccagatctaacttggggtggctttgtcttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgctcaaggacattactagtgactcagaaaattcaaacttcagaaatgaaatccaaagcttggtc

SEQ ID NO: 26 - protein, 272 aa, Homo sapiens
PEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWK

SEQ ID NO: 27 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 28 - protein, 247 aa, Mus musculus
MASPLTRFLSLNLLLMGESIILGSGEAKPQAPELRIFPKKMDAELGQKVDLVCEVLGSVSQGCSWLFQNSSSKLPQPTFVVYMASSHNKITWDEKLNSSKLFSAVRDTNNKYVLTLNKFSKENEGYYFCSVISNSVMYFSSVVPVLQKVNSTTTKPVLRTPSPVHPTGTSQPQRPEDCRPRGSVKGTGLDFACDIYIWAPLAGICVAPLLSLIITLICYHRSRKRVCKCPRPLVRQEGKPRPSEKIV

SEQ ID NO: 29 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 30 - DNA, 81 bp, Homo sapiens
  source 1..81: mol_type = other DNA
ttttgggtgctggtggtggttggtggagtcctggcttgctatagcttgctagtaacagtggcctttattattttctgggtg

SEQ ID NO: 31 - protein, 218 aa, Mus musculus
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 32 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 33 - protein, 188 aa, Mus musculus
MKWKVSVLACILHVRFPGAEAQSFGLLDPKLCYLLDGILFIYGVIITALYLRAKFSRSAETAANLQDPNQLYNELNLGRREEYDVLEKKRARDPEMGGKQRRRNPQEGVYNALQKDKMAEAYSEIGTKGERRRGKGHDGLYQDSHFQAVQFGNRREREGSELTRTLGLRARPKACRHKKPLSLPAAVS

SEQ ID NO: 34 - protein, 112 aa, Homo sapiens
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - protein, 112 aa, synthetic construct
  REGION 1..112: note = Description of Artificial Sequence: Syntheticpolypeptide
RVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 36 - DNA, 336 bp, synthetic construct
  misc_feature 1..336: note = Description of Artificial Sequence: Syntheticpolynucleotide
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccaggggctcagtacagccaccaaggacaccttcgacgcccttcacatgcaggccctgccccctcgc

SEQ ID NO: 37 - protein, 22 aa, synthetic construct
  REGION 1..22: note = Description of Artificial Sequence: Syntheticpeptide
QNQLYNELNLGRREEYDVLDKR

SEQ ID NO: 38 - DNA, 66 bp, synthetic construct
  misc_feature 1..66: note = Description of Artificial Sequence: Syntheticoligonucleotide
  source 1..66: mol_type = other DNA
cagaaccagctctataacgagctcaatctaggacgaagagaggagtacgatgttttggacaagaga

SEQ ID NO: 39 - protein, 22 aa, synthetic construct
  REGION 1..22: note = Description of Artificial Sequence: Syntheticpeptide
QNQLFNELNLGRREEFDVLDKR

SEQ ID NO: 40 - DNA, 66 bp, synthetic construct
  misc_feature 1..66: note = Description of Artificial Sequence: Syntheticoligonucleotide
  source 1..66: mol_type = other DNA
cagaaccagctctttaacgagctcaatctaggacgaagagaggagttcgatgttttggacaagaga

SEQ ID NO: 41 - protein, 23 aa, synthetic construct
  REGION 1..23: note = Description of Artificial Sequence: Syntheticpeptide
QEGLYNELQKDKMAEAYSEIGMK

SEQ ID NO: 42 - DNA, 69 bp, synthetic construct
  misc_feature 1..69: note = Description of Artificial Sequence: Syntheticoligonucleotide
  source 1..69: mol_type = other DNA
caggaaggcctgtacaatgaactgcagaaagataagatggcggaggcctacagtgagattgggatgaaa

SEQ ID NO: 43 - protein, 23 aa, synthetic construct
  REGION 1..23: note = Description of Artificial Sequence: Syntheticpeptide
QEGLFNELQKDKMAEAFSEIGMK

SEQ ID NO: 44 - DNA, 69 bp, synthetic construct
  misc_feature 1..69: note = Description of Artificial Sequence: Syntheticoligonucleotide
  source 1..69: mol_type = other DNA
caggaaggcctgttcaatgaactgcagaaagataagatggcggaggccttcagtgagattgggatgaaa

SEQ ID NO: 45 - protein, 22 aa, synthetic construct
  REGION 1..22: note = Description of Artificial Sequence: Syntheticpeptide
HDGLYQGLSTATKDTYDALHMQ

SEQ ID NO: 46 - DNA, 66 bp, synthetic construct
  misc_feature 1..66: note = Description of Artificial Sequence: Syntheticoligonucleotide
  source 1..66: mol_type = other DNA
cacgatggcctttaccagggtctcagtacagccaccaaggacacctacgacgcccttcacatgcag

SEQ ID NO: 47 - protein, 22 aa, synthetic construct
  REGION 1..22: note = Description of Artificial Sequence: Syntheticpeptide
HDGLFQGLSTATKDTFDALHMQ

SEQ ID NO: 48 - DNA, 66 bp, synthetic construct
  misc_feature 1..66: note = Description of Artificial Sequence: Syntheticoligonucleotide
  source 1..66: mol_type = other DNA
cacgatggccttttccaggggctcagtacagccaccaaggacaccttcgacgcccttcacatgcag

SEQ ID NO: 49 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 50 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 51 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 52 - protein, 484 aa, synthetic construct
  REGION 1..484: note = Description of Artificial Sequence: Syntheticpolypeptide
ALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - DNA, 1452 bp, synthetic construct
  misc_feature 1..1452: note = Description of Artificial Sequence: Syntheticpolynucleotide
  source 1..1452: mol_type = other DNA
gctctcccagtgactgccctactgcttcccctagcgcttctcctgcatgcagaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacgggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 54 - protein, 485 aa, synthetic construct
  REGION 1..485: note = Description of Artificial Sequence: Syntheticpolypeptide
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 55 - DNA, 1458 bp, synthetic construct
  misc_feature 1..1458: note = Description of Artificial Sequence: Syntheticpolynucleotide
  source 1..1458: mol_type = other DNA
atggctctcccagtgactgccctactgcttcccctagcgcttctcctgcatgcagaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacgggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccaggggctcagtacagccaccaaggacaccttcgacgcccttcacatgcaggccctgccccctcgctaa

SEQ ID NO: 56 - protein, 334 aa, synthetic construct
  REGION 1..334: note = Description of Artificial Sequence: Syntheticpolypeptide
MLGIWTLLPLVLTSVARLSSKSVNAQVTDINKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 57 - DNA, 1002 bp, synthetic construct
  misc_feature 1..1002: note = Description of Artificial Sequence: Syntheticpolynucleotide
  source 1..1002: mol_type = other DNA
atgctgggcatctggaccctcctacctctggttcttacgtctgttgctagattatcgtccaaaagtgttaatgcccaagtgactgacatcaacaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccagatctaacttggggtggctttgtcttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtc

SEQ ID NO: 58 - protein, 333 aa, synthetic construct
  REGION 1..333: note = Description of Artificial Sequence: Syntheticpolypeptide
MLGIWTLLPLVLTSVARLSSKSVNAQVTDIKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 59 - DNA, 999 bp, synthetic construct
  misc_feature 1..999: note = Description of Artificial Sequence: Syntheticpolynucleotide
  source 1..999: mol_type = other DNA
atgctgggcatctggaccctcctacctctggttcttacgtctgttgctagattatcgtccaaaagtgttaatgcccaagtgactgacatcaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccagatctaacttggggtggctttgtcttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtc

SEQ ID NO: 60 - protein, 333 aa, synthetic construct
  REGION 1..333: note = Description of Artificial Sequence: Syntheticpolypeptide
MLGIWTLLPLVLTSVARLSSKSVNAQVTDINGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 61 - DNA, 999 bp, synthetic construct
  misc_feature 1..999: note = Description of Artificial Sequence: Syntheticpolynucleotide
  source 1..999: mol_type = other DNA
atgctgggcatctggaccctcctacctctggttcttacgtctgttgctagattatcgtccaaaagtgttaatgcccaagtgactgacatcaacggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccagatctaacttggggtggctttgtcttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtc

SEQ ID NO: 62 - protein, 335 aa, synthetic construct
  REGION 1..335: note = Description of Artificial Sequence: Syntheticpolypeptide
MLGIWTLLPLVLTSVARLSSKSVNAQVTDINAKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 63 - DNA, 1005 bp, synthetic construct
  misc_feature 1..1005: note = Description of Artificial Sequence: Syntheticpolynucleotide
  source 1..1005: mol_type = other DNA
atgctgggcatctggaccctcctacctctggttcttacgtctgttgctagattatcgtccaaaagtgttaatgcccaagtgactgacatcaacgccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccagatctaacttggggtggctttgtcttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtc

SEQ ID NO: 64 - protein, 333 aa, synthetic construct
  REGION 1..333: note = Description of Artificial Sequence: Syntheticpolypeptide
MLGIWTLLPLVLTSVARLSSKSVNAQVTDINSLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 65 - DNA, 999 bp, synthetic construct
  misc_feature 1..999: note = Description of Artificial Sequence: Syntheticpolynucleotide
  source 1..999: mol_type = other DNA
atgctgggcatctggaccctcctacctctggttcttacgtctgttgctagattatcgtccaaaagtgttaatgcccaagtgactgacatcaactcattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccagatctaacttggggtggctttgtcttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtc

SEQ ID NO: 66 - RNA, 20 nt, synthetic construct
  misc_feature 1..20: note = Description of Artificial Sequence: Syntheticoligonucleotide
  source 1..20: mol_type = other RNA
gtgactgacatcaactccaa

SEQ ID NO: 67 - DNA, 23 bp, synthetic construct
  misc_feature 1..23: note = Description of Artificial Sequence: Syntheticprimer
  source 1..23: mol_type = other DNA
tctatcattcatggtgctgtttc

SEQ ID NO: 68 - DNA, 22 bp, synthetic construct
  misc_feature 1..22: note = Description of Artificial Sequence: Syntheticprimer
  source 1..22: mol_type = other DNA
aggggaaccaaaaactgtaaaa

SEQ ID NO: 69 - DNA, 33 bp, Homo sapiens
  source 1..33: mol_type = other DNA
caagtgactgacatcaactccaagggattggaa

SEQ ID NO: 70 - protein, 11 aa, Homo sapiens
QVTDINSKGLE

SEQ ID NO: 71 - DNA, 33 bp, synthetic construct; identical to SEQ ID NO: 69
  misc_feature 1..33: note = Description of Artificial Sequence: Syntheticoligonucleotide
  misc_feature 18..23: note = This region may or may not be present
  source 1..33: mol_type = other DNA

SEQ ID NO: 72 - protein, 11 aa, synthetic construct
  REGION 1..11: note = Description of Artificial Sequence: Syntheticpeptide
  REGION 6..7: note = MISC_FEATURE - This region may or may not be present
QVTDINSKGLE

SEQ ID NO: 73 - DNA, 33 bp, synthetic construct; identical to SEQ ID NO: 69
  misc_feature 1..33: note = Description of Artificial Sequence: Syntheticoligonucleotide
  misc_feature 7..25: note = This region may or may not be present
  source 1..33: mol_type = other DNA

SEQ ID NO: 74 - protein, 5 aa, synthetic construct
  REGION 1..5: note = Description of Artificial Sequence: Syntheticpeptide
QVDWN